SEQUENCE LISTING (41 sequences)

SEQ ID NO: 1 - DNA, 6196 bp, Homo Sapiens
tgaggaactgcgtgtggagtcagcccagtctggatgcacaggaggatgctggcggcacagtgagtgaggcctggtgccagagctgtgcggaccccttgttggccatggagcagcaggcccagaggccctctccccagccctgcttgcctgcctcggagaggacagaggcctaggcccacgggggagggtgttggcagacagatgccctccaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcacgggcagcagcgggacccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccagtggcacccctagctgttcctggaaggacctgaagaccatgaccaatttctacctggtcctgttctacatcaccctgcttacactctccagacagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgagctcctactgaagcccaagttcagcggcgtggagaagatcaagaccatcggcagcacgtacatggcagctgcagggctcagcgtcgcctcagggcacgagaaccaggagctggagcggcagcatgcccacattggtgtcatggtggagttcagcatcgccctgatgagtaagctggacggcatcaacaggcactccttcaactccttccgcctccgcgtcggcataaaccatgggcctgtgattgctggagtgattggggcccgaaaacctcagtatgacatctggggaaacactgtcaatgtggccagccgaatggaaagcactggagaacttgggaaaatccaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactgagggctcctgctggattccgaaaaggccgggaagccagtctccttccctgaagcaagcccaggagaagactctccgccccacgccaatcccaaaggcatgcagatggctgtgcatgttggcttctttggacctgcactggaggatttctcagacacatgcaccagattctggctcgaagcagccactgagccataatgcgcaggggaggccagaagctctgtgcctggtctgtaacagtttccaggccagctggagaatgttcactggttcggggctgactttgagatctttgttccctgaggtgccaggcaggcaactttagcacatgatgaaaacagacttccacctcagtggcctgtgggcacgcacaagtgaggtctgtttttctagacaccaagggggagtaagctgagctgtctagcacggattggagactccctctccctggtgggcctggcaatgacagcatttctcacagaggcattctggtaaatgaagctgaaaggggtgttttacatctgtaaacggtttcaaacaggtagagagaaaaacaccacaattaacactgttactttttgccttgtctggcatgtttgttttaaatgaatacattaatggggtttttatccttttgaatgacttttcagacactagacataaatctcttccctccagtgtatgctctgcctttttaaccactgacatgtaaggaggactactgtctagcatcagcttatggggtcagctggctgtggggatagagtcctgaggaatgtggtcacagcaagaaggcggggagcagcagagccttgcctttgaatgaggcagcttgtgaggcaagcattctggagagaggtgctttgaaagtaaggtgcggcctttcacctcttccttgattactcacacatctttgcgttctcccctgccgtccttcaactgtatcttacttttcttaccagaaaggaatggagtctgtttagagacaacttggacaacctgtgagtgcatctcttctttcctttagtcttcacagctaactctggagagcttcaaaactagaaggatctactccgcatgggtgcatgcagaggctcctggatctgggaagcccgccccctcacaaatgctgagccgttcttgctctgaaactgcgtgagtcaaggcaaatgcaaaaagccaggttttggggatgtgtcttactgtgcttcaacttcccaaggaattgaaagtcaacctaactgtaacaacagggtgagaaatgaccaaactgcccgtgactttttctgaatggacttcataaccggaagacttaaccggtggcctcatcaccagagcatcgccaggatttctaatgcactcagtttccctacatagcagggattcttagctaggtgtccccatgaaccccgtaaagttctacacaaagtcttgcatacaggagcctttacaagatgattatacagggttgcagattgggtgactgaccagacttgttggggtcctgggatgagttgccccgggctgcaaattaagagtacagctaagtgcgggggtggcggtggagggaacgaaaattgaacctgtctgcctgtgctgtgtcgtgtggctttatcagcccgaggaagggcaggtgtattctaatttgcacaaaggtgctgggtagactagtggcagctctcatgtgctgcacataagtggaatcagtatgaatagaagaacttgctgtataaaggaatttcatggcaacaatgctggtaagggcaattagcctcgcttaagttgccttttttacacaccaaaactttttacatgaagggctggtttcacatgaatactatactgaaatctgtgccacaccaaaactttttacatgaagggctggtttcacatgaatactatactgaaatctgtgctctcaagatctagcagtgaccagggctgcccggcgggggctctcctggcaagtcaggaaggtttctgttgctaatataacatagaaacacattagtgcactgggcctctctgaggtcagcatatttgtactcttggaatatttgtttttttcttcagtaacaacagaaaccccagttgggagtttaacaaataactgactaccactcactcatgcatttttatttccaattaaagcaaagcactgtgctgtgctcagataataatagtttgtaagtaaaagtttttagttttcagtgttcaggttatagaatataactgaccataaaaattacctgcaggtattttctttttatgaacttgtttttaaattaccaagtaattactggtgtcattttgttttatgacagacacacgtatctaacaaacaaacaaacagtgaccttctccatgggtcaaggacttccttacaatttctcctgagttaacttttgtgaaaataatacctaaggttttctggcttattgaggaaatttcctaacaaacaaacaaacaaacaaacagaagagaagatcattaaccactgtatactttgtgtatataataggtcagtgtaaagaaatatgatttgaggtggtgcatgcaagtaactagggtttattctatataatgaatatttatagatctgtaacatttgtttcaaaatgctgtttcatttttataaagtaccagtgtttagctgctttttatacattaaattagcaatttgaaaaactc

SEQ ID NO: 2 - protein, 1080 aa, Homo Sapiens
MPAKGRYFLNEGEEGPDQDALYEKYQLTSQHGPLLLTLLLVAATACVALIIIAFSQGDPSRHQAILGMAFLVLAVFAALSVLMYVECLLRRWLRALALLTWACLVALGYVLVFDAWTKAACAWEQVPFFLFIVFVVYTLLPFSMRGAVAVGAVSTASHLLVLGSLMGGFTTPSVRVGLQLLANAVIFLCGNLTGAFHKHQMQDASRDLFTYTVKCIQIRRKLRIEKRQQENLLLSVLPAHISMGMKLAIIERLKEHGDRRCMPDNNFHSLYVKRHQNVSILYADIVGFTQLASDCSPKELVVVLNELFGKFDQIAKANECMRIKILGDCYYCVSGLPVSLPTHARNCVKMGLDMCQAIKQVREATGVDINMRVGIHSGNVLCGVIGLRKWQYDVWSHDVSLANRMEAAGVPGRVHITEATLKHLDKAYEVEDGHGQQRDPYLKEMNIRTYLVIDPRSQQPPPPSQHLPRPKGDAALKMRASVRMTRYLESWGAARPFAHLNHRESVSSGETHVPNGRRPKSVPQRHRRTPDRSMSPKGRSEDDSYDDEMLSAIEGLSSTRPCCSKSDDFYTFGSIFLEKGFEREYRLAPIPRARHDFACASLIFVCILLVHVLLMPRTAALGVSFGLVACVLGLVLGLCFATKFSRCCPARGTLCTISERVETQPLLRLTLAVLTIGSLLTVAIINLPLMPFQVPELPVGNETGLLAASSKTRALCEPLPYYTCSCVLGFIACSVFLRMSLEPKVVLLTVALVAYLVLFNLSPCWQWDCCGQGLGNLTKPNGTTSGTPSCSWKDLKTMTNFYLVLFYITLLTLSRQIDYYCRLDCLWKKKFKKEHEEFETMENVNRLLLENVLPAHVAAHFIGDKLNEDWYHQSYDCVCVMFASVPDFKVFYTECDVNKEGLECLRLLNEIIADFDELLLKPKFSGVEKIKTIGSTYMAAAGLSVASGHENQELERQHAHIGVMVEFSIALMSKLDGINRHSFNSFRLRVGINHGPVIAGVIGARKPQYDIWGNTVNVASRMESTGELGKIQVTEETCTILQGLGYSCECRGLINVKGKGELRTYFVCTDTAKFQGLGLN

SEQ ID NO: 3 - DNA, 1506 bp, Homo Sapiens
aggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccatcaggcagtggctgttggaaagatttaagatttcacacctatgacatacatgggatagcacctgggccgccatgcactcaataaagaatgtatttt

SEQ ID NO: 4 - protein, 244 aa, Homo Sapiens
MKKLMVVLSLIAAAWAEEQNKLVHGGPCDKTSHPYQAALYTSGHLLCGGVLIHPLWVLTAAHCKKPNLQVFLGKHNLRQRESSQEQSSVVRAVIHPDYDAASHDQDIMLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAK

SEQ ID NO: 5 - DNA, 1506 bp, Homo Sapiens; identical to SEQ ID NO: 3
  CDS (246)...(980)

SEQ ID NO: 6 - DNA, 4282 bp, Homo Sapiens
ctctctctctttcgggcggagtcgcccaccactgccagcccagcgctggggggacctgctccaggctgtagccgcaggaccccaccaccccccatggctcccctggccttggtgggggtcacactcctcctggcggctcccccatgctccggggcagccaccccaaccccctccctgccgcctcccccggccaatgacagcgacaccagcacagggggctgccaggggtcctaccgctgccagccgggggtgctgctgcccgtgtgggagcccgacgacccgtcgctgggtgacaaggcggcacgggcagtggtgtactttgtggccatggtctacatgtttctgggagtgtccatcatcgccgaccgtttcatggcggccatcgaggtcatcacgtcaaaagagaaggagatcaccatcaccaaggccaacggtgagaccagcgtgggcaccgttcgcatctggaatgagacggtgtccaacctcacgctcatggccctgggctcctccgcacctgagatcctgctgtcagtcatcgaagtctgcggccacaacttccaggcgggtgagctgggcccaggcaccatcgtgggcagcgctgccttcaacatgtttgtggtcatcgccgtgtgcatctacgtcatcccagccggcgagagccgcaagatcaagcacctgagagtcttctttgtcactgcctcttggagcatcttcgcctatgtctggctttatctcatccttgctgttttttcccccggtgtggtccaggtgtgggaggcgctgctgaccctggtcttcttcccggtgtgcgtggtattcgcctggatggccgacaagcggctgctcttctacaagtacgtgtacaagcgctaccgcaccgacccacgcagcggcatcatcataggcgccgagggcgaccccccgaagagcatcgagctggacggcacgttcgtgggcgccgaggccccaggtgagctgggcggcctgggcccgggccccgccgaggcgcgcgagctggacgccagccgccgcgaggtcatccagatcctcaaggacctcaagcagaagcacccggacaaggatctggagcagctggtgggcatcgccaactactacgcgctgctgcaccagcagaagagccgcgccttctaccgcatccaggccacgcggctgatgaccggcgccgggaacgtgctgcgcagacacgcggcggacgcctcgcgcagggcggcgccggccgagggcgcgggcgaggacgaagacgacggcgccagccgcatcttcttcgagcctagcctctaccactgcctggagaactgcggctccgtgctgctgtccgtcacgtgccagggcggcgagggcaacagcaccttctacgtggactaccgcactgaggacggctctgccaaggcgggctccgactacgagtacagcgagggcacgctggtgttcaaaccaggcgagacgcagaaggagctgcgcatcggcatcatcgacgacgacatcttcgaggaggacgagcatttcttcgtgcggctgctgaacctgcgcgtgggcgacgcgcagggcatgttcgagccggacggcggcgggcggcccaaggggcggctggtggcgccgctgctggccaccgtcaccatcctggacgacgaccacgcaggcatcttctccttccaggaccgcctgctgcacgtgagcgagtgcatgggcaccgtggacgtgcgcgtcgtgcgcagctcgggcgcgcgcggcaccgtgcgccttccctaccgcacggtggacggcacggcgcgcggcggcggcgtgcactacgaggacgcgtgcggagagctggagtttggcgacgacgagaccatgaaaactcttcaggtgaagatagttgatgacgaggaatatgagaaaaaggataatttcttcattgagctgggccagccccagtggcttaagcgagggatttcagctctgctactcaatcaaggggatggggacaggaagctaacagccgaggaggaggaggctcggaggatagcagagatgggcaagccagttcttggggagaactgccggctggaggtcatcatcgaggagtcatatgattttaagaacacggtggataaactcatcaagaaaacgaacttggccttggtaattgggacccattcatggagggagcagtttttagaggcaattacggtgagcgcaggggacgaggaggaggaggaggacgggtcccgggaggagcggctgccgtcgtgctttgactacgtgatgcacttcctgacggtgttctggaaggtgctcttcgcctgtgtgccccccaccgagtactgccacggctgggcctgctttggtgtctccatcctggtcatcggcctgctcaccgccctcattggggacctcgcctcccacttcggctgcaccgttggcctcaaggactctgtcaatgctgttgtcttcgttgccctgggcacctccatccctgacacgttcgccagcaaggtggcggcgctgcaggaccagtgcgccgacgcgtccatcggcaacgtgaccggctccaacgcggtgaacgtgttccttggcctgggcgtcgcctggtctgtggccgccgtgtactgggcggtgcagggccgccccttcgaggtgcgcactggcacgctggccttctccgtcacgctcttcaccgtcttcgccttcgtgggcattgccgtgctgctgtaccggcgccggccgcacatcggcggcgagctgggcggcccgcgcggacccaagctcgccaccaccgcgctcttcctgggcctctggctcctgtacatcctcttcgccagcctggaggcgtactgccacatccggggcttctagggcctcgcgcagagactcgtccccaccgcccgcccggggctagggactcggctgcacctgctcttggaccctggtctccttttccccccagactcggcctcctctcctgggactcggcctcccttctccgccccctcccctggctttgattgcccctgttctgtgtccccagtagctcagccttccctcttcctctcgggagcctccccggttcctcccctgcggtgaccccaactccagcccatcctgttggtgaccgtctatatccctggggaaatttccaccccagtcccctccccagggaaccacccccagtaaccatcctggggagtttaaggtctctctccttggtcacccagcctggctttgcccccaaagtctcccttcccctagtgacccccccccacttcaccccatgtcccagagcctcagaacccaccctccctgggggaccctcgaaggaggctgtcagaggccgtctcagctcccagcccttccccccagccctcagggagctccgctcagccccggcggggaggagcgggtgggtgtgcgcgcaaggaggccgcacacctttccttccaatccctccactcgggttcttgggaggacactcattctccaggctcggagacgaggggagaagtttggggtttcagtcccagggcttagccggaggaagcacattttgaacctgcaacttcagacattccagctcccccactcgccctccactacctctgagagcccagccacgccttggagggaggggcttgtgtgtgtatatagtgtgtttgggggaggggggacgcgggagggtgcatgtcttgggaaaagggggtgacagacaacttttgagagggcagcagactccctcagccatgagaaccagctttggggaggaggccgggaatcaaagcgagtccagttgatctcccctgacaatctggaaggttcattttgccctcagtgccagccaatccgggcaggaccctcgaagaggagaccgagggtcccagaggaccaatgctacaagccagcaaatgctgccacatctctgcctgatggggggtggggatgggtggggggatgggactgggccaagggatctgggtgggcatttttaactttggaggccttccatctgtcggtaggccatctgcattttcttactgttgatgtttcctgcccaaaggacacatttgggcagtgccacccactccttgggcccctaggatgacccaactacccccataactttctgcttcccacaggttttcagcattctatcgtcctgttgtgctcagccccaacatcccagacccgttacccgctacccttctctcccccagctcatcatcagtcgctgtctcttttctgtgatttctgtaaaagttgccataaaactttgaaattctgcctg

SEQ ID NO: 7 - protein, 921 aa, Homo Sapiens
MAPLALVGVTLLLAAPPCSGAATPTPSLPPPPANDSDTSTGGCQGSYRCQPGVLLPVWEPDDPSLGDKAARAVVYFVAMVYMFLGVSIIADRFMAAIEVITSKEKEITITKANGETSVGTVRIWNETVSNLTLMALGSSAPEILLSVIEVCGHNFQAGELGPGTIVGSAAFNMFVVIAVCIYVIPAGESRKIKHLRVFFVTASWSIFAYVWLYLILAVFSPGVVQVWEALLTLVFFPVCVVFAWMADKRLLFYKYVYKRYRTDPRSGIIIGAEGDPPKSIELDGTFVGAEAPGELGGLGPGPAEARELDASRREVIQILKDLKQKHPDKDLEQLVGIANYYALLHQQKSRAFYRIQATRLMTGAGNVLRRHAADASRRAAPAEGAGEDEDDGASRIFFEPSLYHCLENCGSVLLSVTCQGGEGNSTFYVDYRTEDGSAKAGSDYEYSEGTLVFKPGETQKELRIGIIDDDIFEEDEHFFVRLLNLRVGDAQGMFEPDGGGRPKGRLVAPLLATVTILDDDHAGIFSFQDRLLHVSECMGTVDVRVVRSSGARGTVRLPYRTVDGTARGGGVHYEDACGELEFGDDETMKTLQVKIVDDEEYEKKDNFFIELGQPQWLKRGISALLLNQGDGDRKLTAEEEEARRIAEMGKPVLGENCRLEVIIEESYDFKNTVDKLIKKTNLALVIGTHSWREQFLEAITVSAGDEEEEEDGSREERLPSCFDYVMHFLTVFWKVLFACVPPTEYCHGWACFGVSILVIGLLTALIGDLASHFGCTVGLKDSVNAVVFVALGTSIPDTFASKVAALQDQCADASIGNVTGSNAVNVFLGLGVAWSVAAVYWAVQGRPFEVRTGTLAFSVTLFTVFAFVGIAVLLYRRRPHIGGELGGPRGPKLATTALFLGLWLLYILFASLEAYCHIRGF

SEQ ID NO: 8 - DNA, 4282 bp, Homo Sapiens; identical to SEQ ID NO: 6
  CDS (94)...(2859)

SEQ ID NO: 9 - DNA, 2221 bp, Homo Sapiens
tgaaagaaccatctggttgagtctaaagttggaagatcaccatatccaccagaagacccaggatggatctgcaacaaagcaccaccatcacttccctagagaaatggtgtttggatgagtcactgtctggctgcagaagacattatagtgtcaagaaaaaactgaagttaattcgagtcttaggccttttcatgggtctggtagccattagcactgtctcattttcaatcagtgccttttctgagacagatacacagagcacaggagaggccagtgttgtaagtggccctagggtagcacagggttaccatcagagaactctcttagatttaaatgacaagattctggattatactccacagccacctctttctaaggaaggcgagtctgagaatagtacagatcacgcccaaggagactacccgaaagacatcttttcccttgaggagagaagaaaaggtgcgatcattctgcatgtcattggaatgatctacatgttcatagccttagccattgtctgtgatgagttctttgttccttctttgactgtcatcactgaaaaactgggcatctctgatgatgtggctggagccaccttcatggctgcaggagggtcagccccagaacttttcacatctctcataggggtatttatcgctcacagcaacgttggcataggcacaattgtaggttcagcagtattcaacatcctctttgttattggcatgtgtgctctgttttctagagaaatcttaaacctgacatggtggccgctctttcgagatgtgtctttctacattgttgacttgatcatgctgatcatatttttcctggataatgtcatcatgtggtgggaaagcttgcttctcttaacagcttatttttgctatgtggttttcatgaaattcaacgtccaagtagaaaaatgggtgaagcaaatgataaaccgcaataaggtcgtcaaggtgacagcaccagaagcccaagcaaagccatctgcagccagggacaaggatgaaccgactctaccggctaagccgcgtctccagcgaggtggaagctctgcctccctccacaacagtctcatgaggaatagcatcttccaactcatgatacacacccttgacccactcgccgaagaacttggatcatatggaaaactaaaatattatgacacaatgactgaagaagggaggttcagagaaaaggcttcaattctccacaagatcgccaagaagaaatgtcatgtggatgagaacgagcggcagaatggggctgccaaccacgtggaaaaaattgagcttccaaacagcaccagcacagatgttgaaatgacaccatccagtgatgcttcagaacctgtacaaaatggaaatctctcccacaacattgaaggtgcagaagcccagaccgctgatgaggaggaggaccagcctctcagccttgcctggccttctgaaacccgcaagcaagtcacgtttctgattgttttccccatagtgtttcctctctggattacgttacctgacgttcgcaaaccttcatcgaggaagttttttcccatcacgttctttggctccattacctggattgcagtattctcttacttgatggtctggtgggcgcaccaggttggagagacaattggcatcagtgaagagattatgggcctgaccatcttggctgctgggacctccatccctgatcttatcaccagtgtcatagtggcccggaaggggttaggggacatggctgtgtccagctctgttggaagcaacatttttgacatcactgtagggctcccactgccctggctcctgtacaccgtcattcacagattccagccagtggctgtcagcagcaatggccttttctgtgccatcgtccttctcttcatcatgctgctcttcgtcatcctctctatcgccctctgcaagtggcgaatgaacaaaatcctgggcttcatcatgtttggcctctactttgtgttcctggtggtgagcgttctcctagaagacagaattcttacatgccccgtctccatctagcaggaaaagccatatcttgcaccagcagcatgaatggtccctccacactctgggctctgggctccttgacctcttgagaagaggcagctggcacacagccctgggtgccaagtgtccctccttggtgaatttgaggagagatggattcacactgggcccattcacttcacagg

SEQ ID NO: 10 - protein, 661 aa, Homo Sapiens
MDLQQSTTITSLEKWCLDESLSGCRRHYSVKKKLKLIRVLGLFMGLVAISTVSFSISAFSETDTQSTGEASVVSGPRVAQGYHQRTLLDLNDKILDYTPQPPLSKEGESENSTDHAQGDYPKDIFSLEERRKGAIILHVIGMIYMFIALAIVCDEFFVPSLTVITEKLGISDDVAGATFMAAGGSAPELFTSLIGVFIAHSNVGIGTIVGSAVFNILFVIGMCALFSREILNLTWWPLFRDVSFYIVDLIMLIIFFLDNVIMWWESLLLLTAYFCYVVFMKFNVQVEKWVKQMINRNKVVKVTAPEAQAKPSAARDKDEPTLPAKPRLQRGGSSASLHNSLMRNSIFQLMIHTLDPLAEELGSYGKLKYYDTMTEEGRFREKASILHKIAKKKCHVDENERQNGAANHVEKIELPNSTSTDVEMTPSSDASEPVQNGNLSHNIEGAEAQTADEEEDQPLSLAWPSETRKQVTFLIVFPIVFPLWITLPDVRKPSSRKFFPITFFGSITWIAVFSYLMVWWAHQVGETIGISEEIMGLTILAAGTSIPDLITSVIVARKGLGDMAVSSSVGSNIFDITVGLPLPWLLYTVIHRFQPVAVSSNGLFCAIVLLFIMLLFVILSIALCKWRMNKILGFIMFGLYFVFLVVSVLLEDRILTCPVSI

SEQ ID NO: 11 - DNA, 2221 bp, Homo Sapiens; identical to SEQ ID NO: 9
  CDS (63)...(2048)

SEQ ID NO: 12 - DNA, 1911 bp, Homo Sapiens
atgaagaagctccagggagctcacctccgcaagcctgtcaccccagacctgctgatgacccccagtgaccagggcgatgtcgacctggatgtggactttgctgcacaccgggggaactggacaggcaagctggacttcctgctgtcctgcattggctactgtgtaggcctggggaatgtctggcgcttcccctatcgagcgtacaccaatggaggaggcgccttcctcgtgccctacttcctcatgctggccatctgtggcatccccctcttcttcctggagctctccctgggccagttctccagcctagggcccctggctgtctggaaaatcagccctctcttcaaaggcgccggcgcagccatgctgctcatcgtgggcttggtggccatctactacaacatgatcatcgcctacgtgctcttctacctcttcgcctccctcaccagcgacctaccctgggagcactgtggcaactggtggaacacagaactctgcctggagcacagagtctccaaggacggcaacggggccctgcccctcaacctcacctgcaccgtcagccccagtgaggagtactggagccgctacgtcctccacatccaaggcagccagggcatcggcagccctggggagatccgctggaacctctgcctctgcctgctgctggcctgggtcatcgtgttcctctgtatcctcaagggtgtgaagtcttcgggcaaggtggtgtatttcacggccacgttcccctacctcatcctgctcatgctgctggtccgcggagtcaccctcccaggggcctggaagggcatccagttctatctcaccccccagttccaccacttgttgtcttccaaggtgtggattgaagctgctcttcagatcttctattccctgggtgtgggcttcggggggctcctcacctttgcctcctacaacacgtttcaccagaacatctatagagacaccttcatcgtcactctgggcaacgccatcaccagcatcctggctggctttgccatcttctccgtgctgggctacatgtctcaggagctgggcgtgcctgtggaccaagtagccaaagcaggccctggcctggcctttgtcgtctacccacaggccatgaccatgctgcctctgtcacccttctggtcctttctcttcttcttcatgcttctgactctcggcctagatagccagtttgcctttctggagaccattgtgacagctgtgacagatgagttcccatactacctgcggcccaagaaggcggtgttctcagggctcatctgcgtggccatgtacctgatggggctgatcctcaccactgatgggggcatgtactggctggtccttctggatgactacagcgccagcttcgggctgatggtggtggttatcaccacatgccttgccgtgacacgggtgtatggcattcagaggttctgccgagacatccacatgatgctgggcttcaagccgggcctctacttcagggcctgctggctgttcctgtccccagccacgctcttggccctcctggtgtatagcatcgtcaagtaccagccctcggagtatggcagttaccgcttcccgccctgggctgagctgctgggcatcctgatgggcctgctgtcctgcctcatgatcccagctggcatgctggtggctgtgcttcgagaagagggctcactctgggagcggctccaacaggccagccggccggccatggactggggaccatcgctggaggagaaccggacgggcatgtatgtggccacgctggctgggagccagtcaccaaagccactgatggtgcacatgcgcaagtacgggggcatcaccagcttcgagaacacggccatcgaggtggaccgtgagattgcagaggaggaggagtcgatgatgtga

SEQ ID NO: 13 - DNA, 1911 bp, Homo Sapiens; identical to SEQ ID NO: 12
  CDS (1)...(1911)

SEQ ID NO: 14 - protein, 636 aa, Homo Sapiens
MKKLQGAHLRKPVTPDLLMTPSDQGDVDLDVDFAAHRGNWTGKLDFLLSCIGYCVGLGNVWRFPYRAYTNGGGAFLVPYFLMLAICGIPLFFLELSLGQFSSLGPLAVWKISPLFKGAGAAMLLIVGLVAIYYNMIIAYVLFYLFASLTSDLPWEHCGNWWNTELCLEHRVSKDGNGALPLNLTCTVSPSEEYWSRYVLHIQGSQGIGSPGEIRWNLCLCLLLAWVIVFLCILKGVKSSGKVVYFTATFPYLILLMLLVRGVTLPGAWKGIQFYLTPQFHHLLSSKVWIEAALQIFYSLGVGFGGLLTFASYNTFHQNIYRDTFIVTLGNAITSILAGFAIFSVLGYMSQELGVPVDQVAKAGPGLAFVVYPQAMTMLPLSPFWSFLFFFMLLTLGLDSQFAFLETIVTAVTDEFPYYLRPKKAVFSGLICVAMYLMGLILTTDGGMYWLVLLDDYSASFGLMVVVITTCLAVTRVYGIQRFCRDIHMMLGFKPGLYFRACWLFLSPATLLALLVYSIVKYQPSEYGSYRFPPWAELLGILMGLLSCLMIPAGMLVAVLREEGSLWERLQQASRPAMDWGPSLEENRTGMYVATLAGSQSPKPLMVHMRKYGGITSFENTAIEVDREIAEEEESMM

SEQ ID NO: 15 - DNA, 1635 bp, Homo Sapiens
atgtctgacggtctggataatgaagagaaacccccggctcctccactgaggatgaatagtaacaaccgggattcttcagcactcaaccacagctccaaaccacttcccatggcccctgaagagaagaataagaaagccaggcttcgctctatcttcccaggaggaggggataaaaccaataagaagaaggagaaagagcgcccagagatctctcttccttcagactttgagcatacgattcatgtggggtttgatgcagtcaccggggaattcactggaattccagagcaatgggcacgattactccaaacttccaacataacaaaattggaacagaagaagaacccacaagctgttctagatgttctcaaattctatgattccaaagaaacagtcaacaaccagaaatacatgagctttacatcaggagataaaagtgcacatggatacatagcagcccatccttcgagtacaaaaacagcatctgagcctccattggcccctcctgtgtctgaagaagaagatgaagaggaagaagaagaagaagatgaaaatgagccaccaccagttatcgcaccaagaccagagcatacaaaatcaatctatactcgttctgtggttgaatccattgcttcaccagcagtaccaaataaagaggtcacaccaccctctgctgaaaatgccaattccagtactttgtacaggaacacagatcggcaaagaaaaaaatccaagatgacagatgaggagatcttagagaagctaagaagcattgtgagtgttggggacccaaagaaaaaatacacaagatttgaaaaaattggtcaaggggcatcaggtactgtttatacagcactagacattgcaacaggacaagaggtggccataaagcagatgaaccttcaacagcaacccaagaaggaattaattattaatgaaattctggtcatgagggaaaataagaaccctaatattgttaattatttagatagctacttggtgggtgatgaactatgggtagtcatggaatacttggctggtggctctctgactgatgtggtcacagagacctgtatggatgaaggacagatagcagctgtctgcagagagtgcctgcaagctttggatttcctgcactcaaaccaggtgatccatagagatataaagagtgacaatattcttctcgggatggatggctctgttaaattgactgactttgggttctgtgcccagatcactcctgagcaaagtaaacgaagcactatggtgggaaccccatattggatggcacctgaggtggtgactcgaaaagcttatggtccgaaagttgatatctggtctcttggaattatggcaattgaaatggtggaaggtgaacccccttaccttaatgaaaatccactcagggcattgtatctgatagccactaatggaactccagagctccagaatcctgagagactgtcagctgtattccgtgactttttaaatcgctgtcttgagatggatgtggataggcgaggatctgccaaggagcttttgcagcatccatttttaaaattagccaagcctctctccagcctgactcctctgattatcgctgcaaaggaagcaattaagaacagcagccgctaa

SEQ ID NO: 16 - DNA, 1635 bp, Homo Sapiens; identical to SEQ ID NO: 15
  CDS (1)...(1635)

SEQ ID NO: 17 - protein, 544 aa, Homo Sapiens
MSDGLDNEEKPPAPPLRMNSNNRDSSALNHSSKPLPMAPEEKNKKARLRSIFPGGGDKTNKKKEKERPEISLPSDFEHTIHVGFDAVTGEFTGIPEQWARLLQTSNITKLEQKKNPQAVLDVLKFYDSKETVNNQKYMSFTSGDKSAHGYIAAHPSSTKTASEPPLAPPVSEEEDEEEEEEEDENEPPPVIAPRPEHTKSIYTRSVVESIASPAVPNKEVTPPSAENANSSTLYRNTDRQRKKSKMTDEEILEKLRSIVSVGDPKKKYTRFEKIGQGASGTVYTALDIATGQEVAIKQMNLQQQPKKELIINEILVMRENKNPNIVNYLDSYLVGDELWVVMEYLAGGSLTDVVTETCMDEGQIAAVCRECLQALDFLHSNQVIHRDIKSDNILLGMDGSVKLTDFGFCAQITPEQSKRSTMVGTPYWMAPEVVTRKAYGPKVDIWSLGIMAIEMVEGEPPYLNENPLRALYLIATNGTPELQNPERLSAVFRDFLNRCLEMDVDRRGSAKELLQHPFLKLAKPLSSLTPLIIAAKEAIKNSSR

SEQ ID NO: 18 - DNA, 2520 bp, Homo Sapiens
  misc_feature (1)...(2520): n = A,T,C or G
ccacgcgtccgggctttgtctcgtgggctggtccccagcggctccctccccgaacagctgctgctccagggaggaagcggcgcgggtgctgtccagcttcccggtgctgaaaaccggagggctcgtcatccaccactaccatgtaagggccatgagaagggctcatcctggcgcagcgcggacatggaggaggacttattccagctaaggcagctgccggttgtgaaattccgtcgcacaggcgagagtgcaaggtcagaggacgacacggcttcaggagagcatgaagtccagattgaaggggtccacgtgggcctagaggctgtggagctggatgatggggcagctgtgcccaaggagtttgccaatcccaccgatgatactttcatggtggaagatgcagtggaagccattggctttggaaaatttcagtggaagctgtctgttctcactggcttggcttggatggctgatgccatggagatgatgatcctcagcatcctggcaccacagctgcattgcgagtggaggctcccaagctggcaggtggcattgctgacctcggtggtctttgtaggcatgatgtccagctccacgctctggggaaatatctcagaccagtacggcaggaaaacagggctgaagatcagcgtgctgtggactctgtactatggcatccttagtgcatttgcgcccgtgtatagctggatcctggtgctccggggcctggtgggcttcgggatcggaggagttccccagtcggtgacgctgtatgccgagttccttcccatgaaagccagagctaaatgtattttgctgattgaggtattctgggccatcgggacagtgttcgaggtcgtcctggctgtgttcgtgatgcccagcctgggctggcgttggctgctcatcctctcagctgtcccgctcctcctctttgccgtgctgtgtttctggctgcctgaaagtgcaaggtatgatgtgctgtcagggaaccaggaaaaggcaatcgccaccttaaagaggatagcaactgaaaacggagctcccatgccgctggggaaactcatcatctccagacaggaagaccgaggcaaaatgagggaccttttcacaccccattttagatggacaactttgctgctgtggtttatatggttttccaatgcattctcttactacgggttagttctactcaccacagaactcttccaggcaggagatgtctgcggcatctccagtcggaagaaggctgtagaggcaaaatgcagcctggcctgcgagtacctgagtgaggaggattacatggacttgctgtggaccaccctctctgagtttccaggtgtccttgtgactctgtggattattgaccgcctggggcgcaagaagaccatggccctgtgctttgtcatcttctccttctgcagcctcctgctgtttatctgtgttggaagaaatgtgctcactctgttactcttcattgcaagagcgtttatttctggaggctttcaagcggcatatgtttacacacctgaggtctaccccacggcaacgcgggccctcggcctgggcacctgcagcggcatggcaagagtgggtgctctcatcactccgttcatcgcccaggtgatgctggaatcctctgtgtacctgactctggcagtttacagtggctgctgcctcctggctgccctggcctcctgctttttgcccattgagaccaaaggcggaggactgcaggagtccagccaccgggagtggggccaggagatggtcggccgaggaatgcacggtgcaggtgttaccaggtcgaactctggctctcaggaatagtgaccgatgggggactgagctggtctttgaggctgcagagcttggggggctggcaggccccaactggggcactgattgtcactgccgacatcaagaactcacccaagagtatgacctggaccaacagggttttgtgtcttgactcagtttgctcatcttcattgaggtccacccagggatggggagatgtttgctctagggggttctctgtatatgtggtgaaagctttgttcataacctgtggatctacatgggaagactacccatattaggagggtctggtaatgccagcaaccaatcagacaccacccagagtcacccggccaaaccctcagtgaacaaccaaaatatctctctgtagataccgtccaggctcaggcccatgtgacacctgctgtccacccaccggacctgttcagtaggtttctcccacacccacagccccaggctttcttctttgaaattgcaggcgatctaggtgtggtctgagcagctatttcctggcaggggccccccggtttgcctccctagagcctgaccagtggattctctggcagatggacatggtgcattcaaactggagccacatgcccccacccagcccctnttggagttgcccgttgttggcaccaagagatccagatgtgtcctggggacagctgggtcttgcaccaggtgacaacctcaaaa

SEQ ID NO: 19 - DNA, 2520 bp, Homo Sapiens; identical to SEQ ID NO: 18
  CDS (184)...(1830)
  misc_feature (1)...(2520): n = A,T,C or G

SEQ ID NO: 20 - protein, 548 aa, Homo Sapiens
MEEDLFQLRQLPVVKFRRTGESARSEDDTASGEHEVQIEGVHVGLEAVELDDGAAVPKEFANPTDDTFMVEDAVEAIGFGKFQWKLSVLTGLAWMADAMEMMILSILAPQLHCEWRLPSWQVALLTSVVFVGMMSSSTLWGNISDQYGRKTGLKISVLWTLYYGILSAFAPVYSWILVLRGLVGFGIGGVPQSVTLYAEFLPMKARAKCILLIEVFWAIGTVFEVVLAVFVMPSLGWRWLLILSAVPLLLFAVLCFWLPESARYDVLSGNQEKAIATLKRIATENGAPMPLGKLIISRQEDRGKMRDLFTPHFRWTTLLLWFIWFSNAFSYYGLVLLTTELFQAGDVCGISSRKKAVEAKCSLACEYLSEEDYMDLLWTTLSEFPGVLVTLWIIDRLGRKKTMALCFVIFSFCSLLLFICVGRNVLTLLLFIARAFISGGFQAAYVYTPEVYPTATRALGLGTCSGMARVGALITPFIAQVMLESSVYLTLAVYSGCCLLAALASCFLPIETKGGGLQESSHREWGQEMVGRGMHGAGVTRSNSGSQE

SEQ ID NO: 21 - DNA, 2113 bp, Homo Sapiens
gcagccgccgcggtcgggctgccccctcccctcgccccgaccgctcccctgctggtgagggcctgcgcaggcggcggcggcggcgcccttggtggcagtggtggcggcggagcagcctcccgggatcgtgtctggagctcgaggagagggtagtgcccggcgagctatgcacgggggcggcggcgtctcctcctgttccgcctcctcagtctcctcggtctcgcagaatccgccggcggcggcggcgccaggacatggagctcgagaacatcgtggccaactcgctgctgctgaaagcgcgtcaaggaggatatggcaaaaaaagtggtcgtagtaaaaaatggaaggagatactgacactgcctcctgtcagccagtgcagtgagcttagacattccattgaaaaggattatagcagtctttgtgacaagcaaccgataggaagacgtctcttcaggcagttctgtgataccaaacccactctaaagaggcacattgaattcttggatgcagtggcagaatatgaagttgccgatgatgaggaccgaagtgattgtggactgtcaatcttagatagattcttcaatgataagttggcagcccctttaccagaaatacctccagatgttgtgacagaatgtagattgggactgaaggaggagaacccttccaaaaaagcctttgaggaatgtactagagttgcccataactacctaagaggggaaccatttgaagaataccaagaaagctcatatttttctcagtttttacaatggaaatggctggaaaggcaacccgtaacaaagaacacatttagacattacagagttctaggaaaaggcggatttggagaggtttgcgcctgtcaagtgcgagccacaggaaaaatgtatgcctgcaaaaagctacaaaaaaaaagaataaagaagaggaaaggtgaagctatggctctaaatgagaaaagaattctggagaaagtgcaaagtagattcgtagttagtttagcctacgcttatgaaaccaaagatgccttgtgcttggtgctcaccattatgaatggaggggatttgaagtttcacatttacaacctgggcaatcccggctttgatgagcagagagccgttttctatgctgcagagctgtgttgcggcttggaagatttacagagggaaagaattgtatacagagacttgaagcctgagaatattctccttgatgatcgtggacacatccggatttcagacctcggtttggccacagagatcccagaaggacagagggttcgaggaagagttggaacagtcggctacatggcacctgaagttgtcaataatgaaaagtatacgtttagtcccgattggtggggacttggctgtctgatctatgaaatgattcagggacattctccattcaaaaaatacaaagagaaagtcaaatgggaggaggtcgatcaaagaatcaagaatgataccgaggagtattctgagaagttttcagaggatgccaaatctatctgcaggatgttactcaccaagaatccaagcaagcggctgggctgcaggggcgagggagcggctggggtgaagcagcaccccgtgttcaaggacatcaacttcaggaggctggaggcaaacatgctggagccccctttctgtcctgatcctcatgccgtttactgtaaggacgtcctggatatcgagcagttctcggcggtgaaagggatctacctggacaccgcagatgaagacttctatgctcggtttgctaccgggtgtgtctccatcccctggcagaatgagatgatcgaatccgggtgtttcaaagacatcaacaaaagtgaaagtgaggaagctttgccattagatctagacaagaacatacataccccggtttccagaccaaacagaggcttcttctatagactcttcagaagagggggctgcctgaccatggtccccagtgagaaggaagtggaacccaagcaatgctgagcaccccggtgcggaccacagagcagaccctggcgccaggaaggagcatgtgttagcgtctcgtcccacctggaattgtaataaatacatctaaataaaacatgccttgggagtgtacagac

SEQ ID NO: 22 - DNA, 1737 bp, Homo Sapiens
  CDS (1)...(1737)
atggagctcgagaacatcgtggccaactcgctgctgctgaaagcgcgtcaaggaggatatggcaaaaaaagtggtcgtagtaaaaaatggaaggagatactgacactgcctcctgtcagccagtgcagtgagcttagacattccattgaaaaggattatagcagtctttgtgacaagcaaccgataggaagacgtctcttcaggcagttctgtgataccaaacccactctaaagaggcacattgaattcttggatgcagtggcagaatatgaagttgccgatgatgaggaccgaagtgattgtggactgtcaatcttagatagattcttcaatgataagttggcagcccctttaccagaaatacctccagatgttgtgacagaatgtagattgggactgaaggaggagaacccttccaaaaaagcctttgaggaatgtactagagttgcccataactacctaagaggggaaccatttgaagaataccaagaaagctcatatttttctcagtttttacaatggaaatggctggaaaggcaacccgtaacaaagaacacatttagacattacagagttctaggaaaaggcggatttggagaggtttgcgcctgtcaagtgcgagccacaggaaaaatgtatgcctgcaaaaagctacaaaaaaaaagaataaagaagaggaaaggtgaagctatggctctaaatgagaaaagaattctggagaaagtgcaaagtagattcgtagttagtttagcctacgcttatgaaaccaaagatgccttgtgcttggtgctcaccattatgaatggaggggatttgaagtttcacatttacaacctgggcaatcccggctttgatgagcagagagccgttttctatgctgcagagctgtgttgcggcttggaagatttacagagggaaagaattgtatacagagacttgaagcctgagaatattctccttgatgatcgtggacacatccggatttcagacctcggtttggccacagagatcccagaaggacagagggttcgaggaagagttggaacagtcggctacatggcacctgaagttgtcaataatgaaaagtatacgtttagtcccgattggtggggacttggctgtctgatctatgaaatgattcagggacattctccattcaaaaaatacaaagagaaagtcaaatgggaggaggtcgatcaaagaatcaagaatgataccgaggagtattctgagaagttttcagaggatgccaaatctatctgcaggatgttactcaccaagaatccaagcaagcggctgggctgcaggggcgagggagcggctggggtgaagcagcaccccgtgttcaaggacatcaacttcaggaggctggaggcaaacatgctggagccccctttctgtcctgatcctcatgccgtttactgtaaggacgtcctggatatcgagcagttctcggcggtgaaagggatctacctggacaccgcagatgaagacttctatgctcggtttgctaccgggtgtgtctccatcccctggcagaatgagatgatcgaatccgggtgtttcaaagacatcaacaaaagtgaaagtgaggaagctttgccattagatctagacaagaacatacataccccggtttccagaccaaacagaggcttcttctatagactcttcagaagagggggctgcctgaccatggtccccagtgagaaggaagtggaacccaagcaatgctga

SEQ ID NO: 23 - protein, 578 aa, Homo Sapiens
MELENIVANSLLLKARQGGYGKKSGRSKKWKEILTLPPVSQCSELRHSIEKDYSSLCDKQPIGRRLFRQFCDTKPTLKRHIEFLDAVAEYEVADDEDRSDCGLSILDRFFNDKLAAPLPEIPPDVVTECRLGLKEENPSKKAFEECTRVAHNYLRGEPFEEYQESSYFSQFLQWKWLERQPVTKNTFRHYRVLGKGGFGEVCACQVRATGKMYACKKLQKKRIKKRKGEAMALNEKRILEKVQSRFVVSLAYAYETKDALCLVLTIMNGGDLKFHIYNLGNPGFDEQRAVFYAAELCCGLEDLQRERIVYRDLKPENILLDDRGHIRISDLGLATEIPEGQRVRGRVGTVGYMAPEVVNNEKYTFSPDWWGLGCLIYEMIQGHSPFKKYKEKVKWEEVDQRIKNDTEEYSEKFSEDAKSICRMLLTKNPSKRLGCRGEGAAGVKQHPVFKDINFRRLEANMLEPPFCPDPHAVYCKDVLDIEQFSAVKGIYLDTADEDFYARFATGCVSIPWQNEMIESGCFKDINKSESEEALPLDLDKNIHTPVSRPNRGFFYRLFRRGGCLTMVPSEKEVEPKQC

SEQ ID NO: 24 - DNA, 1242 bp, Homo Sapiens
aagaggctctctctgcccatggatatccgcctgccccaggaattcctacagaagctacagatggagagcccagatctgcccaagccgctcagccgcatgtcccgccgggcctccctgtcagacattggctttgggaaactggaaacatacgtgaaactggacaaactgggagagggcacctatgccacagtcttcaaagggcgcagcaaactgacggagaaccttgtggccctgaaagagatccggctggagcacgaggagggagcgccctgcactgccatccgagaggtgtctctgctgaagaacctgaagcacgccaatattgtgaccctgcatgacctcatccacacagatcggtccctcaccctggtgtttgagtacctggacagtgacctgaagcagtatctggaccactgtgggaacctcatgagcatgcacaacgtcaagattttcatgttccagctgctccggggcctcgcctactgtcacacccgcaagatcctgcaccgggacctgaagccccagaacctgctcatcaacgagaggggggagctgaagctggccgactttggactggccagggccaagtcagtgcccacaaagacttactccaatgaggtggtgaccctgtggtacaggccccccgatgtgctgctgggatccacagagtactccacccccattgctatgtggggcgtgggctgcatccactacgagatggccacagggaggcccctcttcccgggctccacagtcaaggaggagctgcacctcatctttcgcctcctcgggacccccacagaagagacgtggcccggcgtgaccgccttctctgagttccgcacctacagcttcccctgctacctcccgcagccgctcatcaaccacgcgcccaggttggatacggatggcatccacctcctgagcagcctgctcgtgtatgaatccaagagtcgcatgtcagcagaggctgccctgagtcactcctacttccggtctctgggagagcgtgtgcaccagcttgaagacactgcctccatcttctccctgaaggagatccagctccagaaggacccaggctaccgaggcttggccttccagcagccaggacgagggaagaacaggcggcagagcatcttctgagccacgcccaccttgctgtggccaagggacaagagaccacatggagcacaaattcgggtaggatggagcctgtgtggccctcggaggactgaacaacga

SEQ ID NO: 25 - DNA, 1125 bp, Homo Sapiens
  CDS (1)...(1125)
atggatatccgcctgccccaggaattcctacagaagctacagatggagagcccagatctgcccaagccgctcagccgcatgtcccgccgggcctccctgtcagacattggctttgggaaactggaaacatacgtgaaactggacaaactgggagagggcacctatgccacagtcttcaaagggcgcagcaaactgacggagaaccttgtggccctgaaagagatccggctggagcacgaggagggagcgccctgcactgccatccgagaggtgtctctgctgaagaacctgaagcacgccaatattgtgaccctgcatgacctcatccacacagatcggtccctcaccctggtgtttgagtacctggacagtgacctgaagcagtatctggaccactgtgggaacctcatgagcatgcacaacgtcaagattttcatgttccagctgctccggggcctcgcctactgtcacacccgcaagatcctgcaccgggacctgaagccccagaacctgctcatcaacgagaggggggagctgaagctggccgactttggactggccagggccaagtcagtgcccacaaagacttactccaatgaggtggtgaccctgtggtacaggccccccgatgtgctgctgggatccacagagtactccacccccattgctatgtggggcgtgggctgcatccactacgagatggccacagggaggcccctcttcccgggctccacagtcaaggaggagctgcacctcatctttcgcctcctcgggacccccacagaagagacgtggcccggcgtgaccgccttctctgagttccgcacctacagcttcccctgctacctcccgcagccgctcatcaaccacgcgcccaggttggatacggatggcatccacctcctgagcagcctgctcgtgtatgaatccaagagtcgcatgtcagcagaggctgccctgagtcactcctacttccggtctctgggagagcgtgtgcaccagcttgaagacactgcctccatcttctccctgaaggagatccagctccagaaggacccaggctaccgaggcttggccttccagcagccaggacgagggaagaacaggcggcagagcatcttctga

SEQ ID NO: 26 - protein, 374 aa, Homo Sapiens
MDIRLPQEFLQKLQMESPDLPKPLSRMSRRASLSDIGFGKLETYVKLDKLGEGTYATVFKGRSKLTENLVALKEIRLEHEEGAPCTAIREVSLLKNLKHANIVTLHDLIHTDRSLTLVFEYLDSDLKQYLDHCGNLMSMHNVKIFMFQLLRGLAYCHTRKILHRDLKPQNLLINERGELKLADFGLARAKSVPTKTYSNEVVTLWYRPPDVLLGSTEYSTPIAMWGVGCIHYEMATGRPLFPGSTVKEELHLIFRLLGTPTEETWPGVTAFSEFRTYSFPCYLPQPLINHAPRLDTDGIHLLSSLLVYESKSRMSAEAALSHSYFRSLGERVHQLEDTASIFSLKEIQLQKDPGYRGLAFQQPGRGKNRRQSIF

SEQ ID NO: 27 - DNA, 4151 bp, Homo Sapiens
cgcaacttcatagacggttgtgaagacaagctagacggcagatgaaagttcttggcacagagtgaacacttgataaactatatggcagccacagcctccggagccgttgcacacctacctgcccggccgacttacctgtacttgccgccgtcccggctcacctggcggtgcccgaggagtagtcgctggagtccgcgcctccctgggactgcaatgtgccgatcttagctgctgcctgagaggatgtctggggtgtccgagcccctgagtcgagtaaagttgggcacgttacgccggcctgaaggccctggagagcccatggtggtggtaccagtagatgtggaaaaggaggacgtgcgtatcctcaaggtctgcttctatagcaacagcttcaatcctgggaaaaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacagtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccggcttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatctacgcagagattcccgacgaaaccctgcgaaggcccggaggtccacagtatggcattgcccgtgaagatgtggtcctgaatcgtattcttggggaaggcctttttggggaggtctatgaaggtgtctacacaaatcataaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgcgggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggaccgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcgcagcagaacgccgtgacctccctgagtgaggagtgcaagaggcagatgctgacggcttcacacaccctggctgtggacgccaagaacctgctcgacgctgtggaccaggccaaggttctggccaatctggcccacccacctgcagagtgacggagggtggggccacctgcctgcgtcttcgcccctgcctgccatgtacctcccctgccttgctgttggtcatgtgggtcttccagggagaaggccaaggggagtcaccttcccttgccactttgcacgacgccctctccccacccctacccctggctgtactgctcaggctgcagctggacagaggggactctgggctatggacacagggtgacggtgacaaagatggctcagagggggactgctgctgcctggccactgctccctaagccagcctggtccatgcagggggctcctgggggtggggaggtgtcacatggtgcccctagctttatatatggacatggcaggccgatttgggaaccaagctattcctttcccttcctcttcggccctcagatgtcccttgatgcacagagaagctggggaggagctttgttttgggggtcaggcagccagtgagatgagggatgggcctggcattcttgtacagtgtatattgaaatttatttaatgtgagtttggtctggactgacagcatgtgccctcctgagggaggacctggggcacagtccaggaacaagctaattgggagtccaggcacaggatgctgtgttgtcaacaaaccaagcatcagggggaagaagcagagagatgcggccaagataggaccttgggccaaatccgctctcttcctgcccctctttctctttcttcctttactttcccttgcttttccctcttttcttactcctcctctttctctccccaacccccattctcatctgcacccttcttttctcatgtgtttgcataaacattcttttaacttctttctatttgacttgtggttgaattaaaattgtcccatttgcttg

SEQ ID NO: 28 - DNA, 3030 bp, Homo Sapiens
  CDS (1)...(3030)
atgtctggggtgtccgagcccctgagtcgagtaaagttgggcacgttacgccggcctgaaggccctggagagcccatggtggtggtaccagtagatgtggaaaaggaggacgtgcgtatcctcaaggtctgcttctatagcaacagcttcaatcctgggaaaaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacagtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccggcttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatctacgcagagattcccgacgaaaccctgcgaaggcccggaggtccacagtatggcattgcccgtgaagatgtggtcctgaatcgtattcttggggaaggcctttttggggaggtctatgaaggtgtctacacaaatcataaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgcgggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggaccgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcgcagcagaacgccgtgacctccctgagtgaggagtgcaagaggcagatgctgacggcttcacacaccctggctgtggacgccaagaacctgctcgacgctgtggaccaggccaaggttctggccaatctggcccacccacctgcagagtga

SEQ ID NO: 29 - protein, 1009 aa, Homo Sapiens [sequence omitted]

SEQ ID NO: 30 - DNA, 6574 bp, Homo Sapiens
agagccgccagtggggagatgttgaagttcaaatatggagcgcggaatcctttggatgctggtgctgctgaacccattgccagccgggcctccaggctgaatctgttcttccaggggaaaccaccctttatgactcaacagcagatgtctcctctttcccgagaagggatattagatgccctctttgttctctttgaagaatgcagtcagcctgctctgatgaagattaagcacgtgagcaactttgtccggaagtattccgacaccatagctgagttacaggagctccagccttcggcaaaggacttcgaagtcagaagtcttgtaggttgtggtcactttgctgaagtgcaggtggtaagagagaaagcaaccggggacatctatgctatgaaagtgatgaagaagaaggctttattggcccaggagcaggtttcattttttgaggaagagcggaacatattatctcgaagcacaagcccgtggatcccccaattacagtatgcctttcaggacaaaaatcacctttatctgatggaggaatatcagcctggaggggacttgctgtcacttttgaatagatatgaggaccagttagatgaaaacctgatacagttttacctagctgagctgattttggctgttcacagcgttcatctgatgggatacgtgcatcgagacatcaagcctgagaacattctcgttgaccgcacaggacacatcaagctggtggattttggatctgccgcgaaaatgaattcaaacaagatggtgaatgccaaactcccgattgggaccccagattacatggctcctgaagtgctgactgtgatgaacggggatggaaaaggcacctacggcctggactgtgactggtggtcagtgggcgtgattgcctatgagatgatttatgggagatcccccttcgcagagggaacctctgccagaaccttcaataacattatgaatttccagcggtttttgaaatttccagatgaccccaaagtgagcagtgactttcttgatctgattcaaagcttgttgtgcggccagaaagagagactgaagtttgaaggtctttgctgccatcctttcttctctaaaattgactggaacaacattcgtaactctcctccccccttcgttcccaccctcaagtctgacgatgacacctccaattttgatgaaccagagaagaattcgtgggtttcatcctctccgtgccagctgagcccctcaggcttctcgggtgaagaactgccgtttgtggggttttcgtacagcaaggcactggggattcttggtagatctgagtctgttgtgtcgggtctggactcccctgccaagactagctccatggaaaagaaacttctcatcaaaagcaaagagctacaagactctcaggacaagtgtcacaagatggagcaggaaatgacccggttacatcggagagtgtcagaggtggaggctgtgcttagtcagaaggaggtggagctgaaggcctctgagactcagagatccctcctggagcaggaccttgctacctacatcacagaatgcagtagcttaaagcgaagtttggagcaagcacggatggaggtgtcccaggaggatgacaaagcactgcagcttctccatgatatcagagagcagagccggaagctccaagaaatcaaagagcaggagtaccaggctcaagtggaagaaatgaggttgatgatgaatcagttggaagaggatcttgtctcagcaagaagacggagtgatctctacgaatctgagctgagagagtctcggcttgctgctgaagaattcaagcggaaagcgacagaatgtcagcataaactgttgaaggctaaggatcaagggaagcctgaagtgggagaatatgcgaaactggagaagatcaatgctgagcagcagctcaaaattcaggagctccaagagaaactggagaaggctgcaaaggagcgagccgagagggagctggagaagctgcagaaccgagaggattcttctgaaggcatcagaaagaagctggtggaagctgaggaacgccgccattctctggagaacaaggtaaagagactagagaccatggagcgtagagaaaacagactgaaggatgacatccagacaaaatcccaacagatccagcagatggctgataaaattctggagctcgaagagaaacatcgggaggcccaagtctcagcccagcacctagaagtgcacctgaaacagaaagagcagcactatgaggaaaagattaaagtgttggacaatcagataaagaaagacctggctgacaaggagacactggagaacatgatgcagagacacgaggaggaggcccatgagaagggcaaaattctcagcgaacagaaggcgatgatcaatgctatggattccaagatcagatccctggaacagaggattgtggaactgtctgaagccaataaacttgcagcaaatagcagtctttttacccaaaggaacatgaaggcccaagaagagatgatttctgaactcaggcaacagaaattttacctggagacacaggctgggaagttggaggcccagaaccgaaaactggaggagcagctggagaagatcagccaccaagaccacagtgacaagaatcggctgctggaactggagacaagattgcgggaggtcagtctagagcacgaggagcagaaactggagctcaagcgccagctcacagagctacagctctccctgcaggagcgcgagtcacagttgacagccctgcaggctgcacgggcggccctggagagccagcttcgccaggcgaagacagagctggaagagaccacagcagaagctgaagaggagatccaggcactcacggcacatagagatgaaatccagcgcaaatttgatgctcttcgtaacagctgtactgtaatcacagacctggaggagcagctaaaccagctgaccgaggacaacgctgaactcaacaaccaaaacttctacttgtccaaacaactcgatgaggcttctggcgccaacgacgagattgtacaactgcgaagtgaagtggaccatctccgccgggagatcacggaacgagagatgcagcttaccagccagaagcaaacgatggaggctctgaagaccacgtgcaccatgctggaggaacaggtcatggatttggaggccctaaacgatgagctgctagaaaaagagcggcagtgggaggcctggaggagcgtcctgggtgatgagaaatcccagtttgagtgtcgggttcgagagctgcagagaatgctggacaccgagaaacagagcagggcgagagccgatcagcggatcaccgagtctcgccaggtggtggagctggcagtgaaggagcacaaggctgagattctcgctctgcagcaggctctcaaagagcagaagctgaaggccgagagcctctctgacaagctcaatgacctggagaagaagcatgctatgcttgaaatgaatgcccgaagcttacagcagaagctggagactgaacgagagctcaaacagaggcttctggaagagcaagccaaattacagcagcagatggacctgcagaaaaatcacattttccgtctgactcaaggactgcaagaagctctagatcgggctgatctactgaagacagaaagaagtgacttggagtatcagctggaaaacattcaggttctctattctcatgaaaaggtgaaaatggaaggcactatttctcaacaaaccaaactcattgattttctgcaagccaaaatggaccaacctgctaaaaagaaaaagggtttatttagtcgacggaaagaggaccctgctttacccacacaggttcctctgcagtacaatgagctgaagctggccctggagaaggagaaagctcgctgtgcagagctagaggaagcccttcagaagacccgcatcgagctccggtccgcccgggaggaagctgcccaccgcaaagcaacggaccacccacacccatccacgccagccaccgcgaggcagcagatcgccatgtccgccatcgtgcggtcgccagagcaccagcccagtgccatgagcctgctggccccgccatccagccgcagaaaggagtcttcaactccagaggaatttagtcggcgtcttaaggaacgcatgcaccacaatattcctcaccgattcaacgtaggactgaacatgcgagccacaaagtgtgctgtgtgtctggataccgtgcactttggacgccaggcatccaaatgtctcgaatgtcaggtgatgtgtcaccccaagtgctccacgtgcttgccagccacctgcggcttgcctgctgaatatgccacacacttcaccgaggccttctgccgtgacaaaatgaactccccaggtctccagaccaaggagcccagcagcagcttgcacctggaagggtggatgaaggtgcccaggaataacaaacgaggacagcaaggctgggacaggaagtacattgtcctggagggatcaaaagtcctcatttatgacaatgaagccagagaagctggacagaggccggtggaagaatttgagctgtgccttcccgacggggatgtatctattcatggtgccgttggtgcttccgaactcgcaaatacagccaaagcagaaaaagcagaagctgatgctaaactgcttggaaactccctgctgaaactggaaggtgatgaccgtctagacatgaactgcacgctgcccttcagtgaccaggtggtgttggtgggcaccgaggaagggctctacgccctgaatgtcttgaaaaactccctaacccatgtcccaggaattggagcagtcttccaaatttatattatcaaggacctggagaagctactcatgatagcaggagaagagcgggcactgtgtcttgtggacgtgaagaaagtgaaacagtccctggcccagtcccacctgcctgcccagcccgacatctcacccaacatttttgaagctgtcaagggctgccacttgtttggggcaggcaagattgagaacgggctctgcatctgtgcagccatgcccagcaaagtcgtcattctccgctacaacgaaaacctcagcaaatactgcatccggaaagagatagagacctcagagccctgcagctgtatccacttcaccaattacagtatcctcattggaaccaataaattctacgaaatcgacatgaagcagtacacgctcgaggaattcctggataagaatgaccattccttggcacctgctgtgtttgccgcctcttccaacagcttccctgtctcaatcgtgcaggtgaacagcgcagggcagcgagaggagtacttgctgtgtttccacgaatttggagtgttcgtggattcttacggaagacgtagccgcacagacgatctcaagtggagtcgcttacctttggcctttgcctacagagaaccctatctgtttgtgacccacttcaactcactcgaagtaattgagatccaggcacgctcctcagcagggacccctgcccgagcgtacctggacatcccgaacccgcgctacctgggccctgccatttcctcaggagcgatttacttggcgtcctcataccaggataaattaagggtcatttgctgcaagggaaacctcgtgaaggagtccggcactgaacaccaccggggcccgtccacctcccgcagcagccccaacaagcgaggcccacccacgtacaacgagcacatcaccaagcgcgtggcctccagcccagcgccgcccgaaggccccagccacccgcgagagccaagcacaccccaccgctaccgcgaggggcggaccgagctgcgcagggacaagtctcctggccgccccctggagcgagagaagtcccccggccggatgctcagcacgcggagagagcggtcccccgggaggctgtttgaagacagcagcaggggccggctgcctgcgggagccgtgaggaccccgctgtcccaggtgaacaagggaagagggcagagtgcctctcaagttttcacggttaacactgtcacctattatgactggaataaaaagctggacaacctgccagctaactggtcagtcctgaggatcatccagctgaatggagaaatccggcagcaggttgaaaagtctgttctgagaacagattattgctgagcagagttcatgtgacttctagacgtggtgacttaaaaaatggccttaaggctgcagagccagccacctctgcttacaaaaagagtacttagtgcacatgactgtaagaaacaattgtaaaacctcatctagaaatcagaaagcttctaatttctatagaaatgacacctccctggagccgagagacaatctgttgttgattttgaaggacaggcaagaccaacactgtatttagttccatagccaggcctcaacagggacaagtggctggccttaaaaacacacagatgactggaaatgatgtgtggcctcagtccctgtttcccagaattttactggcaaaggagttagcattcatttttggcttaagaaaaatcgagaatgtaggtttaga

SEQ ID NO: 31 - DNA, 6162 bp, Homo Sapiens
  CDS (1)...(6162)
atgttgaagttcaaatatggagcgcggaatcctttggatgctggtgctgctgaacccattgccagccgggcctccaggctgaatctgttcttccaggggaaaccaccctttatgactcaacagcagatgtctcctctttcccgagaagggatattagatgccctctttgttctctttgaagaatgcagtcagcctgctctgatgaagattaagcacgtgagcaactttgtccggaagtattccgacaccatagctgagttacaggagctccagccttcggcaaaggacttcgaagtcagaagtcttgtaggttgtggtcactttgctgaagtgcaggtggtaagagagaaagcaaccggggacatctatgctatgaaagtgatgaagaagaaggctttattggcccaggagcaggtttcattttttgaggaagagcggaacatattatctcgaagcacaagcccgtggatcccccaattacagtatgcctttcaggacaaaaatcacctttatctgatggaggaatatcagcctggaggggacttgctgtcacttttgaatagatatgaggaccagttagatgaaaacctgatacagttttacctagctgagctgattttggctgttcacagcgttcatctgatgggatacgtgcatcgagacatcaagcctgagaacattctcgttgaccgcacaggacacatcaagctggtggattttggatctgccgcgaaaatgaattcaaacaagatggtgaatgccaaactcccgattgggaccccagattacatggctcctgaagtgctgactgtgatgaacggggatggaaaaggcacctacggcctggactgtgactggtggtcagtgggcgtgattgcctatgagatgatttatgggagatcccccttcgcagagggaacctctgccagaaccttcaataacattatgaatttccagcggtttttgaaatttccagatgaccccaaagtgagcagtgactttcttgatctgattcaaagcttgttgtgcggccagaaagagagactgaagtttgaaggtctttgctgccatcctttcttctctaaaattgactggaacaacattcgtaactctcctccccccttcgttcccaccctcaagtctgacgatgacacctccaattttgatgaaccagagaagaattcgtgggtttcatcctctccgtgccagctgagcccctcaggcttctcgggtgaagaactgccgtttgtggggttttcgtacagcaaggcactggggattcttggtagatctgagtctgttgtgtcgggtctggactcccctgccaagactagctccatggaaaagaaacttctcatcaaaagcaaagagctacaagactctcaggacaagtgtcacaagatggagcaggaaatgacccggttacatcggagagtgtcagaggtggaggctgtgcttagtcagaaggaggtggagctgaaggcctctgagactcagagatccctcctggagcaggaccttgctacctacatcacagaatgcagtagcttaaagcgaagtttggagcaagcacggatggaggtgtcccaggaggatgacaaagcactgcagcttctccatgatatcagagagcagagccggaagctccaagaaatcaaagagcaggagtaccaggctcaagtggaagaaatgaggttgatgatgaatcagttggaagaggatcttgtctcagcaagaagacggagtgatctctacgaatctgagctgagagagtctcggcttgctgctgaagaattcaagcggaaagcgacagaatgtcagcataaactgttgaaggctaaggatcaagggaagcctgaagtgggagaatatgcgaaactggagaagatcaatgctgagcagcagctcaaaattcaggagctccaagagaaactggagaaggctgcaaaggagcgagccgagagggagctggagaagctgcagaaccgagaggattcttctgaaggcatcagaaagaagctggtggaagctgaggaacgccgccattctctggagaacaaggtaaagagactagagaccatggagcgtagagaaaacagactgaaggatgacatccagacaaaatcccaacagatccagcagatggctgataaaattctggagctcgaagagaaacatcgggaggcccaagtctcagcccagcacctagaagtgcacctgaaacagaaagagcagcactatgaggaaaagattaaagtgttggacaatcagataaagaaagacctggctgacaaggagacactggagaacatgatgcagagacacgaggaggaggcccatgagaagggcaaaattctcagcgaacagaaggcgatgatcaatgctatggattccaagatcagatccctggaacagaggattgtggaactgtctgaagccaataaacttgcagcaaatagcagtctttttacccaaaggaacatgaaggcccaagaagagatgatttctgaactcaggcaacagaaattttacctggagacacaggctgggaagttggaggcccagaaccgaaaactggaggagcagctggagaagatcagccaccaagaccacagtgacaagaatcggctgctggaactggagacaagattgcgggaggtcagtctagagcacgaggagcagaaactggagctcaagcgccagctcacagagctacagctctccctgcaggagcgcgagtcacagttgacagccctgcaggctgcacgggcggccctggagagccagcttcgccaggcgaagacagagctggaagagaccacagcagaagctgaagaggagatccaggcactcacggcacatagagatgaaatccagcgcaaatttgatgctcttcgtaacagctgtactgtaatcacagacctggaggagcagctaaaccagctgaccgaggacaacgctgaactcaacaaccaaaacttctacttgtccaaacaactcgatgaggcttctggcgccaacgacgagattgtacaactgcgaagtgaagtggaccatctccgccgggagatcacggaacgagagatgcagcttaccagccagaagcaaacgatggaggctctgaagaccacgtgcaccatgctggaggaacaggtcatggatttggaggccctaaacgatgagctgctagaaaaagagcggcagtgggaggcctggaggagcgtcctgggtgatgagaaatcccagtttgagtgtcgggttcgagagctgcagagaatgctggacaccgagaaacagagcagggcgagagccgatcagcggatcaccgagtctcgccaggtggtggagctggcagtgaaggagcacaaggctgagattctcgctctgcagcaggctctcaaagagcagaagctgaaggccgagagcctctctgacaagctcaatgacctggagaagaagcatgctatgcttgaaatgaatgcccgaagcttacagcagaagctggagactgaacgagagctcaaacagaggcttctggaagagcaagccaaattacagcagcagatggacctgcagaaaaatcacattttccgtctgactcaaggactgcaagaagctctagatcgggctgatctactgaagacagaaagaagtgacttggagtatcagctggaaaacattcaggttctctattctcatgaaaaggtgaaaatggaaggcactatttctcaacaaaccaaactcattgattttctgcaagccaaaatggaccaacctgctaaaaagaaaaagggtttatttagtcgacggaaagaggaccctgctttacccacacaggttcctctgcagtacaatgagctgaagctggccctggagaaggagaaagctcgctgtgcagagctagaggaagcccttcagaagacccgcatcgagctccggtccgcccgggaggaagctgcccaccgcaaagcaacggaccacccacacccatccacgccagccaccgcgaggcagcagatcgccatgtccgccatcgtgcggtcgccagagcaccagcccagtgccatgagcctgctggccccgccatccagccgcagaaaggagtcttcaactccagaggaatttagtcggcgtcttaaggaacgcatgcaccacaatattcctcaccgattcaacgtaggactgaacatgcgagccacaaagtgtgctgtgtgtctggataccgtgcactttggacgccaggcatccaaatgtctcgaatgtcaggtgatgtgtcaccccaagtgctccacgtgcttgccagccacctgcggcttgcctgctgaatatgccacacacttcaccgaggccttctgccgtgacaaaatgaactccccaggtctccagaccaaggagcccagcagcagcttgcacctggaagggtggatgaaggtgcccaggaataacaaacgaggacagcaaggctgggacaggaagtacattgtcctggagggatcaaaagtcctcatttatgacaatgaagccagagaagctggacagaggccggtggaagaatttgagctgtgccttcccgacggggatgtatctattcatggtgccgttggtgcttccgaactcgcaaatacagccaaagcagaaaaagcagaagctgatgctaaactgcttggaaactccctgctgaaactggaaggtgatgaccgtctagacatgaactgcacgctgcccttcagtgaccaggtggtgttggtgggcaccgaggaagggctctacgccctgaatgtcttgaaaaactccctaacccatgtcccaggaattggagcagtcttccaaatttatattatcaaggacctggagaagctactcatgatagcaggagaagagcgggcactgtgtcttgtggacgtgaagaaagtgaaacagtccctggcccagtcccacctgcctgcccagcccgacatctcacccaacatttttgaagctgtcaagggctgccacttgtttggggcaggcaagattgagaacgggctctgcatctgtgcagccatgcccagcaaagtcgtcattctccgctacaacgaaaacctcagcaaatactgcatccggaaagagatagagacctcagagccctgcagctgtatccacttcaccaattacagtatcctcattggaaccaataaattctacgaaatcgacatgaagcagtacacgctcgaggaattcctggataagaatgaccattccttggcacctgctgtgtttgccgcctcttccaacagcttccctgtctcaatcgtgcaggtgaacagcgcagggcagcgagaggagtacttgctgtgtttccacgaatttggagtgttcgtggattcttacggaagacgtagccgcacagacgatctcaagtggagtcgcttacctttggcctttgcctacagagaaccctatctgtttgtgacccacttcaactcactcgaagtaattgagatccaggcacgctcctcagcagggacccctgcccgagcgtacctggacatcccgaacccgcgctacctgggccctgccatttcctcaggagcgatttacttggcgtcctcataccaggataaattaagggtcatttgctgcaagggaaacctcgtgaaggagtccggcactgaacaccaccggggcccgtccacctcccgcagcagccccaacaagcgaggcccacccacgtacaacgagcacatcaccaagcgcgtggcctccagcccagcgccgcccgaaggccccagccacccgcgagagccaagcacaccccaccgctaccgcgaggggcggaccgagctgcgcagggacaagtctcctggccgccccctggagcgagagaagtcccccggccggatgctcagcacgcggagagagcggtcccccgggaggctgtttgaagacagcagcaggggccggctgcctgcgggagccgtgaggaccccgctgtcccaggtgaacaagggaagagggcagagtgcctctcaagttttcacggttaacactgtcacctattatgactggaataaaaagctggacaacctgccagctaactggtcagtcctgaggatcatccagctgaatggagaaatccggcagcaggttgaaaagtctgttctgagaacagattattgctga

SEQ ID NO: 32 - protein, 2053 aa, Homo Sapiens
MLKFKYGARNPLDAGAAEPIASRASRLNLFFQGKPPFMTQQQMSPLSREGILDALFVLFEECSQPALMKIKHVSNFVRKYSDTIAELQELQPSAKDFEVRSLVGCGHFAEVQVVREKATGDIYAMKVMKKKALLAQEQVSFFEEERNILSRSTSPWIPQLQYAFQDKNHLYLMEEYQPGGDLLSLLNRYEDQLDENLIQFYLAELILAVHSVHLMGYVHRDIKPENILVDRTGHIKLVDFGSAAKMNSNKMVNAKLPIGTPDYMAPEVLTVMNGDGKGTYGLDCDWWSVGVIAYEMIYGRSPFAEGTSARTFNNIMNFQRFLKFPDDPKVSSDFLDLIQSLLCGQKERLKFEGLCCHPFFSKIDWNNIRNSPPPFVPTLKSDDDTSNFDEPEKNSWVSSSPCQLSPSGFSGEELPFVGFSYSKALGILGRSESVVSGLDSPAKTSSMEKKLLIKSKELQDSQDKCHKMEQEMTRLHRRVSEVEAVLSQKEVELKASETQRSLLEQDLATYITECSSLKRSLEQARMEVSQEDDKALQLLHDIREQSRKLQEIKEQEYQAQVEEMRLMMNQLEEDLVSARRRSDLYESELRESRLAAEEFKRKATECQHKLLKAKDQGKPEVGEYAKLEKINAEQQLKIQELQEKLEKAAKERAERELEKLQNREDSSEGIRKKLVEAEERRHSLENKVKRLETMERRENRLKDDIQTKSQQIQQMADKILELEEKHREAQVSAQHLEVHLKQKEQHYEEKIKVLDNQIKKDLADKETLENMMQRHEEEAHEKGKILSEQKAMINAMDSKIRSLEQRIVELSEANKLAANSSLFTQRNMKAQEEMISELRQQKFYLETQAGKLEAQNRKLEEQLEKISHQDHSDKNRLLELETRLREVSLEHEEQKLELKRQLTELQLSLQERESQLTALQAARAALESQLRQAKTELEETTAEAEEEIQALTAHRDEIQRKFDALRNSCTVITDLEEQLNQLTEDNAELNNQNFYLSKQLDEASGANDEIVQLRSEVDHLRREITEREMQLTSQKQTMEALKTTCTMLEEQVMDLEALNDELLEKERQWEAWRSVLGDEKSQFECRVRELQRMLDTEKQSRARADQRITESRQVVELAVKEHKAEILALQQALKEQKLKAESLSDKLNDLEKKHAMLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKGLFSRRKEDPALPTQVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQTKEPSSSLHLEGWMKVPRNNKRGQQGWDRKYIVLEGSKVLIYDNEAREAGQRPVEEFELCLPDGDVSIHGAVGASELANTAKAEKAEADAKLLGNSLLKLEGDDRLDMNCTLPFSDQVVLVGTEEGLYALNVLKNSLTHVPGIGAVFQIYIIKDLEKLLMIAGEERALCLVDVKKVKQSLAQSHLPAQPDISPNIFEAVKGCHLFGAGKIENGLCICAAMPSKVVILRYNENLSKYCIRKEIETSEPCSCIHFTNYSILIGTNKFYEIDMKQYTLEEFLDKNDHSLAPAVFAASSNSFPVSIVQVNSAGQREEYLLCFHEFGVFVDSYGRRSRTDDLKWSRLPLAFAYREPYLFVTHFNSLEVIEIQARSSAGTPARAYLDIPNPRYLGPAISSGAIYLASSYQDKLRVICCKGNLVKESGTEHHRGPSTSRSSPNKRGPPTYNEHITKRVASSPAPPEGPSHPREPSTPHRYREGRTELRRDKSPGRPLEREKSPGRMLSTRRERSPGRLFEDSSRGRLPAGAVRTPLSQVNKGRGQSASQVFTVNTVTYYDWNKKLDNLPANWSVLRIIQLNGEIRQQVEKSVLRTDYC

SEQ ID NO: 33 - DNA, 3152 bp, Homo Sapiens
gctcgcgaatgtagcaggcgccccaagctcggtcctcaagaagccatggcggaatccaggggccgtctgtacctttggatgtgcttggctgctgcgctggcatctttcctgatgggatttatggtgggctggtttattaagcctctcaaagaaacaaccacttctgtgcgctatcatcaaagtatacggtggaaactggtatccgaaatgaaagctgaaaacatcaaatcatttcttcgttcttttacaaagcttcctcatctggcaggaacagaacaaaatttcttgcttgccaagaaaatccaaacccagtggaagaaatttggactagattcagccaagttggttcattatgatgtcctcttatcttaccccaatgagacaaatgccaactatatatcgattgtggatgaacatgaaactgagattttcaaaacatcataccttgaaccaccaccagatggctatgagaatgttacaaatattgtgccaccatataatgctttctcagcccaaggcatgccagagggagatcttgtatatgtgaactatgctcgcactgaagactttttcaaactagaaagagagatgggcatcaactgtactgggaagattgttattgcaagatatggaaaaatcttcagaggaaataaagttaaaaatgccatgttagcaggagccataggaatcatcttgtactcagatccagctgactactttgctcctgaggtacagccatatcccaaaggatggaatcttcctggaactgcagcccagagaggaaatgtgttaaatttgaatggtgctggtgacccactcactccaggctatccagcaaaagaatacactttcagacttgatgttgaagaaggagtgggaatcccccgaatacctgtacatcccattggatataatgatgcagaaatattattacgctacttgggaggaattgctccaccagataagagttggaagggagcccttaatgtgagttatagtatcggacctggctttacagggagtgattctttcaggaaggttagaatgcatgtttataacatcaataaaattacaaggatttacaatgtagttggaactatcagaggatctgtggaacctgacaggtatgttattctgggaggtcaccgggactcctgggtatttggagctattgacccaaccagtggggttgctgttttgcaagaaattgcccggagttttggaaaactgatgagtaaaggctggagacctagaagaactatcatttttgccagctgggatgcagaagaatttggacttctgggttccacagaatgggctgaggagaatgtcaaaatactccaggagagaagcattgcttatatcaactcggattcatctatagaaggcaattatactctcagagttgactgtactccccttctttaccaattagtgtataaactgacaaaagagatccccagccctgatgatgggtttgagagtaaatcactgtatgaaagctggttggaaaaagacccttcacctgaaaataaaaatttgcctagaatcaataagctgggatctggaagtgactttgaagcttattttcagagacttggaattgcttcaggcagagcccgttacactaagaataagaaaacagataagtacagcagctacccagtgtaccacacaatttatgagacatttgaattggtagagaaattttatgaccccacatttaaaaaacaactttctgtggctcaattacgaggagcactggtatatgagcttgtggattctaaaatcattccttttaatattcaagactatgcagaagctttgaaaaactatgcagcaagtatctataatctatctaagaaacatgatcaacaattaacagaccatggagtatcatttgactccttattttctgctgtgaaaaacttctcagaggctgcttcagattttcataaacgacttatacaagttgatcttaacaatcccattgcagtgagaatgatgaatgaccaactgatgctcctggaaagagcattcatcgatcctcttggtttaccaggaaagctgttctataggcacatcatatttgctccaagtagccacaacaaatatgctggagaatcatttcctggaatctatgatgctatctttgatattgaaaataaagccaactctcgtttggcctggaaagaagtaaagaaacatatttctattgcagcttttacaattcaagcagcagcaggaactctgaaagaagtattatagaaggtctcaagtggctagccattaaaggtgttgctaaaagtctgaggataaaattcacctttctgataacttatgaagccagggtgttctaaactcttttcatgtcatgttttgattataggctttggtcttttcatctgcaaagccttttttttttttgctctttaaaagttaataattatattagcaaagtgttaatctaatgaagtaaaaaactcctgtgtggcagaaagtaaaagaaaattccctaaattatagcaaggaacatgaattctcagacattgtgagtgtgggaatgtaaaatggtaaaatcacttttgaaaacagtttggcagtttcctataaagttaaacatacacttttactttaggactccagaattccacttctagttatttattcaagagaaggaaaaacaatgatcacagcaatacttgtatgcatgttcattgcaacttaaaagcgtaaaaaccccaaatgtccatccacagacgaatgtataaactgtggtatccattacacaatagactacttactactcagcaataaaaatgaagtaactttcaataaatgcaatattattggcagacattgttgaaggaaaaaagccagacaaacaactacataaaatatgtttctatttagatgaagtggcaaactaatctgtagtgttaaaaattagattagtgattgcctgggccaagtggcaggttggggaggatggctgcaaagaagtatgaggaaactttctccaatagatgagaattttccgtatcttgatctgagtggcaaattgtaaacttaaaatatatataaaatttattgaaagaaaattaagcctcaataaacgtgattataaaaaaaaaaaaaaaaagggcggccgc

SEQ ID NO: 34 - DNA, 2223 bp, Homo Sapiens
  CDS (1)...(2223)
atggcggaatccaggggccgtctgtacctttggatgtgcttggctgctgcgctggcatctttcctgatgggatttatggtgggctggtttattaagcctctcaaagaaacaaccacttctgtgcgctatcatcaaagtatacggtggaaactggtatccgaaatgaaagctgaaaacatcaaatcatttcttcgttcttttacaaagcttcctcatctggcaggaacagaacaaaatttcttgcttgccaagaaaatccaaacccagtggaagaaatttggactagattcagccaagttggttcattatgatgtcctcttatcttaccccaatgagacaaatgccaactatatatcgattgtggatgaacatgaaactgagattttcaaaacatcataccttgaaccaccaccagatggctatgagaatgttacaaatattgtgccaccatataatgctttctcagcccaaggcatgccagagggagatcttgtatatgtgaactatgctcgcactgaagactttttcaaactagaaagagagatgggcatcaactgtactgggaagattgttattgcaagatatggaaaaatcttcagaggaaataaagttaaaaatgccatgttagcaggagccataggaatcatcttgtactcagatccagctgactactttgctcctgaggtacagccatatcccaaaggatggaatcttcctggaactgcagcccagagaggaaatgtgttaaatttgaatggtgctggtgacccactcactccaggctatccagcaaaagaatacactttcagacttgatgttgaagaaggagtgggaatcccccgaatacctgtacatcccattggatataatgatgcagaaatattattacgctacttgggaggaattgctccaccagataagagttggaagggagcccttaatgtgagttatagtatcggacctggctttacagggagtgattctttcaggaaggttagaatgcatgtttataacatcaataaaattacaaggatttacaatgtagttggaactatcagaggatctgtggaacctgacaggtatgttattctgggaggtcaccgggactcctgggtatttggagctattgacccaaccagtggggttgctgttttgcaagaaattgcccggagttttggaaaactgatgagtaaaggctggagacctagaagaactatcatttttgccagctgggatgcagaagaatttggacttctgggttccacagaatgggctgaggagaatgtcaaaatactccaggagagaagcattgcttatatcaactcggattcatctatagaaggcaattatactctcagagttgactgtactccccttctttaccaattagtgtataaactgacaaaagagatccccagccctgatgatgggtttgagagtaaatcactgtatgaaagctggttggaaaaagacccttcacctgaaaataaaaatttgcctagaatcaataagctgggatctggaagtgactttgaagcttattttcagagacttggaattgcttcaggcagagcccgttacactaagaataagaaaacagataagtacagcagctacccagtgtaccacacaatttatgagacatttgaattggtagagaaattttatgaccccacatttaaaaaacaactttctgtggctcaattacgaggagcactggtatatgagcttgtggattctaaaatcattccttttaatattcaagactatgcagaagctttgaaaaactatgcagcaagtatctataatctatctaagaaacatgatcaacaattaacagaccatggagtatcatttgactccttattttctgctgtgaaaaacttctcagaggctgcttcagattttcataaacgacttatacaagttgatcttaacaatcccattgcagtgagaatgatgaatgaccaactgatgctcctggaaagagcattcatcgatcctcttggtttaccaggaaagctgttctataggcacatcatatttgctccaagtagccacaacaaatatgctggagaatcatttcctggaatctatgatgctatctttgatattgaaaataaagccaactctcgtttggcctggaaagaagtaaagaaacatatttctattgcagcttttacaattcaagcagcagcaggaactctgaaagaagtattatag

SEQ ID NO: 35 - protein, 740 aa, Homo Sapiens
MAESRGRLYLWMCLAAALASFLMGFMVGWFIKPLKETTTSVRYHQSIRWKLVSEMKAENIKSFLRSFTKLPHLAGTEQNFLLAKKIQTQWKKFGLDSAKLVHYDVLLSYPNETNANYISIVDEHETEIFKTSYLEPPPDGYENVTNIVPPYNAFSAQGMPEGDLVYVNYARTEDFFKLEREMGINCTGKIVIARYGKIFRGNKVKNAMLAGAIGIILYSDPADYFAPEVQPYPKGWNLPGTAAQRGNVLNLNGAGDPLTPGYPAKEYTFRLDVEEGVGIPRIPVHPIGYNDAEILLRYLGGIAPPDKSWKGALNVSYSIGPGFTGSDSFRKVRMHVYNINKITRIYNVVGTIRGSVEPDRYVILGGHRDSWVFGAIDPTSGVAVLQEIARSFGKLMSKGWRPRRTIIFASWDAEEFGLLGSTEWAEENVKILQERSIAYINSDSSIEGNYTLRVDCTPLLYQLVYKLTKEIPSPDDGFESKSLYESWLEKDPSPENKNLPRINKLGSGSDFEAYFQRLGIASGRARYTKNKKTDKYSSYPVYHTIYETFELVEKFYDPTFKKQLSVAQLRGALVYELVDSKIIPFNIQDYAEALKNYAASIYNLSKKHDQQLTDHGVSFDSLFSAVKNFSEAASDFHKRLIQVDLNNPIAVRMMNDQLMLLERAFIDPLGLPGKLFYRHIIFAPSSHNKYAGESFPGIYDAIFDIENKANSRLAWKEVKKHISIAAFTIQAAAGTLKEVL

SEQ ID NO: 36 - DNA, 3693 bp, Homo Sapiens
aactgtagtgcattttgccttctttccttcttagagtcacctctactttcctgctaccgctgcctgtgagctgaaggggctgaaccatacactcctttttctacaaccagcttgcattttttctgcccacaatgagcggggtaagatttttatttttggcaaggggtataatttgggttcactgtggctacttgaacactacactgcagctaactctatctttgtttcctttccaggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccactggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtgatagaaagctgagtctccctaaggcatgtgtgaaacatactcatagatgttatgcaaaaaaaagtctatggccaggtatgcatggaaaatgtgggaattaagcaaaatcaagcaagcctctctcctgcgggacttaacgtgctcatgggctgtgtgatctcttcagggtggggtggtctctgataggtagcattttccagcactttgcaaggaatgttttgtagctctagggtatatatccgcctggcatttcacaaaacagcctttgggaaatgctgaattaaagtgaattgttgacaaatgtaaacattttcagaaatattcatgaagcggtcacagatcacagtgtcttttggttacagcacaaaatgatggcagtggtttgaaaaactaaaacagaaaaaaaaatggaagccaacacatcactcattttaggcaaatgtttaaacatttttatctatcagaatgtttattgttgctggttataagcagcaggattggccggctagtgtttcctctcatttccctttgatacagtcaacaagcctgaccctgtaaaatggaggtggaaagacaagctcaagtgttcacaacctggaagtgcttcggaaagaaggggacaatggcagaacaggtgttggtgacaattgtcaccaattggataaagcagctcaggttgtagtgggccattaggaaactgtcggtttgctttgatttccctgggagctgttctctgtcgtgagtgtctcttgtctaaacgtccattaagctgagagtgctatgaagacaggatctagaataatcttgctcacagctgtgctctgagtgcctagcggagttccagcaaacaaaatggactcaagagagatttgattaatgaatcgtaatgaagttggggtttattgtacagtttaaaatgttagatgtttttaattttttaaataaatggaatacttttttttttttttaaagaaagcaactttactgagacaatgtagaaagaagttttgttccgtttctttaatgtggttgaagagcaatgtgtggctgaagacttttgttatgaggagctgcagattagctaggggacagctggaattatgctggcttctgataattattttaaaggggtctgaaatttgtgatggaatcagattttaacagctctcttcaatgacatagaaagttcatggaactcatgtttttaaagggctatgtaaatatatgaacattagaaaaatagcaacttgtgttacaaaaatacaaacacatgttaggaaggtactgtcatgggctaggcatggtggctcacacctgtaatcccagcattttgggaagctaagatgggtggatcacttgaggtcaggagtttgagaccagcctggccaacatggcgaaacccctctctactaaaaatacaaaaatttgccaggcgtggtggcgggtgcctgtaatcccagctacttgggaggctgaggcaagagaatcgcttgaacccaggaggcagaggttgcagtgagccgagatcgtgccattgcactccagcctgggtgacaaagcgagactccatctcaaaaaaaaaaaaaaaaaaaaaaggaaagaactgtcatgtaaacataccgacatgtttaaacctgacaatggtgttatttgaaactttatattgttcttgtaagctttaactatatctctctttaaaatgcaaaataatgtcttaagattcaaagtctgtatttttaaagcatggctttggctttgcaaaataaaaaatgtgttttgtacatgaagtaggaatcgtatttcagcttcaaggttcagattgaggggcccactgtttggagaggatggtattcaggctttctcatgtccttcaaatctgttagcgtttgactctagaaatcaaagcaaaggagtggttacccagacacttcttttggtgtgatcaatgcgctgatgtgatctatgaagatgattcatgcttgaaaactagcacagaaacatcttgcttatttgccaaagctgggagatgagcttctctgcataatttaaatgttcagataaatgaagctgacttatttaagcaataaccttttaaacattttagctaagatgtataaaaatgtttccaaaatataccacatactttatttcttcttaaatgtagtacattaggttacatcatttttcttgctgtcttgggcatcaaaacaggtgccatggtaacctgacactctcaggagacattaagatagaaggggctgttcttcagtggttcccattgattctccccatatctttttgctctcaggctctggccgtctcttcctgagccttaactgtgt

SEQ ID NO: 37 - DNA, 1221 bp, Homo Sapiens
  CDS (1)...(1221)
atgagcggggtaagatttttatttttggcaaggggtataatttgggttcactgtggctacttgaacactacactgcagctaactctatctttgtttcctttccaggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccactggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtga

SEQ ID NO: 38 - protein, 406 aa, Homo Sapiens [sequence omitted]

SEQ ID NO: 39 - DNA, 3164 bp, Homo Sapiens
atgccggtccgcaggggccacgtcgctccccaaaacacttacctggacaccatcatccgcaagttcgagggccaaagtcggaagttcctgattgccaatgctcagatggagaactgcgccatcatttactgcaacgacggcttctgcgaactcttcggctactcccgagtggaggtgatgcagcaaccctgcacctgcgacttcctcacaggccccaacacaccaagcagcgccgtgtcccgcctagcgcaggccctgctgggggctgaggagtgcaaggtggacatcctctactaccgcaaggatgcctccagcttccgctgcctggtagatgtggtgcccgtgaagaacgaggacggggctgtcatcatgttcattctcaacttcgaggacctggcccagctcctggccaagtgcagcagccgcagcttgtcccagcgcctgttgtcccagagcttcctgggctccgagggctctcatggcaggccaggcggaccagggccaggcacaggcaggggcaagtacaggaccatcagccagatcccacagttcacgctcaacttcgtggagttcaacttggagaagcaccgctccagctccaccacggagattgagatcatcgcgccccataaggtggtggagcggacacagaacgtcactgagaaggtcacccaggtcctgtccctgggcgcggatgtgctgccggagtacaagctgcaggcgccgcgcatccaccgctggaccatcctgcactacagccccttcaaggccgtgtgggactggctcatcctgctgctggtcatctacacggctgtcttcacgccctactcagccgccttcctgctcagcgaccaggacgaatcacggcgtggggcctgcagctatacctgcagtcccctcactgtggtggatctcatcgtggacatcatgttcgtcgtggacatcgtcatcaacttccgcaccacctatgtcaacaccaatgatgaggtggtcagccacccccgccgcatcgccgtccactacttcaagggctggttcctcattgacatggtggccgccatccctttcgacctcctgatcttccgcactggctccgatgagaccacaaccctgattgggctattgaagacagcgcggctgctgcggctggtgcgcgtagcacggaagctggaccgctactctgagtatggggcggctgtgctcttcttgctcatgtgcaccttcgcgctcatagcgcactggctggcctgcatctggtacgccatcggcaatgtggagcggccctacctagaacacaagatcggctggctggacagcctgggtgtgcagcttggcaagcgctacaacggcagcgacccagcctcgggcccctcggtgcaggacaagtatgtcacagccctctacttcaccttcagcagcctcaccagcgtgggcttcggcaatgtctcgcccaacaccaactccgagaaggtcttctccatctgcgtcatgctcatcggctccctgatgtacgccagcatcttcgggaacgtgtccgcgatcatccagcgcctgtactcgggcaccgcgcgctaccacacgcagatgctgcgtgtcaaggagttcatccgcttccaccagatccccaacccactgcgccagcgcctggaggagtatttccagcacgcctggtcctacaccaatggcattgacatgaacgcggtgctgaagggcttccccgagtgcctgcaggctgacatctgcctgcacctgcaccgcgcactgctgcagcactgcccagctttcagcggcgccggcaagggctgcctgcgcgcgctagccgtcaagttcaagaccacccacgcgccgcctggggacacgctggtgcacctcggcgacgtgctctccaccctctacttcatctcccgaggctccatcgagatcctgcgcgacgacgtggtcgtggccatcctaggaaagaatgacatctttggggaacccgtcagcctccatgcccagccaggcaagtccagtgcagacgtgcgggctctgacctactgcgacctgcacaagatccagcgggcagatctgctggaggtgctggacatgtacccggcctttgcggagagcttctggagtaagctggaggtcaccttcaacctgcgggacgcagccgggggtctccactcatccccccgacaggctcctggcagccaagaccaccaaggtttctttctcagtgacaaccagtcagatgcagcccctcccctgagcatctcagatgcatctggcctctggcctgagctactgcaggaaatgcccccaaggcacagcccccaaagccctcaggaagacccagattgctggcctctgaagctgggctccaggctagagcagctccaggcccagatgaacaggctggagtcccgcgtgtcctcagacctcagccgcatcttgcagctcctccagaagcccatgccccagggccacgccagctacattctggaagcccctgcctccaatgacctggccttggttcctatagcctcggagacgacgagtccagggcccaggctgccccagggctttctgcctcctgcacagaccccaagctatggagacttggatgactgtagtccaaagcacaggaactcctcccccaggatgcctcacctggctgtggcaacggacaaaactctggcaccatcctcagaacaggaacagcctgaggggctctggccacccctagcctcacctctacatcccctggaagtacaaggactcatctgtggtccctgcttctcctccctccctgaacaccttggctctgttcccaagcagctggacttccagagacatggctcagatcctggatttgcagggagttggggccactgaactccaagataaagacaccatgaggggactgaaggtgggcaaggggatttcctttagctgggcatggtggcgggcgcctgtaatcccagctactcaggaggctgaagcaagagaatcacttgaaccctaaaggcagaggttgcagtgagccgagatagtgccactgcactacagcccgggcgacagagtgagactccatctcaaaaataaaataaaaaaaaaaaaaaaaaaaaaaaaaaaaaaaaaaaaaaaaaaaaaaaaaaaaaaaaaaaaaaaaaaaaaaaaaa

SEQ ID NO: 40 - DNA, 2877 bp, Homo Sapiens
  CDS (1)...(2877)
atgccggtccgcaggggccacgtcgctccccaaaacacttacctggacaccatcatccgcaagttcgagggccaaagtcggaagttcctgattgccaatgctcagatggagaactgcgccatcatttactgcaacgacggcttctgcgaactcttcggctactcccgagtggaggtgatgcagcaaccctgcacctgcgacttcctcacaggccccaacacaccaagcagcgccgtgtcccgcctagcgcaggccctgctgggggctgaggagtgcaaggtggacatcctctactaccgcaaggatgcctccagcttccgctgcctggtagatgtggtgcccgtgaagaacgaggacggggctgtcatcatgttcattctcaacttcgaggacctggcccagctcctggccaagtgcagcagccgcagcttgtcccagcgcctgttgtcccagagcttcctgggctccgagggctctcatggcaggccaggcggaccagggccaggcacaggcaggggcaagtacaggaccatcagccagatcccacagttcacgctcaacttcgtggagttcaacttggagaagcaccgctccagctccaccacggagattgagatcatcgcgccccataaggtggtggagcggacacagaacgtcactgagaaggtcacccaggtcctgtccctgggcgcggatgtgctgccggagtacaagctgcaggcgccgcgcatccaccgctggaccatcctgcactacagccccttcaaggccgtgtgggactggctcatcctgctgctggtcatctacacggctgtcttcacgccctactcagccgccttcctgctcagcgaccaggacgaatcacggcgtggggcctgcagctatacctgcagtcccctcactgtggtggatctcatcgtggacatcatgttcgtcgtggacatcgtcatcaacttccgcaccacctatgtcaacaccaatgatgaggtggtcagccacccccgccgcatcgccgtccactacttcaagggctggttcctcattgacatggtggccgccatccctttcgacctcctgatcttccgcactggctccgatgagaccacaaccctgattgggctattgaagacagcgcggctgctgcggctggtgcgcgtagcacggaagctggaccgctactctgagtatggggcggctgtgctcttcttgctcatgtgcaccttcgcgctcatagcgcactggctggcctgcatctggtacgccatcggcaatgtggagcggccctacctagaacacaagatcggctggctggacagcctgggtgtgcagcttggcaagcgctacaacggcagcgacccagcctcgggcccctcggtgcaggacaagtatgtcacagccctctacttcaccttcagcagcctcaccagcgtgggcttcggcaatgtctcgcccaacaccaactccgagaaggtcttctccatctgcgtcatgctcatcggctccctgatgtacgccagcatcttcgggaacgtgtccgcgatcatccagcgcctgtactcgggcaccgcgcgctaccacacgcagatgctgcgtgtcaaggagttcatccgcttccaccagatccccaacccactgcgccagcgcctggaggagtatttccagcacgcctggtcctacaccaatggcattgacatgaacgcggtgctgaagggcttccccgagtgcctgcaggctgacatctgcctgcacctgcaccgcgcactgctgcagcactgcccagctttcagcggcgccggcaagggctgcctgcgcgcgctagccgtcaagttcaagaccacccacgcgccgcctggggacacgctggtgcacctcggcgacgtgctctccaccctctacttcatctcccgaggctccatcgagatcctgcgcgacgacgtggtcgtggccatcctaggaaagaatgacatctttggggaacccgtcagcctccatgcccagccaggcaagtccagtgcagacgtgcgggctctgacctactgcgacctgcacaagatccagcgggcagatctgctggaggtgctggacatgtacccggcctttgcggagagcttctggagtaagctggaggtcaccttcaacctgcgggacgcagccgggggtctccactcatccccccgacaggctcctggcagccaagaccaccaaggtttctttctcagtgacaaccagtcagatgcagcccctcccctgagcatctcagatgcatctggcctctggcctgagctactgcaggaaatgcccccaaggcacagcccccaaagccctcaggaagacccagattgctggcctctgaagctgggctccaggctagagcagctccaggcccagatgaacaggctggagtcccgcgtgtcctcagacctcagccgcatcttgcagctcctccagaagcccatgccccagggccacgccagctacattctggaagcccctgcctccaatgacctggccttggttcctatagcctcggagacgacgagtccagggcccaggctgccccagggctttctgcctcctgcacagaccccaagctatggagacttggatgactgtagtccaaagcacaggaactcctcccccaggatgcctcacctggctgtggcaacggacaaaactctggcaccatcctcagaacaggaacagcctgaggggctctggccacccctagcctcacctctacatcccctggaagtacaaggactcatctgtggtccctgcttctcctccctccctgaacaccttggctctgttcccaagcagctggacttccagagacatggctcagatcctggatttgcagggagttggggccactga

SEQ ID NO: 41 - protein, 958 aa, Homo Sapiens
MPVRRGHVAPQNTYLDTIIRKFEGQSRKFLIANAQMENCAIIYCNDGFCELFGYSRVEVMQQPCTCDFLTGPNTPSSAVSRLAQALLGAEECKVDILYYRKDASSFRCLVDVVPVKNEDGAVIMFILNFEDLAQLLAKCSSRSLSQRLLSQSFLGSEGSHGRPGGPGPGTGRGKYRTISQIPQFTLNFVEFNLEKHRSSSTTEIEIIAPHKVVERTQNVTEKVTQVLSLGADVLPEYKLQAPRIHRWTILHYSPFKAVWDWLILLLVIYTAVFTPYSAAFLLSDQDESRRGACSYTCSPLTVVDLIVDIMFVVDIVINFRTTYVNTNDEVVSHPRRIAVHYFKGWFLIDMVAAIPFDLLIFRTGSDETTTLIGLLKTARLLRLVRVARKLDRYSEYGAAVLFLLMCTFALIAHWLACIWYAIGNVERPYLEHKIGWLDSLGVQLGKRYNGSDPASGPSVQDKYVTALYFTFSSLTSVGFGNVSPNTNSEKVFSICVMLIGSLMYASIFGNVSAIIQRLYSGTARYHTQMLRVKEFIRFHQIPNPLRQRLEEYFQHAWSYTNGIDMNAVLKGFPECLQADICLHLHRALLQHCPAFSGAGKGCLRALAVKFKTTHAPPGDTLVHLGDVLSTLYFISRGSIEILRDDVVVAILGKNDIFGEPVSLHAQPGKSSADVRALTYCDLHKIQRADLLEVLDMYPAFAESFWSKLEVTFNLRDAAGGLHSSPRQAPGSQDHQGFFLSDNQSDAAPPLSISDASGLWPELLQEMPPRHSPQSPQEDPDCWPLKLGSRLEQLQAQMNRLESRVSSDLSRILQLLQKPMPQGHASYILEAPASNDLALVPIASETTSPGPRLPQGFLPPAQTPSYGDLDDCSPKHRNSSPRMPHLAVATDKTLAPSSEQEQPEGLWPPLASPLHPLEVQGLICGPCFSSLPEHLGSVPKQLDFQRHGSDPGFAGSWGH

(19) United States
(12) Patent Application Publication
(10) Pub. No.: US 2007/0179102 A1
(43) Pub. Date: Aug. 2, 2007

(54) Methods and compositions for the treatment and diagnosis of pain disorders using 9805, 2047, 46566, 57749, 577, 20739, 57145, 1465, 1587, 2146, 2207, 32838, 336 or 52908

(75) Inventors: Inmaculada Silos-Santiago, Del Mar, CA (US); Julie Beth Rosenfeld, Sharon, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/232,776

(22) Filed: Sep. 22, 2005

Related U.S. Application Data
(63) Continuation-in-part of application No. 10/282,942, filed on Oct. 29, 2002, now abandoned.
(63) Continuation-in-part of application No. 10/282,907, filed on Oct. 29, 2002, now abandoned.
(63) Continuation-in-part of application No. 11/183,624, filed on Jul. 18, 2005, now abandoned.
Application No. 11/183,624 is a continuation of application No. 10/281,866, filed on Oct. 28, 2002, now abandoned.
(63) Continuation-in-part of application No. 10/281,868, filed on Oct. 28, 2002, now abandoned.
(63) Continuation-in-part of application No. 10/289,161, filed on Nov. 6, 2002, now abandoned.
(63) Continuation-in-part of application No. 10/325,430, filed on Dec. 19, 2002, now abandoned.
(60) Provisional application No. 60/335,047, filed on Oct. 31, 2001.
(60) Provisional application No. 60/335,009, filed on Oct. 31, 2001.
(60) Provisional application No. 60/335,078, filed on Oct. 31, 2001.
(60) Provisional application No. 60/335,046, filed on Oct. 31, 2001.
(60) Provisional application No. 60/333,073, filed on Nov. 6, 2001.
(60) Provisional application No. 60/341,953, filed on Dec. 19, 2001.

(51) Int. Cl. A61K 48/00; C12Q 1/68; G01N 33/567

(52) U.S. Cl. 514/44; 435/6; 435/7.2

(57) ABSTRACT

The present invention relates to methods and compositions for the treatment and diagnosis of pain disorders. The invention further provides methods for identifying a compound capable of treating a pain disorder. In addition, the invention provides a method for treating a subject having a pain disorder, e.g., a pain disorder characterized by aberrant 9805, 2047, 46566, 57749, 577, 20739, 57145, 1465, 1587, 2146, 2207, 32838, 336 and 52908 polypeptide activity or aberrant 9805, 2047, 46566, 57749, 577, 20739, 57145, 1465, 1587, 2146, 2207, 32838, 336 and 52908 nucleic acid expression.

### RELATED APPLICATIONS

The present application is a continuation-in-part of U.S. patent application Ser. No. 10/282,942, filed Oct. 29, 2002, which claims the benefit of U.S. Provisional Application Ser. No. 60/335,047, filed Oct. 31, 2001, now abandoned. The present application is also a continuation-in-part of U.S. patent application Ser. No. 10/282,907, filed Oct. 29, 2002, which claims the benefit of U.S. Provisional Application Ser. No. 60/335,009, filed Oct. 31, 2001, now abandoned. The present application is also a continuation-in-part of U.S. patent application Ser. No. 11/183,624, filed Jul. 18, 2005, which is a continuation of U.S. patent application Ser. No. 10/281,866, filed Oct. 28, 2002, now abandoned, which claims the benefit of U.S. Provisional Application Ser. No. 60/335,078, filed Oct. 31, 2001, now abandoned. The present application is also a continuation-in-part of U.S. patent application Ser. No. 10/281,868, filed Oct. 28, 2002, which claims the benefit of U.S. Provisional Application Ser. No. 60/335,046, filed Oct. 31, 2001, now abandoned. The present application is also a continuation-in-part of U.S. patent application Ser. No. 10/289,161, filed Nov. 6, 2002, which claims the benefit of U.S. Provisional Application Ser. No. 60/333,073, filed Nov. 6, 2001, now abandoned. The present application is also a continuation-in-part of U.S. patent application Ser. No. 10/325,430, filed Dec. 19, 2002, which claims the benefit of U.S. Provisional Application Ser. No. 60/341,953, filed Dec. 19, 2001, now abandoned. The entire contents of patent applications are hereby incorporated by this reference.

### BACKGROUND OF THE INVENTION

The sensation of pain can be categorized into two types, peripheral and central pain. Peripheral pain can be classified into three broad areas, nociceptive pain, inflammatory pain and neuropathic pain. Nociceptive pain is also referred to as physiological pain and serves as a defense mechanism throughout the animal kingdom. Inflammatory pain, arising from severe wounds and/or associated with inflammatory infiltrates, can be well controlled by non-steroidal anti-inflammatory drugs (NSAID)-like drugs, steroids and opiates. However, the etiology and management of neuropathic pain is not well understood. Neuropathic pain is thought to arise from inherent defects in sensory and as a consequence in sympathetic neurons and can be secondary to trauma.

Pain is defined as “an unpleasant sensation occurring in varying degrees of severity as a consequence of injury, disease, or emotional disorder.” Pain is a sensation that all people must deal with at some point. Although the statistics on pain are unknown it is agreed upon that nearly all people experience pain at some point in their lives.

Pain in initiated when the peripheral terminals of a subgroup of sensory neurons are activated by noxious chemical, mechanical or thermal stimuli. These neurons, called nociceptors, transmit information regarding tissue damage to pain-processing centers in the spinal cord and brain (Fields, H. L. Pain, McGraw-Hill, New York, 1987).

The nervous system and peripheral tissues in mammals contain a large number of biologically active peptides and proteases that function as neurotransmitters or neuromodulators in the nervous system and play a role in human neurological diseases in general and pain in particular.

Peripheral pain is mediated by two types of primary sensory neuron classes, the Ad- and C-fibers, whose cell bodies lie within the dorsal root ganglion. Although the mechanisms of generation of neuropathic pain are poorly understood it is clear that several factors influence the perception and transmission of the painful stimulus, namely, alterations in chemical environment, ectopic generation of sensory neuron firing and sympathetic discharge. Some of the most common syndromes associated with neuropathic pain arise from destruction of small sensory fibers (or possibly the alteration in ratios of small to large fibers) as it is common in post-traumatic situations. Other etiologies of pain arise from small fiber damage due to diabetic neuropathy, drug induced damage (chemotherapy drugs), alcoholism, damage due to cancer, and a variety of hereditary small- and large-fiber neuropathies. We rationalize that targets derived from the peripheral nervous system may be of strategic benefit in that candidate compounds do not need to cross the blood-brain barrier, they can act on the initiation site of pain without inducing central side effects.

It has long been established that central mechanisms are involved in the perception and modulation of pain. Electrical stimulation of the periaqueductal gray (PAG) area produces analgesia without loss of other sensory modalities. Descending pain pathways emanating from PAG and the nucleus raphe magnus impinge on dorsal spinal cord regions where primary nociceptive afferents terminate. Also, stimulation of regions such as the paragigantocellularis nucleus in the medulla oblongata result in analgesia. Finally, opiate receptors, when stimulated by opioid alkaloids and opioid peptides, mediate analgesia and these sites are located in key “pain centers” within the brain including PAG, thalamic nuclei and cortical regions. Identification of genes in these CNS regions and the spinal thalamic tract from animal models of pain may elucidate important targets for pain modulation.

Tissue injury results in the production of inflammatory mediators, several of which sensitize primary afferent nociceptors resulting in hyperalgesic pain. It has been suggested that PGE-2, adenosine, and serotonin-induced hyperalgesia, as well as hyperalgesia induced by tissue damage, are initiated by activation of adenylyl cyclase-cAMP-PKA second messenger cascade. Prolonged hyperalgesia after a sustained exposure to hyperalgesic mediators may result from prolonged exposure to cAMP. The cAMP cascade is also known to have potent effects on neurotransmitter release in the central nervous system. Furthermore, application of cAMP analogues on neurons causes depolarization of the resting potential that results in increases in spontaneous synaptic activity, potentially through direct inhibition of potassium channels. In addition, cAMP activates the Vanilloid Receptor-1 (VR-1) receptor directly and sodium channels via protein kinase A activation. Another protein kinase that has been involved in nociceptive pathways mediating epinephrine, bradykinin, NGF, diabetic neuropathy and nerve ligation-induced hyperalgesia is protein kinase C.

Adenylate cyclase is a membrane bound enzyme that catalyzes the conversion of ADP to cyclic AMP (cAMP). cAMP is a second messenger that is responsible for, among other things, opening sodium channels in the dendrites of a neuron cell body. Sodium release results in an accumulation of positive ions outside the cell which ultimately causes depolarization of the membrane. This depolarization of the membrane, called the action potential, allows for messages to be carried though the neurons.

The area between the ends of two neurons is defined as the synapse. The axon of one neuron aligns with the dendrite of a neighboring neuron to create a synapse. As an action potential arrives at the axon of one neuron, calcium is released into the cytoplasm. The accumulation of calcium triggers the release of any of a number of neurotransmitters into the synapse. The released neurotransmitter binds to a receptor on the dentride of the next neuron cell, thus activating adenylate cyclase which, in turn, converts ADP into cAMP. When the action potential is generated in the postsynaptic junction the impulse is transferred to the next cell and enzymes called phosphodiesterases convert cAMP to ADP. This results in the closure of the sodium channel and the neuron being returned to its resting state.

2047, (also known as Kallikrein 6, Neurosin, zyme, protease M) is a secreted molecule and member of the kallikrein family of serine proteases, which are expressed in the nervous system as well as in some tumors. This protein is used as a biomarker for neurodegenerative diseases such as Alzheimer's and Parkinson's, and for ovarian carcinoma.

There are two principle categories of kallikreins, the plasma kallikreins (Enzyme Commission of the International Union of Biochemistry Committee on Nomenclature or “EC” Number 3.4.21.34), and the tissue kallikreins (EC number 3.4.21.35), also referred to as glandular or organ kallikreins. Plasma kallikrein, with a molecular weight of about 100,000 daltons, circulates in the blood in a precursor form, called kallikreinogen or prekallikrein. Prekallikrein is converted to its activated form by factor XII of the blood clotting cascade, known as the Hageman Factor. The principle function of plasma kallikrein is in the activation of the blood clotting and compliment enzyme cascades. In the clotting process, factor XII activates plasma kallikrein that activates additional prekallikrein and intrinsic blood coagulation.

Tissue kallikreins have been isolated most notably from urine and the pancreas, in addition to saliva, intestine, lung, brain, plasma, and a variety of other bodily cells and fluids. Biochemical studies have revealed that tissue kallikreins are heat-stable glycoproteins that consist of a single amino acid chain having a molecular weight of 27,000 to 40,000 daltons. Substrates of tissue kallikrein include procollagenase, kininogen, proinsulin, prorenin, BAM 22P, atrial naturietic factor, low density lipoprotein, atriopeptigen, and tissue plasminogen activator. These compounds are each processed or activated by proteolysis. Kininogen, in particular, requires kallikrein activity for processing and activation. Cleavage products of kininogens are kinins, e.g., bradikinin and kallidin.

Kinins are known to mediate inflammation, a cardinal feature of which is pain, and there is evidence for a primary neuronal role in the activation by kinins of peripherally located nociceptive receptors on C-fiber terminals that transmit and modulate pain perception. Kinins (bradikinin and kallidin) are released during dermal injury and inflammation as a result of activation of kallikreins (the proteolytic enzymes), which cleave kininogen. Numerous other functions have been attributed to kinins including increased chloride transport across plasma membranes, the activation of phospholipase A₂ and the release of interleukins, substance P, prostaglandins, and tumor necrosis factor. Primary roles for kinins have been shown to include the regulation of blood flow, blood pressure and sodium/water balance. Kinins have also been implicated in rheumatoid arthritis, allergic reactions, and vascular leakage (edema) from rhinitis.

Pain in the inflammatory process is a result of the release of algogenic or other substances that sensitize the pain receptors or P-fibers. Mechanical disruption is thought to play an important part in the development of pain, but pain receptors are not mechano-sensitive and pain and swelling are not related in a constant manner. Pain receptors are essentially chemoreceptive and found in the visceral and cutaneous areas of the body. Injury and regional ischemia, including thermal injury, chemical injury, electrical injury, local trauma, and localized or systemic infection, leads to the disintegration of circulating and fixed cells. These cells release lysosomal proteases, produce localized acidosis, stimulate the production of prostaglandins and vasoactive amines, and activate the kinin system. All these conditions favor the stimulation and sensitization of pain receptors.

Once a nociceptor is activated, a chain of events occur that transmit this sensation to the brain to be perceived as pain. An important step in this process is the generation of an action potential in a neuron. An action potential results in the accumulation of calcium ions in the axon terminal. This accumulation of calcium causes a release of neurotransmitter into the synapse and the propagation, ultimately, of the information regarding pain to the next neuron in the pathway from the nociceptor to the brain.

Calcium homeostasis in neurons is vital for proper control of impulses. When action potentials reach the terminal end of an axon, calcium enters the cell at a rate much faster than it can be removed. This causes the release of neurotransmitter into the synapse. In order to regulate this pathway it is vital that the cell have a mechanism to control the intracellular concentration of calcium ions.

The K⁺-dependent Na⁺/Ca⁺² exchangers are transporters of the plasma membrane of most cell types. This Na⁺/Ca⁺² exchanging activity is particularly important to excitable cells in general and neurons in particular. In these cells, K⁺-dependent Na⁺/Ca⁺² exchangers have a crucial role in the control of the Ca⁺² homeostasis in environments where the Na⁺ gradient and/or the membrane potential are lower than normal.

These Na⁺/Ca⁺² exchangers are known to be vital for proper signaling in multiple cell types including muscle and neuronal tissue. The majority of research has been done on the action of these exchangers in cardiac muscle and their importance in triggering heart contractions. On a cellular level the Na⁺/Ca⁺² exchanger from heart serve the same functional purpose as the exchangers in nerves.

Given the prevalence of pain disorders, and the lack of effective cures and early diagnostics, there currently exists a great need for methods and compositions which can serve as markers before the onset of symptoms and which can serve as a means for identifying therapeutics to treat and/or cure these disorders.

Molecules 9805, 2047, 46566 and 57749 of the Present Invention

### SUMMARY OF THE INVENTION

The present invention provides methods and compositions for the diagnosis and treatment of pain disorders.

The present invention is based, at least in part, on the discovery that expression of the 9805 gene (adenylate cyclase type VII protein) is up-regulated in various animal models of pain. Specifically, 9805 is up-regulated in the dorsal root ganglia of animals in which the sciatic nerve was constricted thereby inducing neuropathic pain; in dorsal root ganglia of monkeys in which Complete Freund's Adjuvant (CFA) was injected into the kneed joint, thereby inducing inflammatory pain; and in the dorsal root ganglia of animals after axotomy of the sciatic verve or the tibial nerve (see FIG. 2). The present invention is also based, at least in part, on the discovery that 9805 is expressed at very high levels in a subpopulation of small diameter neurons within the dorsal root ganglia that are known to be involved in pain perception.

The present invention is based, at least in part, on the discovery that the 2047 gene is upregulated in the glabrous skin innervated by the sciatic nerve after sciatic injury and after complete Freund's adjuvant (CFA) induced inflammatory pain (see FIG. 4D). Without intending to be limited by theory, it is believed that 2047 may act by releasing pain mediators that are thought to activate the nociceptors present in the injured area. Thus, the 2047 molecules, by participating in pain signaling mechanisms, can modulate pain elicitation and provide diagnostic targets and therapeutic agents to control pain and treat pain disorders.

The present invention is based, at least in part, on the discovery that 46566 (Na—Ca exchanger SLC8) is predominantly expressed in nervous tissues (the brain, spinal cord, and dorsal root ganglia (DRG)). The present invention is also based, at least in part, on the discovery that the 46566 gene is down-regulated in the spinal cord of animal models for pain, known as complete Freund's adjuvant (CFA) and axotomy models. In the CFA model, CFA is injected in the rodent paw or the monkey knee joint, thereby inducing an inflammatory response with the development of altered pain responses manifested as reduced threshold to noxious stimuli (hyperalgesia) and lowered thresholds to innocuous stimuli (allodynia). The axotomy model involves severing the sciatic nerve of an animal, thereby inducing neuropathic pain.

The present invention is based, at least in part, on the discovery that the 57749 gene (Na/Ca exchanger SLC24A) (Accession number AF097366)(SEQ ID NO:10) is most highly expressed in the dorsal root ganglia of animal models of pain. Specifically, 57749 is highly expressed in the dorsal root ganglia of monkeys in which Complete Freund's Adjuvant was injected into the knee joint, thereby inducing a model of inflammatory pain. The present invention is also based, at least in part, on the discovery that 57749 is highly expressed in nervous tissues, such as the brain, the spinal cord and the dorsal root ganglia.

Accordingly, the present invention provides methods for the diagnosis and treatment of pain disorders, e.g., arthritis, allodynia, atypical trigeminal neuralgia, trigeminal neuralgia, somatoform disorder, hypoesthesis, hypealgesia, neuralgia, heuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

In one aspect, the invention provides methods for identifying a compound capable of treating a pain disorder. The method includes assaying the ability of the compound to modulate 9805 nucleic acid expression or 9805 polypeptide activity. In one embodiment, the ability of the compound to modulate nucleic acid expression or 9805 polypeptide activity is determined by monitoring adenylate cyclase activity. In another embodiment, the ability of the compound to modulate nucleic acid expression or 9805 polypeptide activity is determined by detecting modulation of cellular second messengers (e.g., cAMP).

In another aspect, the invention provides methods for identifying a compound capable of treating a pain disorder, e.g., inflammatory pain, chronic pain and/or neuropathic pain. The method includes assaying the ability of the compound to modulate 2047 nucleic acid expression or 2047 polypeptide activity. In one embodiment, the ability of the compound to modulate nucleic acid expression or 2047 polypeptide activity is determined by detecting modulation of protease, e.g., serine protease, activity in a cell.

In another aspect, the invention provides methods for identifying a compound capable of treating a pain disorder, e.g., inflammatory pain, chronic pain and/or neuropathic pain. The method includes assaying the ability of the compound to modulate 46566 nucleic acid expression or 46566 polypeptide activity. In one embodiment, the ability of the compound to modulate nucleic acid expression or 46566 polypeptide activity is determined by detecting modulation of Na—Ca activity in a cell or by detecting intracellular calcium levels.

In another aspect, the invention provides methods for identifying a compound capable of treating a pain disorder. The method includes assaying the ability of the compound to modulate 57749 nucleic acid expression or 57749 polypeptide activity. In one embodiment, the ability of the compound to modulate nucleic acid expression or 57749 polypeptide activity is determined by monitoring the ability of a cell to exchange calcium. In another embodiment, the ability of the compound to modulate nucleic acid expression or 57749 polypeptide activity is determined by detecting modulation of the intracellular concentration of cellular second messengers (e.g., Ca).

In another aspect, the invention provides methods for identifying a compound capable of modulating pain. The method includes contacting a cell expressing an 9805 nucleic acid or polypeptide (e.g., a neuron) with a test compound and assaying the ability of the test compound to modulate the expression of an 9805 nucleic acid or the activity of an 9805 polypeptide.

In another aspect, the invention provides methods for identifying a compound capable of modulating pain and/or inflammation. The method includes contacting a cell expressing a 2047 nucleic acid or polypeptide, e.g., a neuron, with a test compound and assaying the ability of the test compound to modulate the expression of a 2047 nucleic acid or the activity of a 2047 polypeptide.

In another aspect, the invention provides methods for identifying a compound capable of modulating pain and/or inflammation. The method includes contacting a cell expressing a 46566 nucleic acid or polypeptide, e.g., a neuron, with a test compound and assaying the ability of the test compound to modulate the expression of a 46566 nucleic acid or the activity of a 46566 polypeptide.

In another aspect, the invention provides methods for identifying a compound capable of modulating pain. The method includes contacting a cell expressing a 57749 nucleic acid or polypeptide (e.g., a neuron) with a test compound and assaying the ability of the test compound to modulate the expression of a 57749 nucleic acid or the activity of a 57749 polypeptide.

In a further aspect, the invention features a method for modulating pain. The method includes contacting a cell (e.g., a neuron) with an effective amount of an 9805, 2047, 46566 or 57749 modulator, for example, an anti-9805, 2047, 46566 or 57749 antibody; an 9805, 2047, 46566 or 57749 polypeptide comprising the amino acid sequence of SEQ ID NO:2, 4, 7 or 10, or a fragment thereof; an 9805, 2047, 46566 or 57749 polypeptide comprising an amino acid sequence which is at least 90 percent identical to the amino acid sequence of SEQ ID NO:2, 4, 7 or 10; an isolated naturally occurring allelic variant of a polypeptide consisting of the amino acid sequence of SEQ ID NO:2, 4, 7 or 10; a small molecule, an antisense 9805, 2047, 46566 or 57749 nucleic acid molecule, a nucleic acid molecule of SEQ ID NO:1, 3, 5, 6, 8, 9 or 11; or a fragment thereof, or a ribozyme.

In yet another aspect, the invention features a method for treating a subject having a pain disorder, e.g. a pain disorder characterized by aberrant 9805, 2047, 46566 or 57749 polypeptide activity or aberrant 9805, 2047, 46566 or 57749 nucleic acid expression. The method includes administering to the subject a therapeutically effective amount of an 9805, 2047, 46566 or 57749 modulator, e.g., in a pharmaceutically acceptable formulation or by using a gene therapy vector. In one embodiment, the 9805, 2047, 46566 or 57749 modulator may be a small molecule, an anti-9805, 2047, 46566 or 57749 antibody, an 9805, 2047, 46566 or 57749 polypeptide comprising the amino acid sequence of SEQ ID NO:2, 4, 7 or 10, or a fragment thereof, an 9805, 2047, 46566 or 57749 polypeptide comprising an amino acid sequence which is at least 90 percent identical to the amino acid sequence of SEQ ID NO:2, 4, 7 or 10, an isolated naturally occurring allelic variant of a polypeptide consisting of the amino acid sequence of SEQ ID NO:2, 4, 7 or 10, an antisense 9805, 2047, 46566 or 57749 nucleic acid molecule, a nucleic acid molecule of SEQ ID NO:1, 3, 5, 6, 8, 9 or 11, or a fragment thereof, or a ribozyme.

In another aspect, the invention provides a method for modulating, e.g., increasing or decreasing, pain in a subject by administering to the subject a therapeutically effective amount of an 9805, 2047, 46566 or 57749 modulator.

Other features and advantages of the invention will be apparent from the following detailed description and claims.

### BRIEF DESCRIPTION OF THE DRAWINGS

FIG. 1 depicts the expression levels of 9805 in various tissues as determined by TaqMan analysis.

FIG. 2 depicts the expression levels of 9805 in various animal models as determined by TaqMan analysis.

FIG. 3A depicts a graph which demonstrates that human 2047 is upregulated in the spinal cord and the brain.

FIG. 3B depicts a graph which demonstrates that rat 2047 is upregulated in the spinal cord and the brain.

FIGS. 4A-D depict graphs which demonstrate that the 2047 gene is downregulated in various models for pain.

FIG. 5 depicts a hydrophobicity analysis of the 46566 polypeptide.

FIG. 6 is a hydrophobicity plot of the 57749 polypeptide.

### DETAILED DESCRIPTION

The present invention provides methods and compositions for the diagnosis and treatment of pain disorders.

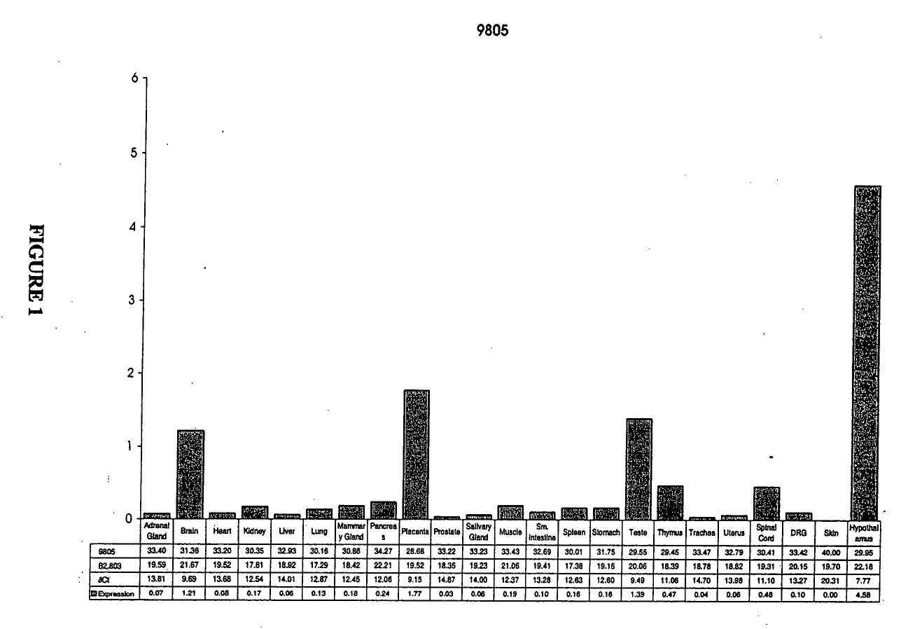

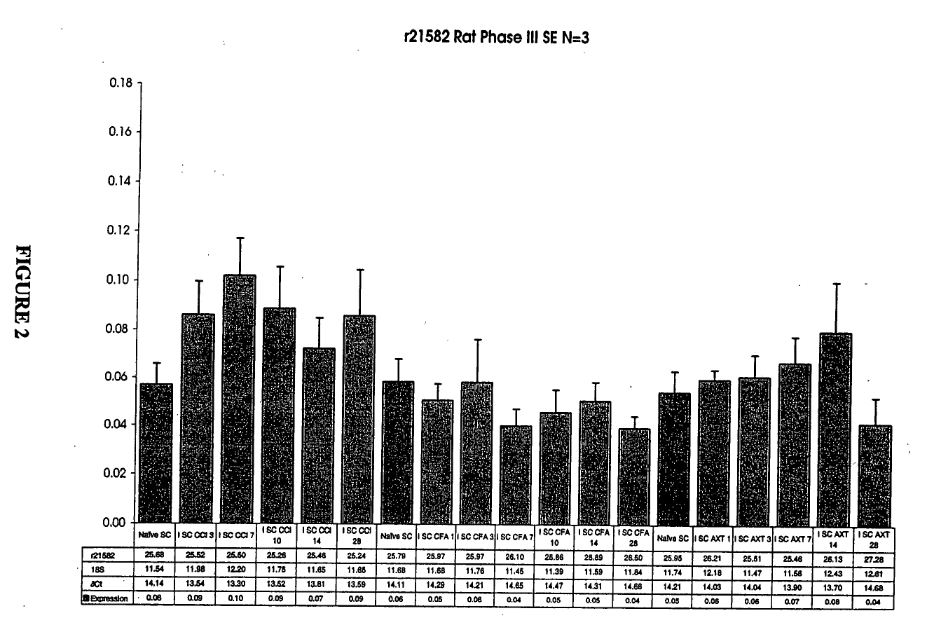

The present invention is based, at least in part, on the discovery that expression of the 9805 gene (adeny late cyclase type VII protein) is up-regulated in various animal models of pain. Specifically, 9805 is upregulated in the dorsal root ganglia of animals in which the sciatic nerve was constricted thereby inducing neuropathic pain; in dorsal root ganglia of monkeys in which Complete Freund's Adjuvant (CFA) was injected into the kneed joint, thereby inducing inflammatory pain; and in the dorsal root ganglia of animals after axotomy of the sciatic verve or the tibial nerve (see FIG. 1). The present invention is also based, at least in part, on the discovery that 9805 is expressed at very high levels in a subpopulation of small diameter neurons within the dorsal root ganglia that are known to be involved in pain perception.

Without intending to be limited by mechanism, it is believed that 9805 may be the important mediator of the cAMP transduction cascade in the dorsal root ganglia after activation of peripheral nociceptors by inflammatory mediators released after tissue injury. Thus, by participating in pain signaling mechanisms, the 9805 molecules can modulate pain elicitation and provide novel diagnostic targets and therapeutic agents to control pain and pain signaling mechanisms.

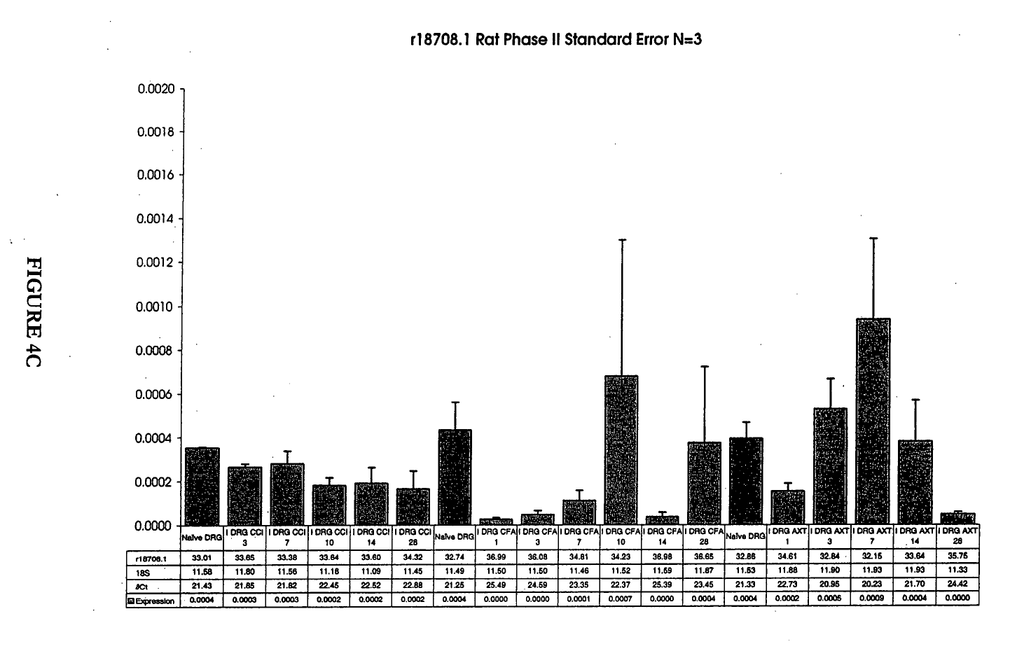

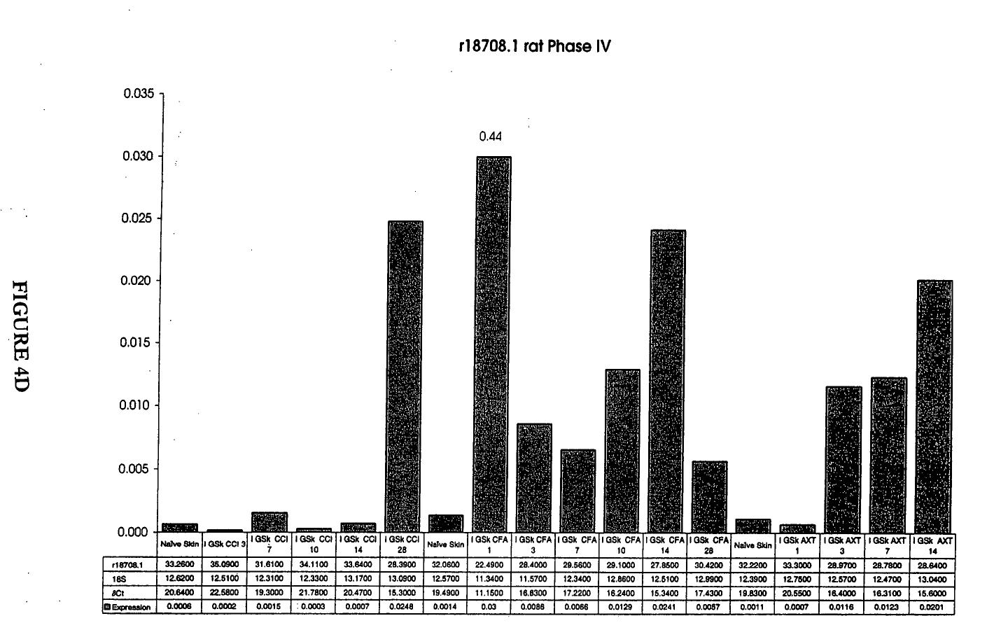

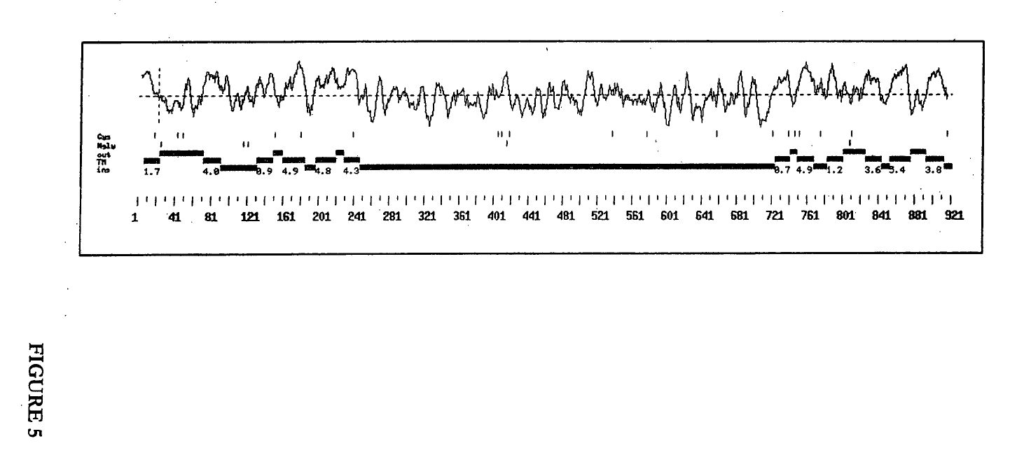

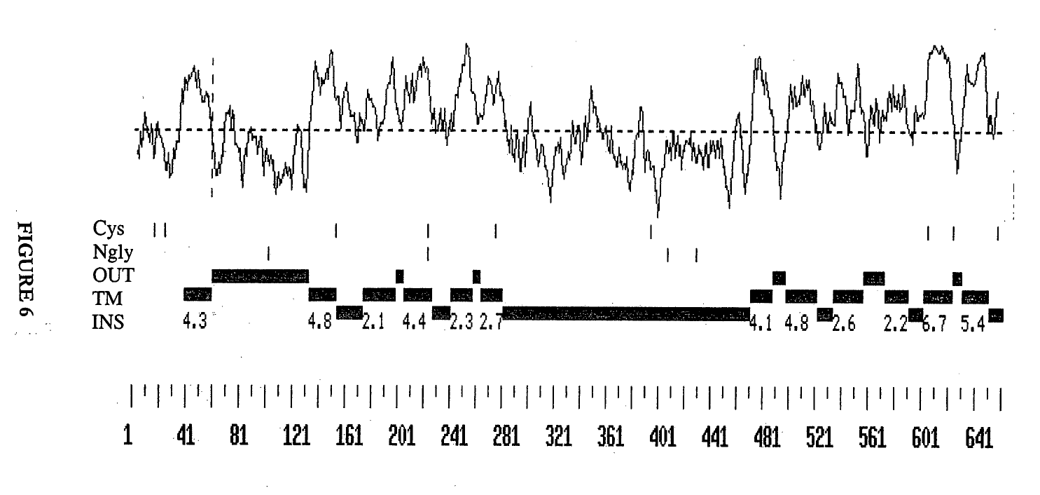

The present invention is also based, at least in part, on the discovery that the 2047 gene is upregulated in the glabrous skin innervated by the sciatic nerve after sciatic injury and after complete Freund's adjuvant (CFA) induced inflammatory pain (see FIG. 4D). Without intending to be limited by theory, it is believed that 2047 may act by releasing pain mediators that are thought to activate the nociceptors present in the injured area. Thus, the 2047 molecules, by participating in pain signaling mechanisms, can modulate pain elicitation and provide diagnostic targets and therapeutic agents to control pain and treat pain disorders.

The present invention is based, at least in part, on the discovery that 46566 is predominantly expressed in nervous tissues (the brain, spinal cord, and dorsal root ganglia (DRG)). The present invention is also based, at least in part, on the discovery that the 46566 gene is down-regulated in the spinal cord of animal models for pain, known as complete Freund's adjuvant (CFA) and axotomy models. In the CFA model, CFA is injected in the rodent paw or the monkey knee joint, thereby inducing an inflammatory response with the development of altered pain responses manifested as reduced threshold to noxious stimuli (hyperalgesia) and lowered thresholds to innocuous stimuli (allodynia). The axotomy model involves severing the sciatic nerve of an animal, thereby inducing neuropathic pain.

Without intending to be limited by theory, it is believed that the 46566 molecule may be critical for regulating the physiology of neurons involved in nociceptive pathways. Evidence demonstrating that 46566 is downregulated in the spinal cord in the inflammatory model of pain and after nerve injury suggests a significant role of this exchanger in pain signaling mechanisms. Thus, the 46566 molecules, by participating in pain signaling mechanisms, can modulate pain elicitation and provide diagnostic targets and therapeutic agents to control pain and treat pain disorders.

Multiple pathways exist in cells for transporting calcium across membranes. These include calcium channels, ATP-dependent calcium pumps, and sodium-calcium exchangers. The redundancy in function is characteristic of the importance of this function.

Among the many types of cells that actively control calcium levels, neurons rely on this ability to send impulses throughout the body. Intracellular calcium levels are responsible for both the propagation and ending of an action potential. An action potential results in the accumulation of calcium ions in the axon terminus. This accumulation of calcium causes a release of neurotransmitter into the synapse and the propagation, ultimately, of the information regarding pain to the next neuron in the pathway from the nociceptor to the brain.

Calcium homeostasis in neurons is vital for proper control of impulses. When action potentials reach the terminal end of an axon, calcium enters the cell at a rate much faster than it can be removed. This causes the release of neurotransmitter into the synapse. In order to regulate this pathway it is vital that the cell have a mechanism to control the concentration of calcium ions.

The K⁺-dependent Na⁺/Ca⁺² exchangers are transporters of the plasma membrane of most cell types. This activity is particularly important to excitable cells in general and neurons in particular. In these cells, K⁺-dependent Na⁺/Ca⁺² exchangers have a crucial role in the control of the Ca⁺² homeostasis in environments where the Na⁺ gradient and/or the membrane potential are lower than normal.

The SLC24A transporter family exchanges 4 Na⁺ for each Ca⁺² and K⁺. Furthermore, these transporters are less efficient in transporting Ca⁺² than the SLC8A family. This Na⁺/Ca⁺² exchanger may be critical for regulating the physiology of neurons involved in nociceptive pathways, especially when they are de-polarized after injury. The fact that this K⁺-dependent Na⁺/Ca⁺² exchanger is upregulated in the dorsal root ganglia of animals after the induction of inflammatory pain, as demonstrated herein, indicates an important role for this Na⁺/Ca⁺² exchanger in regulating pain signaling mechanisms in a cell. Accordingly, the NCE-SCLA24A molecules, by participating in pain signaling mechanisms, provide targets and therapeutic agents for treating, prognosing or diagnosing pain disorders.

As used herein, the term “pain signaling mechanisms” includes the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter primary afferent neurons, called polymodal nociceptors. These afferent neurons transmit the information to the central nervous system, evoking a perception of pain or discomfort and initiating appropriate protective reflexes.

As used herein, the term “pain” is art recognized and includes a bodily sensation elicited by noxious chemical, mechanical, or thermal stimuli, in a subject, e.g., a mammal such as a human. The term “pain” includes chronic pain, such as lower back pain; pain due to arthritis, e.g., osteoarthritis; joint pain, e.g., knee pain or carpal tunnel syndrome; myofascial pain, and neuropathic pain. The term “pain” further includes acute pain, such as pain associated with muscle strains and sprains; tooth pain; headaches; pain associated with surgery; or pain associated with various forms of tissue injury, e.g., inflammation, infection, and ischemia.

As used herein, the term “pain disorder” includes a disease, disorder or condition associated with or caused by pain. Examples of pain disorders include arthritis, allodynia, atypical trigeminal neuralgia, trigeminal neuralgia, somatoform disorder, hypoesthesis, hypealgesia, neuralgia, heuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

The term pain disorder, as used herein, also includes conditions or disorders which are secondary to disorders such as chronic pain and/or neuropathic pain, i.e., are influenced or caused by a disorder such as chronic pain and/or neuropathic pain. Examples of such conditions include, vasodialation, and hypotension; conditions which are behavioral, e.g., alcohol dependence (see, e.g., Hungund and Basavarajappa, (2000) Alcohol and Alcoholism 35:126-133); or conditions in which detrimental effect(s) are the result of separate disorders or injuries, e.g., spinal cord injuries.

As used interchangeably herein, “9805 activity,” “biological activity of 9805” or “functional activity of 9805, ” includes an activity exerted by the 9805 protein, polypeptide or nucleic acid molecule on an 9805 responsive cell or tissue (e.g., a neuron) or on an 9805 protein substrate, or cofactor, as determined in vivo, or in vitro, according to standard techniques. 9805 activity can be a direct activity, such as an association with an 9805-target molecule (e.g., ADP). As used herein, a “substrate” or “target molecule” or “binding partner” is a molecule with which an 9805 protein binds or interacts in nature, such that 9805-mediated function (e.g., conversion of ADP to cAMP) is achieved. An 9805 target molecule can be a non-9805 molecule (e.g., a cofactor, or a biochemical molecule involved in modulating cAMP production), or an 9805 protein or polypeptide. Examples of such target molecules include proteins in the same signaling path as the 9805 protein, e.g., proteins which may function upstream (including both stimulators and inhibitors of activity) or downstream of the 9805 protein in a pain signaling mechanism. Alternatively, an 9805 activity is an indirect activity, such as a cellular signaling activity mediated by interaction of the 9805 protein with an 9805 target molecule. The biological activities of 9805 are described herein. For example, the 9805 proteins have one or more of the following activities: (1) regulation of cAMP production in a cell, e.g., to be used as a second messenger in a signal transduction cascade; (2) modulation of a pain signaling mechanism; (3) modulation of neurotransmitter release; (4) modulation of synaptic, e.g., spontaneous synaptic, activity; (5) modulation of channel, e.g., potassium or sodium channel activity; and (6) modulation of vanilloid receptor (VR-1) receptor activity.

As used interchangeably herein, the terms “2047 activity,” “biological activity of 2047” or “functional activity of 2047,” include an activity exerted by a 2047 protein, polypeptide or nucleic acid molecule on a 2047 responsive cell or tissue or on a 2047 protein substrate, as determined in vivo, or in vitro, according to standard techniques. 2047 activity can be a direct activity, such as an association with a 2047-target molecule, e.g., kininogen. As used herein, a “substrate” or “target molecule” or “binding partner” is a molecule with which a 2047 protein binds or interacts in nature, such that 2047-mediated function, e.g., proteolytic cleavage of kininogen or modulation of a pain signaling mechanism, is achieved. A 2047 target molecule can be a non-2047 molecule (e.g., kininogen or NAD+, NADP+, or other cofactor, or a biochemical molecule involved in a pain signaling mechanism), or a 2047 protein or polypeptide. Examples of such target molecules include proteins in the same signaling path as the 2047 protein, e.g., proteins which may function upstream (including both stimulators and inhibitors of activity) or downstream of the 2047 protein in a pain signaling pathway. Alternatively, a 2047 activity is an indirect activity, such as a cellular signaling activity mediated by interaction of the 2047 protein with a 2047 target molecule. The biological activities of 2047 are described herein. For example, the 2047 proteins can have one or more of the following activities: 1) they modulate the proteolytic cleavage of kininogen; (2) they modulate the production of kinins, e.g., bradikinin and kallidin; (3) they modulate pain signaling mechanisms; (4) they modulate inflammatory responses; (5) they modulate pain perception in a subject; and (6) they modulate metabolism or catabolism of biochemical molecules (e.g., molecules involved in modulating a pain response, such as steroids that may be ligands for nuclear receptors).

As used interchangeably herein, the terms “46566 activity,” “biological activity of 46566” or “functional activity of 46566,” include an activity exerted by a 46566 protein, polypeptide or nucleic acid molecule on a 46566 responsive cell or tissue or on a 46566 protein substrate, as determined in vivo, or in vitro, according to standard techniques. 46566 activity can be a direct activity, such as an association with a 46566-target molecule. As used herein, a “substrate” or “target molecule” or “binding partner” is a molecule with which a 46566 protein binds or interacts in nature, such that 46566-mediated function, e.g., modulation of a pain signaling mechanism, is achieved. A 46566 target molecule can be a non-46566 molecule (e.g., NAD+, NADP+, or other cofactor, or a biochemical molecule involved in a pain signaling mechanism), or a 46566 protein or polypeptide. Examples of such target molecules include proteins in the same signaling path as the 46566 protein, e.g., proteins which may function upstream (including both stimulators and inhibitors of activity) or downstream of the 46566 protein in a pain signaling pathway. Alternatively, a 46566 activity is an indirect activity, such as a cellular signaling activity mediated by interaction of the 46566 protein with a 46566 target molecule. The biological activities of 46566 are described herein. For example, the 46566 proteins have one or more of the following activities: (1) regulation of Ca²⁺ production in a cell, e.g., to be used as a second messenger in a signal transduction cascade; (2) modulation of a pain signaling mechanism; (3) modulation of neurotransmitter release; (4) modulation of synaptic, e.g., spontaneous synaptic, activity; (5) regulation of sodium exchange in a cell to be used as a second messenger in a signal transduction cascade.

As used interchangeably herein, “57749 activity,” “biological activity of NCE-SLC24A” or “functional activity of NCE-SLC24A,” includes an activity exerted by the 57749 protein, polypeptide or nucleic acid molecule on a 57749 responsive cell or tissue (e.g., a neuron) or on a 57749 protein substrate, or cofactor, as determined in vivo, or in vitro, according to standard techniques. 57749 activity can be a direct activity, such as an association with a NCE-SLC24A-target molecule. As used herein, a “substrate” or “target molecule” or “binding partner” is a molecule with which a 57749 protein binds or interacts in nature, such that NCE-SLC24A-mediated function is achieved. A 57749 target molecule can be a non-57749 molecule (e.g., a cofactor, or a biochemical molecule involved in modulating cellular calcium levels), or a 57749 protein or polypeptide. Examples of such target molecules include proteins in the same signaling path as the 57749 protein, e.g., proteins which may function upstream (including both stimulators and inhibitors of activity) or downstream of the 57749 protein in a pain signaling mechanism. Alternatively, a 57749 activity is an indirect activity, such as a cellular signaling activity mediated by interaction of the 57749 protein with a 57749 target molecule. The biological activities of 57749 are described herein. For example, the 57749 proteins have one or more of the following activities (1) regulation of Ca production in a cell, e.g., to be used as a second messenger in a signal transduction cascade; (2) modulation of a pain signaling mechanism; (3) modulation of neurotransmitter release; (4) modulation of synaptic, e.g., spontaneous synaptic, activity; (5) regulation of sodium exchange in a cell to be used as a second messenger in a signal transduction cascade.

Molecules 577, 20739, 57145, 1465, 1587, 2146, 2207, 32838, 336 and 52908 of the Present Invention

### SUMMARY OF THE INVENTION

The present invention provides methods and compositions for the diagnosis and treatment of a subject experiencing pain or suffering from a painful disorders. Preferably, the subject is a human, e.g., a patient with pain or a pain-associated disorder disclosed herein. For example, the subject can be a patient with pain elicited from tissue injury, e.g., inflammation, infection, ischemia; pain associated with musculoskeletal disorders, e.g., joint pain; tooth pain; headaches, e.g., migrane; pain associated with surgery; pain related to inflammation, e.g., irritable bowel syndrome; or chest pain. The subject can be a patient with complex regional pain syndrome (CRPS), reflex sympathetic dystrophy (RSD), causalgia, neuralgia, central pain and dysesthesia syndrome, carotidynia, neurogenic pain, refractory cervicobrachial pain syndrome, myofascial pain syndrome, craniomandibular pain dysfunction syndrome, chronic idiopathic pain syndrome, Costen's pain-dysfunction, acute chest pain syndrome, gynecologic pain syndrome, patellofemoral pain syndrome, anterior knee pain syndrome, recurrent abdominal pain in children, colic, low back pain syndrome, neuropathic pain, phantom pain from amputation, phantom tooth pain, or pain asymbolia. The subject can be a cancer patient, e.g., a patient with brain cancer, bone cancer, or prostate cancer. In other embodiments, the subject is a non-human animal, e.g., an experimental animal, e.g., an arthritic rat model of chronic pain, a chronic constriction injury (CCI) rat model of neuropathic pain, or a rat model of unilateral inflammatory pain by intraplantar injection of Freund's complete adjuvant (FCA).

“Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose of curing, healing, alleviating, relieving, altering, remedying, ameliorating, improving or affecting the disease or disorder, the symptoms of disease or disorder or the predisposition toward a disease or disorder. A therapeutic agent includes, but is not limited to, the small molecules, peptides, antibodies, ribozymes and antisense oligonucleotides described herein.

The present invention is based, at least in part, on the discovery that nucleic acid and protein molecules, (described infra), are differentially expressed in animal models of pain and in peripheral and central nervous system tissues known to be associated with pain (e.g. dorsal root ganglion (DRG)). The modulators of the molecules of the present invention, identified according to the methods of the invention can be used to modulate (e.g., inhibit, treat, or prevent) pain and painful conditions.

“Differential expression”, as used herein, includes both quantitative as well as qualitative differences in the temporal and/or tissue expression pattern of a gene. Thus, a differentially expressed gene may have its expression activated or inactivated in normal versus painful disease conditions (for example, in an experimental pain model system such as in an animal model for pain). The degree to which expression differs in normal versus treated or control versus experimental states need only be large enough to be visualized via standard characterization techniques, e.g., quantitative PCR, Northern analysis, subtractive hybridization. The expression pattern of a differentially expressed gene may be used as part of a prognostic or diagnostic, evaluation, or may be used in methods for identifying compounds useful for the treatment of pain and painful disorders. In addition, a differentially expressed gene involved in pain may represent a target gene such that modulation of the level of target gene expression or of target gene product activity may act to ameliorate a painful disease condition. Compounds that modulate target gene expression or activity of the target gene product can be used in the treatment of pain or painful conditions. Although the genes described herein may be differentially expressed with respect to pain, and/or their products may interact with gene products important to pain, the genes may also be involved in mechanisms important to additional cell processes.

Molecules of the present invention include, but are not limited to ion channels (e.g. potassium channels), transporters (e.g. amino acid transporters), receptors (e.g. G protein coupled receptors) and enzymes (e.g. kinases).

Transmembrane ion channel proteins that selectively mediate the conductance of sodium, potassium, calcium and chloride ions directly modulate the electrical activity of sensory neurons and are, thus, important in nociception. In particular, potassium channels are main players in regulating the frequency and pattern of neuronal firing. The expression and peak currents of potassium channels has been shown to be regulated after different models of inflammatory and chronic pain. Additionally, calcium ions serve important intracellular signaling roles including modulation of other ion channels and regulation of protein kinases and other enzymatic activity. As cell surface proteins with established three-dimensional structures and modes of action, the pore-forming alpha subunits of ion channels make ideal drug targets. In addition to alpha subunits, these channels may consist of beta subunits and other interacting proteins which modulate channel activity and are good targets for pharmacological manipulation of the channels. Therefore, ion channels are useful in treating pain and painful conditions.

Endogenous soluble factors mediate pain sensation by binding to specific transmembrane receptors either on the peripheral terminals of nociceptive neurons or on central neurons receiving input from these nociceptors. These soluble factors include, but are not limited to serotonine, histamine, bradykinin, tachykinins (substance P and neurokinin A), opioids, eicosanoids (leukotrienes, prostaglandins, thromboxanes), purines, excitatory amino acids and different proteins. In addition a growing body of evidence, including clinical trials in man, indicates that IL-1, TNFα, and members of the neurotrophin family are involved at several stages in the transmission of painful stimuli. Hydrogen ions (protons) may mediate pain associated with inflammation (and also acid taste) by activating vanilloid receptor calcium channels or amiloride-sensitive sodium channels. Additionally, numerous exogenous agents modulate pain by mimicking endogenous soluble factors. For instance the opiate drugs of abuse exert analgesic effects by binding to receptors for the endogenous opioids and capsaicin stimulates pain sensation by binding to vanilloid receptors. The receptors for these soluble factors are linked to several signal transduction mechanisms including tyrosine kinase activity (e.g. neurotrophin receptors), recruitment of cytoplasmic tyrosine kinases (e.g. cytokine receptors for TNFα and IL-1), ion channel opening, and G-protein coupled receptors. These cell surface receptors are ideal drug targets due to their transmembrane location, and the goal is to discover G-protein coupling receptors with known ligands or with surrogate ligands that may be important players in regulating pain mechanisms.

Intracellular kinases such as protein kinase A and protein kinase C are involved in the response to pain in sensory neurons. Similarly, enzymes such as cyclooxygenase(s) and thromboxane synthetase are know to be critical in the production of prostaglandins, leukotrienes and thromboxanes. Although these particular targets may be more important in inflammatory pain, the role of this gene family in long term or neuropathic pain is of importance.

Gene ID 577

The human 577 sequence (SEQ ID NO:12), (GI:1839269, known also as sodium dependent proline transporter) which is approximately 1908 nucleotides long including untranslated regions, contains a predicted methionine-initiated coding sequence of about 1911 nucleotides, including the termination codon (nucleotides indicated as coding of SEQ ID NO:12, SEQ ID NO:13). The coding sequence encodes a 636 amino acid protein (SEQ ID NO:14) (GI:8176779).

As assessed by TaqMan analysis, 577 is expressed in the brain and dorsal root ganglion (DRG). Therefore, it is involved in nociception and would be a potential target to discover therapeutics directed toward the treatment of pain and painful disorders.

Gene ID 20739

The human 20739 sequence (SEQ ID NO:15), (GI:3608385), known also as P21-activated kinase 3 (PAK-3)) which is approximately 1635 nucleotides long including untranslated regions, contains a predicted methionine-initiated coding sequence of about 1632 nucleotides, including the termination codon (nucleotides indicated as coding of SEQ ID NO:15, SEQ ID NO:16). The coding sequence encodes a 544 amino acid protein (SEQ ID NO:17) (GI3608386).

As assessed by TaqMan analysis, 20739 is expressed in the brain, dorsal root ganglion (DRG), superior cervical ganglion (SCG), ureter, testes, ovary and spinal cord. Additional TaqMan analyses indicated that 20739 mRNA was upregulated in two rat models of pain, CFA injection and axomomy. In situ hybridization indicated that the expression of 20739 in the DRG was restricted to neurons of all sizes. Due to the expression of 20739 in DRG, SCG and brain, along with its regulated in two animal models of pain, 20739 is a potential target to discover therapeutics directed toward the treatment of pain and painful disorders.

Gene ID 57145

The human 57145 sequence (SEQ ID NO:18), (known also as OCT-5) which is approximately 2520 nucleotides long including untranslated regions, contains a predicted methionine-initiated coding sequence of about 1644 nucleotides, including the termination codon (nucleotides indicated as coding of SEQ ID NO:18, SEQ ID NO:19). The coding sequence encodes a 548 amino acid protein (SEQ ID NO:20).

As assessed by TaqMan analysis, 57145 is expressed in the brain, dorsal root ganglion (DRG), superior cervical ganglion (SCG), ureter, testes, ovary and spinal cord. Therefore, it is involved in nociception and would be a potential target to discover therapeutics directed toward the treatment of pain and painful disorders. Gene ID 1465

The human 1465 sequence (SEQ ID NO:21), (GI:971256, known also as G protein-coupled receptor kinase GRK4) which is approximately 2113 nucleotides long including untranslated regions, contains a predicted methionine-initiated coding sequence of about 1737 nucleotides, including the termination codon (nucleotides indicated as coding of SEQ ID NO:21, SEQ ID NO:22). The coding sequence encodes a 578 amino acid protein (SEQ ID NO:23) (GI:971257).

As assessed by TaqMan® analysis, 1465 was expressed in the brain and dorsal root ganglion (DRG). Therefore, it is involved in pain disorders and would be a potential target to discover therapeutics directed toward the treatment of pain and painful disorders.

Gene ID 1587

The human 1587 sequence (SEQ ID NO:24), (GI:297101), known also as serine/threonine protein kinase PCTAORE-3) which is approximately 1242 nucleotides long including untranslated regions, contains a predicted methionine-initiated coding sequence of about 1125 nucleotides, including the termination codon (nucleotides indicated as coding of SEQ ID NO:24, SEQ ID NO:25). The coding sequence encodes a 374 amino acid protein (SEQ ID NO:26) (GI:297102).

As assessed by TaqMan® analysis, 1587 shows significant expression in brain and spinal cord samples as well as heart, DRG and kidney tissues in both human and rat TaqMan® panels. Morphine experiments demonstrated a marked decrease in 1587 at 6 hours and 24 hours in the dorsal horn of the spinal cord (DH), and at 2 and 11 days in the DRG. In animal pain model systems, 1587 displayed significant down regulation in morphine and capsaicin treated animals. ISH experiments have confirmed the expression of 1587 in brain and DRG tissue samples as well as in subpopulations of glial cells. Therefore, 1587 is involved in pain response and would be a potential target to discover therapeutics directed toward the treatment of pain and painful disorders.

Gene ID 2146

The human 2146 sequence (SEQ ID NO:27), (GI:988304) (known also as protein tyrosine kinase PYK2) which is approximately 4151 nucleotides long including untranslated regions, contains a predicted methionine-initiated coding sequence of about 3030 nucleotides, including the termination codon (nucleotides indicated as coding of SEQ ID NO:27, SEQ ID NO:28). The coding sequence encodes a 1009 amino acid protein (SEQ ID NO:29) (GI:988305).

As assessed by TaqMan® analysis, 2146 was expressed at high levels in brain, DRG, spleen and peripheral blood cells. ISH experiments conducted on monkey and rat samples have confirmed the expression of 2146 in the spinal cord and DRG. In the spinal cord 2146 is expressed only in the most superficial laminae, the region involved in nociception. In the monkey and rat DRG samples, 2146 expression is observed in a very restricted subpopulation of neurons, mainly of small and intermediate diameter. Therefore, 2146 is involved in pain disorders and would be a potential target to discover therapeutics directed toward the treatment of pain and painful disorders.

Gene ID 2207

The human 2207 sequence (SEQ ID NO:30), (known also as a protein kinase) which is approximately 6574 nucleotides long including untranslated regions, contains a predicted methionine-initiated coding sequence of about 6162 nucleotides, including the termination codon (nucleotides indicated as coding of SEQ ID NO:30, SEQ ID NO:31). The coding sequence encodes a 2053 amino acid protein (SEQ ID NO:32).

As assessed by TaqMan® analysis, 2207 mRNA was highly expressed in the brain, peripheral blood cells and DRG, and at lower levels in the spinal cord. 2207 is also upregulated in the capsaicin model of pain. ISH experiments conducted on monkey and rat samples confirmed the expression of 2207 in the spinal cord and DRG. In the brain, the highest levels of expression are in the sensory thalamus. In monkey and rat DRG samples, expression of 2207 is restricted to a subpopulation of neurons of all sizes, including neurons involved in pain processing. Therefore, 2207 is involved in pain disorders and would be a potential target to discover therapeutics directed toward the treatment of pain and painful disorders.

Gene ID 32838

The human 32838 sequence (SEQ ID NO:33), (GI:4539524) (known also as NAALADase II) which is approximately 3152 nucleotides long including untranslated regions, contains a predicted methionine-initiated coding sequence of about 2223 nucleotides, including the termination codon (nucleotides indicated as coding of SEQ ID NO:33, SEQ ID NO:34). The coding sequence encodes a 740 amino acid protein (SEQ ID NO:35(GI:4535925).

As assessed by TaqMan® analysis, 32838 mRNA was restircted to expression in the brain cortex, pituitary and hypothalamus. It was also upregulated at 6 and 24 hours in the DH of animals treated with morphine. Therefore, 32838 is involved in pain disorders and would be a potential target to discover therapeutics directed toward the treatment of pain and painful disorders.

Gene ID 336

The human 336 sequence (SEQ ID NO:36), (GI:2246432) (known also as CCR6 chemokine receptor CMKBR6) which is approximately 3693 nucleotides long including untranslated regions, contains a predicted methionine-initiated coding sequence of about 1221 nucleotides, including the termination codon (nucleotides indicated as coding of SEQ ID NO:36, SEQ ID NO:37). The coding sequence encodes a 406 amino acid protein (SEQ ID NO:38) (GI:2251211).

As assessed by TaqMan® analysis, 336 mRNA was expressed in brain cortex, indicating a role in pain perception. Therefore, 336 is a potential target to discover therapeutics useful in treating pain and painful disorders.

Gene ID 52908

The human 52908 sequence (SEQ ID NO:39), (known also as a potassium channel) which is approximately 3164 nucleotides long including untranslated regions, contains a predicted methionine-initiated coding sequence of about 2877 nucleotides, including the termination codon (nucleotides indicated as coding of SEQ ID NO:39, SEQ ID NO:40). The coding sequence encodes a 958 amino acid protein (SEQ ID NO:41).

As assessed by TaqMan® analysis, 52908 mRNA expression was restricted to brain cortex, hypothalamus, spinal cord and DRG. ISH experiments confirmed the expression of 2207 in DRG and spinal cord. In the spinal cord, 52908 is expressed in a sub-population of neurons in laminae I, II and V. Also, some expression is observed around the central canal, lamina X. High levels of 52908 expression was also observed in a sub-population of nociceptive DRG neurons. This neuronal population corresponds to that of small and intermediate diameter. Therefore, 52908 is involved in pain and would be a potential target to discover therapeutics directed toward the treatment of pain and painful disorders.

Various aspects of the invention are described in further detail in the following subsections:

I. Screening Assays—9805, 2047, 46566 and 57749:

The invention provides methods (also referred to herein as “screening assays”) for identifying modulators, i.e., candidate or test compounds or agents (e.g., peptides, peptidomimetics, small molecules, ribozymes, or 9805, 2047, 46566 or 57749 antisense molecules) which bind to 9805, 2047, 46566 or 57749 proteins, have a stimulatory or inhibitory effect on 9805, 2047, 46566 or 57749 expression or 9805, 2047, 46566 or 57749 activity, or have a stimulatory or inhibitory effect on the expression or activity of an 9805, 2047, 46566 or 57749 target molecule. Compounds identified using the assays described herein may be useful for treating pain disorders.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat No. 5,223,409), spores (Ladner USP '409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one aspect, an assay is a cell-based assay in which a cell which expresses an 9805 protein or biologically active portion thereof is contacted with a test compound and the ability of the test compound to modulate 9805 activity is determined. In a preferred embodiment, the biologically active portion of the 9805 protein includes a domain or motif which can modulate a pain signaling mechanism, e.g., an ADP binding domain. Determining the ability of the test compound to modulate 9805 activity can be accomplished by monitoring, for example, the production of one or more specific metabolites (e.g., cAMP), by measuring neurotransmitter release, or by measuring synaptic activity.

In one aspect, an assay is a cell-based assay in which a cell which expresses a 2047 protein or biologically active portion thereof is contacted with a test compound and the ability of the test compound to modulate 2047 activity is determined. In a preferred embodiment, the biologically active portion of the 2047 protein includes a domain or motif that can modulate pain and/or inflammation. Determining the ability of the test compound to modulate 2047 activity can be accomplished by monitoring, for example, modulation of serine protease activity, kinin levels, or kininogen levels. The cell, for example, can be of mammalian origin.

In one aspect of the invention, serine protease activity may be measured by taking aliquots of tissue samples, e.g., cerebrospinal fluid (“CSF”) or recombinant neurosin, and incubating with 0.1 μM Boc-Phe-Ser-Arg-MCA (Peptide Inc., Osaka, Japan) in 200 μl 0.2M Tris-HCl, pH 8.0 at 37° C. (Okui, A: et al. (2001) Neurochemistry 12:1345-1350). The fluorescence activity may be measured at 380/460 nm every hour. Furthermore, the tissue samples, e.g., CSF-neurosin and recombinant mature neurosin, are electrophoresed on a gelatin copolymerized SDS-PAGE under non-reduced conditions, following incubation at 37° C. for 20 hours in 0.1 M Tris-HCl, pH 8.0. Subsequently, the gelatin gel is stained with CBB.

In one aspect, an assay is a cell-based assay in which a cell which expresses a 46566 protein or biologically active portion thereof is contacted with a test compound and the ability of the test compound to modulate 46566 activity is determined. In a preferred embodiment, the biologically active portion of the 46566 protein includes a domain or motif that can modulate pain and/or inflammation. Determining the ability of the test compound to modulate 46566 activity can be accomplished by monitoring, for example, modulation of pain and/or inflammation. The cell, for example, can be of mammalian origin.

In one aspect, an assay is a cell-based assay in which a cell which expresses a 57749 protein or biologically active portion thereof is contacted with a test compound and the ability of the test compound to modulate 57749 activity is determined. In a preferred embodiment, the biologically active portion of the 57749 protein includes a domain or motif which can modulate a pain signaling mechanism. Determining the ability of the test compound to modulate 57749 activity can be accomplished by monitoring, for example, the exchange of either sodium or calcium.

Methods for measuring sodium and calcium exchange can be found, for example, in Wood et al. (1998) J. Neurosci. 8:3208-3220, in which dorsal root ganglion cells are tested for their ability to exchange radioactive Ca²⁺.

The ability of the test compound to modulate 9805, 2047, 46566 or 57749 binding to a substrate can also be determined. Determining the ability of the test compound to modulate 9805, 2047, 46566 or 57749 binding to a substrate can be accomplished, for example, by coupling the 9805, 2047, 46566 or 57749 substrate with a radioisotope, fluorescent, or enzymatic label such that binding of the 9805, 2047, 46566 or 57749 substrate to 9805, 2047, 46566 or 57749 can be determined by detecting the labeled 9805, 2047, 46566 or 57749 substrate in a complex. Alternatively, 9805, 2047, 46566 or 57749 could be coupled with a radioisotope or enzymatic label to monitor the ability of a test compound to modulate 9805, 2047, 46566 or 57749 binding to an 9805, 2047, 46566 or 57749 substrate in a complex. Determining the ability of the test compound to bind 9805, 2047, 46566 or 57749 can be accomplished, for example, by coupling the compound with a radioisotope or enzymatic label such that binding of the compound to 9805, 2047, 46566 or 57749 can be determined by detecting the labeled 9805, 2047, 46566 or 57749 compound in a complex. For example, 9805, 2047, 46566 or 57749 substrates can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interact with 9805, 2047, 46566 or 57749 without the labeling of any of the interactants. For example, a microphysiometer can be used to detect the interaction of a compound with 9805, 2047, 46566 or 57749 without the labeling of either the compound or the 9805, 2047, 46566 or 57749 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 9805, 2047, 46566 or 57749.

Because 9805 expression is upregulated in animal models for a pain disorder, compounds which modulate pain can be identified by the ability to modulate 9805 expression. To determine whether a test compound modulates 9805 expression, a cell which expresses 9805 (e.g., a neuron) is contacted with a test compound, and the ability of the test compound to modulate 9805 expression can be determined by measuring 9805 mRNA by, e.g., Northern Blotting, quantitative PCR (e.g., TaqMan), or in vitro transcriptional assays. To perform an in vitro transcriptional assay, the full length promoter and enhancer of 9805 can be linked to a reporter gene such as chloramphenicol acetyltransferase (CAT) or luciferase and introduced into host cells. The same host cells can then be transfected with or contacted with the test compound. The effect of the test compound can be measured by reporter gene activity and comparing it to reporter gene activity in cells which do not contain the test compound. An increase or decrease in reporter gene activity indicates a modulation of 9805 expression and is, therefore, an indicator of the ability of the test compound to modulate a pain signaling mechanism in a cell.

Because 2047 expression is downregulated in the glabrous skin innervated by the sciatic nerve after sciatic injury and after CFA-induced inflammatory pain, compounds which modulate pain and/or inflammation can be identified based on their ability to modulate 2047 expression. To determine whether a test compound modulates 2047 expression, a cell which expresses 2047 is contacted with a test compound, and the ability of the test compound to modulate 2047 expression can be determined by measuring 2047 mRNA by, e.g., Northern Blotting, quantitative PCR (e.g., TaqMan), or in vitro transcriptional assays. To perform an in vitro transcriptional assay, the full length promoter and enhancer of 2047 can be linked to a reporter gene such as chloramphenicol acetyltransferase (CAT) or luciferase and introduced into host cells. The same host cells can then be transfected with or contacted with the test compound. The effect of the test compound can be measured by reporter gene activity and comparison to reporter gene activity in cells which do not contain the test compound. An increase or decrease in reporter gene activity indicates a modulation of 2047 expression and is, therefore, an indicator of the ability of the test compound to modulate pain and/or inflammation.

Because 46566 expression is downregulated in dorsal root ganglia (DRG) after axotomy, compounds which modulate pain and/or inflammation can be identified by the ability to modulate 46566 expression. To determine whether a test compound modulates 46566 expression, a cell which expresses 46566 is contacted with a test compound, and the ability of the test compound to modulate 46566 expression can be determined by measuring 46566 mRNA by, e.g., Northern Blotting, quantitative PCR (e.g., TaqMan), or in vitro transcriptional assays. To perform an in vitro transcriptional assay, the full length promoter and enhancer of 46566 can be linked to a reporter gene such as chloramphenicol acetyltransferase (CAT) or luciferase and introduced into host cells. The same host cells can then be transfected with or contacted with the test compound. The effect of the test compound can be measured by reporter gene activity and comparison to reporter gene activity in cells which do not contain the test compound. An increase or decrease in reporter gene activity indicates a modulation of 46566 expression and is, therefore, an indicator of the ability of the test compound to modulate pain and/or inflammation.

Because 57749 expression upregulated in animal models for a pain disorder, compounds which modulate pain can be identified by the ability to modulate 57749 expression. To determine whether a test compound modulates 57749 expression, a cell which expresses 57749 (e.g., a neuron) is contacted with a test compound, and the ability of the test compound to modulate 57749 expression can be determined by measuring 57749 mRNA by, e.g., Northern Blotting, quantitative PCR (e.g., TaqMan), or in vitro transcriptional assays. To perform an in vitro transcriptional assay, the full length promoter and enhancer of 57749 can be linked to a reporter gene such as chloramphenicol acetyltransferase (CAT) or luciferase and introduced into host cells. The same host cells can then be transfected with or contacted with the test compound. The effect of the test compound can be measured by reporter gene activity and comparing it to reporter gene activity in cells which do not contain the test compound. An increase or decrease in reporter gene activity indicates a modulation of 57749 expression and is, therefore, an indicator of the ability of the test compound to modulate a pain signaling mechanism in a cell.

The ability of a test compound to modulate pain can be measured by its ability to modulate cAMP production in a cell which expresses 9805, e.g., a neuron. For example, the ability of a test compound to modulate pain can be measured by contacting a cell (e.g., a muscle cell) with the test compound and measuring the levels of cAMP in the cell as compared to a control cell that was not contacted with the test compound. Compounds that modulate pain can also be identified by performing the above-described assays in animals (e.g., mice or rats) treated to induce pain. Pain can be induced in animals by, e.g., exposing them to hot or cold temperatures or treating them with a variety of compounds that induce a pain, as described herein.

Methods for measuring cAMP production can be found at, for example, Y. Salamon (1991) Methods Enzymol. 195:22-28, L. Notley-McRobb et al., (1997) Microbiology 143 (Pt 6):1909-18, Salamon, Y, et al. (1991) Methods Enzymol. 195:3-21; and Yoshima, M. et al. (1993) J. Biol. Chem. 268:4604-4607, the contents of which are incorporated by reference.

Assays that may be used to identify compounds that modulate 2047 activity also include assays that test for the ability of a compound to modulate pain and/or inflammation. The ability of a test compound to modulate pain and/or inflammation can be measured by its ability to modulate inflammation of the tissues surrounding the site of injury.

Assays that may be used to identify compounds that modulate 46566 activity also include assays that test for the ability of a compound to modulate pain and/or inflammation. The ability of a test compound to modulate pain and/or inflammation can be measured by its ability to modulate inflammation of the tissues surrounding the site of injury.

In another aspect, the invention pertains to a combination of two or more of the assays described herein. For example, a modulating agent can be identified using a cell-based or a cell-free assay, and the ability of the agent to modulate the activity of a 57749 protein can be confirmed in vivo, e.g., in an animal model for pain using, for example, rats or mice. Assays that may be used to identify compounds that modulate 57749 activity also include assays that test for the ability of a compound to modulate pain. The ability of a test compound to modulate pain can be measured by its ability to modulate calcium exchange in a cell which expresses NCE-SLC24A, e.g., a neuron. For example, the ability of a test compound to modulate pain can be measured by contacting a cell (e.g., a neuron) with the test compound and measuring the amount of calcium in the cell as compared to a control cell that not contacted with the test compound. Compounds that modulate pain can also be identified by performing the above-described assays in animals (e.g., mice or rats) treated to induce pain. Pain can be induced in animals by, e.g., exposing them to hot or cold temperatures or treating them with a variety of compounds that induce a pain, as described herein.

In yet another embodiment, an assay of the present invention is a cell-free assay in which an 9805, 2047, 46566 or 57749 protein or biologically active portion thereof is contacted with a test compound and the ability of the test compound to bind to or to modulate (e.g., stimulate or inhibit) the activity of the 9805, 2047, 46566 or 57749 protein or biologically active portion thereof is determined. Preferred biologically active portions of the 9805, 2047, 46566 or 57749 proteins to be used in assays of the present invention include fragments which participate in interactions with non-9805, 2047, 46566 or 57749 molecules, e.g., fragments with high surface probability scores. Binding of the test compound to the 9805, 2047, 46566 or 57749 protein can be determined either directly or indirectly as described above. Determining the ability of the 9805, 2047, 46566 or 57749 protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting an 9805, 2047, 46566 or 57749 protein or biologically active portion thereof with a known compound which binds the 9805, 2047, 46566 or 57749 protein to form an assay mixture, contacting the assay mixture with a test compound, and determining the ability of the test compound to interact with the 9805, 2047, 46566 or 57749 protein, wherein determining the ability of the test compound to interact with the 9805, 2047, 46566 or 57749 protein comprises determining the ability of the 9805, 2047, 46566 or 57749 protein to preferentially bind to or modulate the activity of an 9805, 2047, 46566 or 57749 target molecule (e.g., an 9805, 2047, 46566 or 57749 substrate).

The cell-free assays of the present invention are amenable to use of both soluble and/or membrane-bound forms of isolated proteins (e.g., 9805, 2047, 46566 or 57749 proteins or biologically active portions thereof). In the case of cell-free assays in which a membrane-bound form of an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either 9805, 2047, 46566 or 57749 or an 9805, 2047, 46566 or 57749 target molecule to facilitate separation of complexed from uncomplexed forms of one or both of the proteins, as well as to accommodate automation of the assay. Binding of a test compound to an 9805, 2047, 46566 or 57749 protein, or interaction of an 9805, 2047, 46566 or 57749 protein with an 9805, 2047, 46566 or 57749 target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/9805, 2047, 46566 or 57749 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 9805, 2047, 46566 or 57749 protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of 9805, 2047, 46566 or 57749 binding or activity determined using standard techniques.

Other techniques for immobilizing proteins or cell membrane preparations on matrices can also be used in the screening assays of the invention. For example, either an 9805, 2047, 46566 or 57749 protein or an 9805, 2047, 46566 or 57749 target molecule can be immobilized utilizing conjugation of biotin and streptavidin. Biotinylated 9805, 2047, 46566 or 57749 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with 9805, 2047, 46566 or 57749 protein or target molecules but which do not interfere with binding of the 9805, 2047, 46566 or 57749 protein to its target molecule can be derivatized to the wells of the plate, and unbound target or 9805, 2047, 46566 or 57749 protein is trapped in the wells by antibody conjugation. Methods for detecting such complexes, in addition to those described above for the GST-immobilized complexes, include immunodetection of complexes using antibodies reactive with the 9805, 2047, 46566 or 57749 protein or target molecule, as well as enzyme-linked assays which rely on detecting an enzymatic activity associated with the 9805, 2047, 46566 or 57749 protein or target molecule.

In yet another aspect of the invention, the 9805, 2047, 46566 or 57749 protein or fragments thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300) to identify other proteins which bind to or interact with 9805, 2047, 46566 or 57749 (“9805, 2047, 46566 or 57749-binding proteins” or “9805, 2047, 46566 or 57749-bp) and are involved in 9805, 2047, 46566 or 57749 activity. Such 9805, 2047, 46566 or 57749-binding proteins are also likely to be involved in the propagation of signals by the 9805, 2047, 46566 or 57749 proteins or 9805, 2047, 46566 or 57749 targets as, for example, downstream elements of an 9805, 2047, 46566 or 57749-mediated signaling pathway. Alternatively, such 9805, 2047, 46566 or 57749-binding proteins are likely to be 9805, 2047, 46566 or 57749 inhibitors.

The two-hybrid system is based on the modular nature of most transcription factors, which consist of separable DNA-binding and activation domains. Briefly, the assay utilizes two different DNA constructs. In one construct, the gene that codes for an 9805, 2047, 46566 or 57749 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an 9805, 2047, 46566 or 57749-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 9805, 2047, 46566 or 57749 protein.

In another aspect, the invention pertains to a combination of two or more of the assays described herein. For example, a modulating agent can be identified using a cell-based or a cell-free assay, and the ability of the agent to modulate the activity of a 9805 protein can be confirmed in vivo, e.g., in an animal model for pain using, for example, rats or mice. For example, a modulating agent can be identified using a cell-based or a cell-free assay, and the ability of the agent to modulate the activity of a 2047 protein can be confirmed in vivo, e.g., in an animal such as an animal model for chronic pain and/or neuropathic pain. For example, a modulating agent can be identified using a cell-based or a cell-free assay, and the ability of the agent to modulate the activity of a 46566 protein can be confirmed in vivo, e.g., in an animal such as an animal model for chronic pain and/or neuropathic pain. For example, a modulating agent can be identified using a cell-based or a cell-free assay, and the ability of the agent to modulate the activity of a AC-7 protein can be confirmed in vivo, e.g., in an animal model for pain using, for example, rats or mice.

Moreover, a 2047 or 46566 modulator identified as described herein (e.g., an antisense 2047 or 46566 nucleic acid molecule, a 2047 or 46566-specific antibody, or a small molecule) can be used in an animal model to determine the efficacy, toxicity, or side effects of treatment with such a modulator. Alternatively, a 2047 or 46566 modulator identified as described herein can be used in an animal model to determine the mechanism of action of such a modulator.

The ability of a given modulating agent to modulate pain can be quantitated by using any one of the following tests: tight ligation of L6 and L7, as a model of neuropathic pain; complete Freund's adjuvant into knee joint or hind paw as a model of Long term inflammatory pain (Palecek, J. (1992) Neurophysiol 68:1951-66); nerve ligation (CCI); thermal hyperalgesia, tactile allodynia and cold allodynia (Carlton, S. M. et al. (1994) Pain 56:155-66); thermal paw withdrawal latency (Hargreaves test); von Frey mechanical withdrawal threshold; the hot-plate latency test; the tail flick test (Stone, L. S., et al. (1997) NeruroReport 8:3131-3135); the warm-water immersion tail flick assay (Stone, L. S., et al. (1997) NeruroReport 8:3131-3135); the crush injury to the sciatic nerve test (De Konig, et al. (1986) J. Neurol. Sci. 74:237-246); the cold water allodynia test (Hunter, et al. (1997) Pain 69:317-322; the paw pressure latency assay (Hakki-Onen, S., et al. (2001) Brain Research 900(2):261-7; or the radiant heat test (Yoshimura, M., (2001) Pharm. Research 44(2):105-11.

Briefly, the tail flick latency test involves projecting a beam of light to the tail of an animal. The time is measured from the onset of the tail heating and stops at the moment of the tail flick. Typically, five tail flick latency (TFL) measurements are made per rat per session with 5-10 minutes between trials.

The thermal paw withdrawal latency test, also known as the Hargreaves test, consists of directing a light beam onto the ventral surface of the rats' left hindpaw from below and measuring the time until the paw is reflexively moved away from the light.

The von Frey mechanical withdrawal threshold involves placing the rat on a screen surface and attaching a von Frey filament to a force transducer. The filament is pressed upward against the ventral right hindpaw of the animal to measure the force at the instant of paw withdrawal.

The hot-plate latency test involves placing a rat onto a heated surface and measuring the time it takes the animal to jump or to lick a hindpaw.

Animal models for pain or inflammation may also be produced by the following methods: subcutaneous injection of formalin, lambda-carrageenan, mustard oil or complete Freund's adjuvant (CFA) into the right hind paw or knee of an animal which causes inflammatory pain; chronic constriction of the sciatic nerve of an animal which induces neuropathic pain; dibutylin dichloride injection in an animal which causes chronic pancreatic inflammation; axotomy of the sciatic nerve or the tibial nerve of an animal; or chronic constriction of the spinal nerves of an animal which induces neuropathic pain.

The ability of a given modulating agent to moderate the Na—Ca exchange can be quantitated by using a calcium uptake assay. The assay is performed with adult rat dorsal root ganglion cells and tests the exchange of radioactive Ca, as described by Wood et al. (1988) J. Neurosci. 8:3208-3220, herein incorporated by reference in its entirety.

Moreover, an 9805 modulator identified as described herein (e.g., an antisense 9805 nucleic acid molecule, an 9805-specific antibody, or a small molecule) can be used in an animal model to determine the efficacy, toxicity, or side effects of treatment with such a modulator. Alternatively, an 9805 modulator identified as described herein can be used in an animal model to determine the mechanism of action of such a modulator.

Moreover, a 57749 modulator identified as described herein (e.g., an antisense 57749 nucleic acid molecule, an NCE-SLC24A-specific antibody, or a small molecule) can be used in an animal model to determine the efficacy, toxicity, or side effects of treatment with such a modulator. Alternatively, a 57749 modulator identified as described herein can be used in an animal model to determine the mechanism of action of such a modulator.

II. Screening Assays—577, 20739, 57145, 1465, 1587, 2146, 2207, 32838, 336 and 52908:

The invention provides a method (also referred to herein as a “screening assay”) for identifying modulators, i.e., candidate or test compounds or agents (e.g., peptides, peptidomimetics, small molecules (organic or inorganic) or other drugs) which bind to 577, 20739, 57145, 1465, 1587, 2146, 2207, 32838, 336 or 52908 proteins, have a stimulatory or inhibitory effect on, for example, 577, 20739, 57145, 1465, 1587, 2146, 2207, 32838, 336 or 52908 expression or 577, 20739, 57145, 1465, 1587, 2146, 2207, 32838, 336 or 52908 activity, or have a stimulatory or inhibitory effect on, for example, the expression or activity of a 577, 20739, 57145, 1465, 1587, 2146, 2207, 32838, 336 or 52908 substrate. Compounds identified using the assays described herein may be useful for treating pain and painful conditions.

These assays are designed to identify compounds that bind to a 577, 20739, 57145, 1465, 1587, 2146, 2207, 32838, 336 or 52908 protein, bind to other intracellular or extracellular proteins that interact with a 577, 20739, 57145, 1465, 1587, 2146, 2207, 32838, 336 or 52908 protein, and interfere with the interaction of the 577, 20739, 57145, 1465, 1587, 2146, 2207, 32838, 336 or 52908 protein with other intercellular or extracellular proteins. For example, in the case of the 577, 20739, 57145, 1465, 1587, 2146, 2207, 32838, 336 or 52908 protein, which is a transmembrane receptor-type protein, such techniques can identify ligands for such a receptor. A 577, 20739, 57145, 1465, 1587, 2146, 2207, 32838, 336 or 52908 protein ligand or substrate can, for example, be used to ameliorate pain and painful conditions. Such compounds may include, but are not limited to peptides, antibodies, or small organic or inorganic compounds. Such compounds may also include other cellular proteins.

Compounds identified via assays such as those described herein may be useful, for example, for treating pain and painful conditions. In instances whereby a painful condition results from an overall lower level of 577, 20739, 57145, 1465, 1587, 2146, 2207, 32838, 336 or 52908 gene expression and/or 577, 20739, 57145, 1465, 1587, 2146, 2207, 32838, 336 or 52908 protein in a cell or tissue, compounds that interact with the 577, 20739, 57145, 1465, 1587, 2146, 2207, 32838, 336 or 52908 protein may include compounds which accentuate or amplify the activity of the bound 577, 20739, 57145, 1465, 1587, 2146, 2207, 32838, 336 or 52908 protein. Such compounds would bring about an effective increase in the level of 577, 20739, 57145, 1465, 1587, 2146, 2207, 32838, 336 or 52908 protein activity, thus ameliorating symptoms.

In other instances, mutations within the 577, 20739, 57145, 1465, 1587, 2146, 2207, 32838, 336 or 52908 gene may cause aberrant types or excessive amounts of 577, 20739, 57145, 1465, 1587, 2146, 2207, 32838, 336 or 52908 proteins to be made which have a deleterious effect that leads to a pain. Similarly, physiological conditions may cause an excessive increase in 577, 20739, 57145, 1465, 1587, 2146, 2207, 32838, 336 or 52908 gene expression leading pain. In such cases, compounds that bind to a 577, 20739, 57145, 1465, 1587, 2146, 2207, 32838, 336 or 52908 protein may be identified that inhibit the activity of the 577, 20739, 57145, 1465, 1587, 2146, 2207, 32838, 336 or 52908 protein. Assays for testing the effectiveness of compounds identified by techniques such as those described in this section are discussed herein.

In one embodiment, the invention provides assays for screening candidate or test compounds which are substrates of a 577, 20739, 57145, 1465, 1587, 2146, 2207, 32838, 336 or 52908 protein or polypeptide or biologically active portion thereof. In another embodiment, the invention provides assays for screening candidate or test compounds which bind to or modulate the activity of a 577, 20739, 57145, 1465, 1587, 2146, 2207, 32838, 336 or 52908 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 577, 20739, 57145, 1465, 1587, 2146, 2207, 32838, 336 or 52908 protein or biologically active portion thereof is contacted with a test compound and the ability of the test compound to modulate 577, 20739, 57145, 1465, 1587, 2146, 2207, 32838, 336 or 52908 activity is determined. Determining the ability of the test compound to modulate 577, 20739, 57145, 1465, 1587, 2146, 2207, 32838, 336 or 52908 activity can be accomplished by monitoring, for example, intracellular calcium, IP3, cAMP, or diacylglycerol concentration, the phosphorylation profile of intracellular proteins, cell proliferation and/or migration, gene expression of, for example, cell surface adhesion molecules or genes associated with analgesia, or the activity of a 577, 20739, 57145, 1465, 1587, 2146, 2207, 32838, 336 or 52908-regulated transcription factor. The cell can be of mammalian origin, e.g., a neural cell. In one embodiment, compounds that interact with a receptor domain can be screened for their ability to function as ligands, i.e., to bind to the receptor and modulate a signal transduction pathway. Identification of ligands, and measuring the activity of the ligand-receptor complex, leads to the identification of modulators (e.g., antagonists) of this interaction. Such modulators may be useful in the treatment of pain and painful conditions.

The ability of the test compound to modulate 577, 20739, 57145, 1465, 1587, 2146, 2207, 32838, 336 or 52908 binding to a substrate or to bind to 577, 20739, 57145, 1465, 1587, 2146, 2207, 32838, 336 or 52908 can also be determined. Determining the ability of the test compound to modulate 577, 20739, 57145, 1465, 1587, 2146, 2207, 32838, 336 or 52908 binding to a substrate can be accomplished, for example, by coupling the 577, 20739, 57145, 1465, 1587, 2146, 2207, 32838, 336 or 52908 substrate with a radioisotope or enzymatic label such that binding of the 577, 20739, 57145, 1465, 1587, 2146, 2207, 32838, 336 or 52908 substrate to 577, 20739, 57145, 1465, 1587, 2146, 2207, 32838, 336 or 52908 can be determined by detecting the labeled 577, 20739, 57145, 1465, 1587, 2146, 2207, 32838, 336 or 52908 substrate in a complex. 577, 20739, 57145, 1465, 1587, 2146, 2207, 32838, 336 or 52908 could also be coupled with a radioisotope or enzymatic label to monitor the ability of a test compound to modulate 577, 20739, 57145, 1465, 1587, 2146, 2207, 32838, 336 or 52908 binding to a 577, 20739, 57145, 1465, 1587, 2146, 2207, 32838, 336 or 52908 substrate in a complex. Determining the ability of the test compound to bind 577, 20739, 57145, 1465, 1587, 2146, 2207, 32838, 336 or 52908 can be accomplished, for example, by coupling the compound with a radioisotope or enzymatic label such that binding of the compound to 577, 20739, 57145, 1465, 1587, 2146, 2207, 32838, 336 or 52908 can be determined by detecting the labeled 577, 20739, 57145, 1465, 1587, 2146, 2207, 32838, 336 or 52908 compound in a complex. For example, compounds (e.g., 577, 20739, 57145, 1465, 1587, 2146, 2207, 32838, 336 or 52908 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 577, 20739, 57145, 1465, 1587, 2146, 2207, 32838, 336 or 52908 ligand or substrate) to interact with 577, 20739, 57145, 1465, 1587, 2146, 2207, 32838, 336 or 52908 without the labeling of any of the interactants. For example, a microphysiometer can be used to detect the interaction of a compound with 577, 20739, 57145, 1465, 1587, 2146, 2207, 32838, 336 or 52908 without the labeling of either the compound or the 577, 20739, 57145, 1465, 1587, 2146, 2207, 32838, 336 or 52908 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 577, 20739, 57145, 1465, 1587, 2146, 2207, 32838, 336 or 52908.

In another embodiment, an assay is a cell-based assay comprising contacting a cell expressing a 577, 20739, 57145, 1465, 1587, 2146, 2207, 32838, 336 or 52908 target molecule (e.g., a 577, 20739, 57145, 1465, 1587, 2146, 2207, 32838, 336 or 52908 substrate) with a test compound and determining the ability of the test compound to modulate (e.g., stimulate or inhibit) the activity of the 577, 20739, 57145, 1465, 1587, 2146, 2207, 32838, 336 or 52908 target molecule. Determining the ability of the test compound to modulate the activity of a 577, 20739, 57145, 1465, 1587, 2146, 2207, 32838, 336 or 52908 target molecule can be accomplished, for example, by determining the ability of the 577, 20739, 57145, 1465, 1587, 2146, 2207, 32838, 336 or 52908 protein to bind to or interact with the 577, 20739, 57145, 1465, 1587, 2146, 2207, 32838, 336 or 52908 target molecule.

Determining the ability of the 577, 20739, 57145, 1465, 1587, 2146, 2207, 32838, 336 or 52908 protein or a biologically active fragment thereof, to bind to or interact with a 577, 20739, 57145, 1465, 1587, 2146, 2207, 32838, 336 or 52908 target molecule can be accomplished by one of the methods described above for determining direct binding. In a preferred embodiment, determining the ability of the 577, 20739, 57145, 1465, 1587, 2146, 2207, 32838, 336 or 52908 protein to bind to or interact with a 577, 20739, 57145, 1465, 1587, 2146, 2207, 32838, 336 or 52908 target molecule can be accomplished by determining the activity of the target molecule. For example, the activity of the target molecule can be determined by detecting induction of a cellular second messenger of the target (i.e., intracellular Ca²⁺, diacylglycerol, IP₃, cAMP), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., gene expression).

In yet another embodiment, an assay of the present invention is a cell-free assay in which a 577, 20739, 57145, 1465, 1587, 2146, 2207, 32838, 336 or 52908 protein or biologically active portion thereof, is contacted with a test compound and the ability of the test compound to bind to the 577, 20739, 57145, 1465, 1587, 2146, 2207, 32838, 336 or 52908 protein or biologically active portion thereof is determined. Preferred biologically active portions of the 577, 20739, 57145, 1465, 1587, 2146, 2207, 32838, 336 or 52908 proteins to be used in assays of the present invention include fragments which participate in interactions with non-577, 20739, 57145, 1465, 1587, 2146, 2207, 32838, 336 or 52908 molecules, e.g., fragments with high surface probability scores. Binding of the test compound to the 577, 20739, 57145, 1465, 1587, 2146, 2207, 32838, 336 or 52908 protein can be determined either directly or indirectly as described above. In a preferred embodiment, the assay includes contacting the 577, 20739, 57145, 1465, 1587, 2146, 2207, 32838, 336 or 52908 protein or biologically active portion thereof with a known compound which binds 577, 20739, 57145, 1465, 1587, 2146, 2207, 32838, 336 or 52908 to form an assay mixture, contacting the assay mixture with a test compound, and determining the ability of the test compound to interact with a 577, 20739, 57145, 1465, 1587, 2146, 2207, 32838, 336 or 52908 protein, wherein determining the ability of the test compound to interact with a 577, 20739, 57145, 1465, 1587, 2146, 2207, 32838, 336 or 52908 protein comprises determining the ability of the test compound to preferentially bind to 577, 20739, 57145, 1465, 1587, 2146, 2207, 32838, 336 or 52908 or biologically active portion thereof as compared to the known compound. Compounds that modulate the interaction of 577, 20739, 57145, 1465, 1587, 2146, 2207, 32838, 336 or 52908 with a known target protein may be useful in regulating the activity of a 577, 20739, 57145, 1465, 1587, 2146, 2207, 32838, 336 or 52908 protein, especially a mutant 577, 20739, 57145, 1465, 1587, 2146, 2207, 32838, 336 or 52908 protein.

In another embodiment, the assay is a cell-free assay in which a 577, 20739, 57145, 1465, 1587, 2146, 2207, 32838, 336 or 52908 protein or biologically active portion thereof is contacted with a test compound and the ability of the test compound to modulate (e.g., stimulate or inhibit) the activity of the 577, 20739, 57145, 1465, 1587, 2146, 2207, 32838, 336 or 52908 protein or biologically active portion thereof is determined. Determining the ability of the test compound to modulate the activity of a 577, 20739, 57145, 1465, 1587, 2146, 2207, 32838, 336 or 52908 protein can be accomplished, for example, by determining the ability of the 577, 20739, 57145, 1465, 1587, 2146, 2207, 32838, 336 or 52908 protein to bind to a 577, 20739, 57145, 1465, 1587, 2146, 2207, 32838, 336 or 52908 target molecule by one of the methods described above for determining direct binding. Determining the ability of the 577, 20739, 57145, 1465, 1587, 2146, 2207, 32838, 336 or 52908 protein to bind to a 577, 20739, 57145, 1465, 1587, 2146, 2207, 32838, 336 or 52908 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a 577, 20739, 57145, 1465, 1587, 2146, 2207, 32838, 336 or 52908 protein can be accomplished by determining the ability of the 577, 20739, 57145, 1465, 1587, 2146, 2207, 32838, 336 or 52908 protein to further modulate the activity of a downstream effector of a 577, 20739, 57145, 1465, 1587, 2146, 2207, 32838, 336 or 52908 target molecule. For example, the activity of the effector molecule on an appropriate target can be determined or the binding of the effector to an appropriate target can be determined as previously described.

In yet another embodiment, the cell-free assay involves contacting a 577, 20739, 57145, 1465, 1587, 2146, 2207, 32838, 336 or 52908 protein or biologically active portion thereof with a known compound which binds the 577, 20739, 57145, 1465, 1587, 2146, 2207, 32838, 336 or 52908 protein to form an assay mixture, contacting the assay mixture with a test compound, and determining the ability of the test compound to interact with the 577, 20739, 57145, 1465, 1587, 2146, 2207, 32838, 336 or 52908 protein, wherein determining the ability of the test compound to interact with the 577, 20739, 57145, 1465, 1587, 2146, 2207, 32838, 336 or 52908 protein comprises determining the ability of the 577, 20739, 57145, 1465, 1587, 2146, 2207, 32838, 336 or 52908 protein to preferentially bind to or modulate the activity of a 577, 20739, 57145, 1465, 1587, 2146, 2207, 32838, 336 or 52908 target molecule.

In more than one embodiment of the above assay methods of the present invention, it may be desirable to immobilize either 577, 20739, 57145, 1465, 1587, 2146, 2207, 32838, 336 or 52908 or its target molecule to facilitate separation of complexed from uncomplexed forms of one or both of the proteins, as well as to accommodate automation of the assay. Binding of a test compound to a 577, 20739, 57145, 1465, 1587, 2146, 2207, 32838, 336 or 52908 protein, or interaction of a 577, 20739, 57145, 1465, 1587, 2146, 2207, 32838, 336 or 52908 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/577, 20739, 57145, 1465, 1587, 2146, 2207, 32838, 336 or 52908 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 577, 20739, 57145, 1465, 1587, 2146, 2207, 32838, 336 or 52908 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 577, 20739, 57145, 1465, 1587, 2146, 2207, 32838, 336 or 52908 binding or activity determined using standard techniques.

Other techniques for immobilizing proteins on matrices can also be used in the screening assays of the invention. For example, either a 577, 20739, 57145, 1465, 1587, 2146, 2207, 32838, 336 or 52908 protein or a 577, 20739, 57145, 1465, 1587, 2146, 2207, 32838, 336 or 52908 target molecule can be immobilized utilizing conjugation of biotin and streptavidin. Biotinylated 577, 20739, 57145, 1465, 1587, 2146, 2207, 32838, 336 or 52908 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 577, 20739, 57145, 1465, 1587, 2146, 2207, 32838, 336 or 52908 protein or target molecules but which do not interfere with binding of the 577, 20739, 57145, 1465, 1587, 2146, 2207, 32838, 336 or 52908 protein to its target molecule can be derivatized to the wells of the plate, and unbound target or 577, 20739, 57145, 1465, 1587, 2146, 2207, 32838, 336 or 52908 protein trapped in the wells by antibody conjugation. Methods for detecting such complexes, in addition to those described above for the GST-immobilized complexes, include immunodetection of complexes using antibodies reactive with the 577, 20739, 57145, 1465, 1587, 2146, 2207, 32838, 336 or 52908 protein or target molecule, as well as enzyme-linked assays which rely on detecting an enzymatic activity associated with the 577, 20739, 57145, 1465, 1587, 2146, 2207, 32838, 336 or 52908 protein or target molecule.

In another embodiment, modulators of 577, 20739, 57145, 1465, 1587, 2146, 2207, 32838, 336 or 52908 expression are identified in a method wherein a cell is contacted with a candidate compound and the expression of 577, 20739, 57145, 1465, 1587; 2146, 2207, 32838, 336 or 52908 mRNA or protein in the cell is determined. The level of expression of 577, 20739, 57145, 1465, 1587, 2146, 2207, 32838, 336 or 52908 mRNA or protein in the presence of the candidate compound is compared to the level of expression of 577, 20739, 57145, 1465, 1587, 2146, 2207, 32838, 336 or 52908 mRNA or protein in the absence of the candidate compound. The candidate compound can then be identified as a modulator of 577, 20739, 57145, 1465, 1587, 2146, 2207, 32838, 336 or 52908 expression based on this comparison. For example, when expression of 577, 20739, 57145, 1465, 1587, 2146, 2207, 32838, 336 or 52908 mRNA or protein is greater (statistically significantly greater) in the presence of the candidate compound than in its absence, the candidate compound is identified as a stimulator of 577, 20739, 57145, 1465, 1587, 2146, 2207, 32838, 336 or 52908 mRNA or protein expression. Alternatively, when expression of 577, 20739, 57145, 1465, 1587, 2146, 2207, 32838, 336 or 52908 mRNA or protein is less (statistically significantly less) in the presence of the candidate compound than in its absence, the candidate compound is identified as an inhibitor of 577, 20739, 57145, 1465, 1587, 2146, 2207, 32838, 336 or 52908 mRNA or protein expression. The level of 577, 20739, 57145, 1465, 1587, 2146, 2207, 32838, 336 or 52908 MRNA or protein expression in the cells can be determined by methods described herein for detecting 577, 20739, 57145, 1465, 1587, 2146, 2207, 32838, 336 or 52908 mRNA or protein.

In yet another aspect of the invention, the 577, 20739, 57145, 1465, 1587, 2146, 2207, 32838, 336 or 52908 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 577, 20739, 57145, 1465, 1587, 2146, 2207, 32838, 336 or 52908 (“577, 20739, 57145, 1465, 1587, 2146, 2207, 32838, 336 or 52908-binding proteins” or “577, 20739, 57145, 1465, 1587, 2146, 2207, 32838, 336 or 52908-bp”) and are involved in 577, 20739, 57145, 1465, 1587, 2146, 2207, 32838, 336 or 52908 activity. Such 577, 20739, 57145, 1465, 1587, 2146, 2207, 32838, 336 or 52908-binding proteins are also likely to be involved in the propagation of signals by the 577, 20739, 57145, 1465, 1587, 2146, 2207, 32838, 336 or 52908 proteins or 577, 20739, 57145, 1465, 1587, 2146, 2207, 32838, 336 or 52908 targets as, for example, downstream elements of a 577, 20739, 57145, 1465, 1587, 2146, 2207, 32838, 336 or 52908-mediated signaling pathway. Alternatively, such 577, 20739, 57145, 1465, 1587, 2146, 2207, 32838, 336 or 52908-binding proteins are likely to be 577, 20739, 57145, 1465, 1587, 2146, 2207, 32838, 336 or 52908 inhibitors.

The two-hybrid system is based on the modular nature of most transcription factors, which consist of separable DNA-binding and activation domains. Briefly, the assay utilizes two different DNA constructs. In one construct, the gene that codes for a 577, 20739, 57145, 1465, 1587, 2146, 2207, 32838, 336 or 52908 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 577, 20739, 57145, 1465, 1587, 2146, 2207, 32838, 336 or 52908-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 577, 20739, 57145, 1465, 1587, 2146, 2207, 32838, 336 or 52908 protein.

In another aspect, the invention pertains to a combination of two or more of the assays described herein. For example, a modulating agent can be identified using a cell-based or a cell free assay, and the ability of the agent to modulate the activity of a 577, 20739, 57145, 1465, 1587, 2146, 2207, 32838, 336 or 52908 protein can be confirmed in vivo, e.g., in an animal such as an animal model for pain, as described herein.

This invention further pertains to novel agents identified by the above-described screening assays. Accordingly, it is within the scope of this invention to further use an agent identified as described herein in an appropriate animal model. For example, an agent identified as described herein (e.g., a 577, 20739, 57145, 1465, 1587, 2146, 2207, 32838, 336 or 52908 modulating agent, an antisense 577, 20739, 57145, 1465, 1587, 2146, 2207, 32838, 336 or 52908 nucleic acid molecule, a 577, 20739, 57145, 1465, 1587, 2146, 2207, 32838, 336 or 52908-specific antibody, or a 577, 20739, 57145, 1465, 1587, 2146, 2207, 32838, 336 or 52908-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to ameliorate pain. Cell-based and animal model-based assays for the identification of compounds exhibiting such an ability to ameliorate pain are described herein.

In addition, animal-based models of pain, such as those described herein, may be used to identify compounds capable of treating pain and painful conditions. Such animal models may be used as test substrates for the identification of drugs, pharmaceuticals, therapies, and interventions which may be effective in treating pain. For example, animal models may be exposed to a compound, suspected of exhibiting an ability to treat pain, at a sufficient concentration and for a time sufficient to elicit such an amelioration of pain in the exposed animals. The response of the animals to the exposure may be monitored by assessing the reversal of the symptoms of pain before and after treatment.

With regard to intervention, any treatments which reverse any aspect of pain (i.e. have an analgesic effect) should be considered as candidates for human pain therapeutic intervention. Dosages of test agents may be determined by deriving dose-response curves.

Additionally, gene expression patterns may be utilized to assess the ability of a compound to ameliorate pain. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Gene expression profiles may be generated, for example, by utilizing a differential display procedure, Northern analysis and/or RT-PCR. In one embodiment, 577, 20739, 57145, 1465, 1587, 2146, 2207, 32838, 336 or 52908 gene sequences may be used as probes and/or PCR primers for the generation and corroboration of such gene expression profiles.

Gene expression profiles may be characterized for known states, either cardiovascular disease or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a pain disease model system to more closely resemble the control system. Administration of a compound may, alternatively, cause the gene expression profile of a control system to begin to mimic pain or a painful disease state. Such a compound may, for example, be used in further characterizing the compound of interest, or may be used in the generation of additional animal models.

A. Animal-Based Systems

Animal-based model systems of pain may include, but are not limited to, non-recombinant and engineered transgenic animals.

Non-recombinant animal models for pain may include, for example, genetic models.

Additionally, animal models exhibiting pain may be engineered by using, for example, 577, 20739, 57145, 1465, 1587, 2146, 2207, 32838, 336 or 52908 gene sequences described above, in conjunction with techniques for producing transgenic animals that are well known to those of skill in the art. For example, 577, 20739, 57145, 1465, 1587, 2146, 2207, 32838, 336 or 52908 gene sequences may be introduced into, and overexpressed in, the genome of the animal of interest, or, if endogenous 577, 20739, 57145, 1465, 1587, 2146, 2207, 32838, 336 or 52908 gene sequences are present, they may either be overexpressed or, alternatively, be disrupted in order to underexpress or inactivate 577, 20739, 57145, 1465, 1587, 2146, 2207, 32838, 336 or 52908 gene expression.

The host cells of the invention can also be used to produce non-human transgenic animals. For example, in one embodiment, a host cell of the invention is a fertilized oocyte or an embryonic stem cell into which 577, 20739, 57145, 1465, 1587, 2146, 2207, 32838, 336 or 52908-coding sequences have been introduced. Such host cells can then be used to create non-human transgenic animals in which exogenous 577, 20739, 57145, 1465, 1587, 2146, 2207, 32838, 336 or 52908 sequences have been introduced into their genome or homologous recombinant animals in which endogenous 577, 20739, 57145, 1465, 1587, 2146, 2207, 32838, 336 or 52908 sequences have been altered. Such animals are useful for studying the function and/or activity of a 577, 20739, 57145, 1465, 1587, 2146, 2207, 32838, 336 or 52908 and for identifying and/or evaluating modulators of 577, 20739, 57145, 1465, 1587, 2146, 2207, 32838, 336 or 52908 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 577, 20739, 57145, 1465, 1587, 2146, 2207, 32838, 336 or 52908 gene has been altered by homologous recombination between the endogenous gene and an exogenous DNA molecule introduced into a cell of the animal, e.g., an embryonic cell of the animal, prior to development of the animal.

A transgenic animal used in the methods of the invention can be created by introducing a 577, 20739, 57145, 1465, 1587, 2146, 2207, 32838, 336 or 52908-encoding nucleic acid into the male pronuclei of a fertilized oocyte, e.g., by microinjection, retroviral infection, and allowing the oocyte to develop in a pseudopregnant female foster animal. The 577, 20739, 57145, 1465, 1587, 2146, 2207, 32838, 336 or 52908 cDNA sequence can be introduced as a transgene into the genome of a non-human animal. Alternatively, a nonhuman homologue of a human 577, 20739, 57145, 1465, 1587, 2146, 2207, 32838, 336 or 52908 gene, such as a mouse or rat 577, 20739, 57145, 1465, 1587, 2146, 2207, 32838, 336 or 52908 gene, can be used as a transgene. Alternatively, a 577, 20739, 57145, 1465, 1587, 2146, 2207, 32838, 336 or 52908 gene homologue, such as another 577, 20739, 57145, 1465, 1587, 2146, 2207, 32838, 336 or 52908 family member, can be isolated based on hybridization to the 577, 20739, 57145, 1465, 1587, 2146, 2207, 32838, 336 or 52908 cDNA sequences and used as a transgene. Intronic sequences and polyadenylation signals can also be included in the transgene to increase the efficiency of expression of the transgene. A tissue-specific regulatory sequence(s) can be operably linked to a 577, 20739, 57145, 1465, 1587, 2146, 2207, 32838, 336 or 52908 transgene to direct expression of a 577, 20739, 57145, 1465, 1587, 2146, 2207, 32838, 336 or 52908 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 577, 20739, 57145, 1465, 1587, 2146, 2207, 32838, 336 or 52908 transgene in its genome and/or expression of 577, 20739, 57145, 1465, 1587, 2146, 2207, 32838, 336 or 52908 mRNA in tissues or cells of the animals. A transgenic founder animal can then be used to breed additional animals carrying the transgene. Moreover, transgenic animals carrying a transgene encoding a 577, 20739, 57145, 1465, 1587, 2146, 2207, 32838, 336 or 52908 protein can further be bred to other transgenic animals carrying other transgenes.

To create a homologous recombinant animal, a vector is prepared which contains at least a portion of a 577, 20739, 57145, 1465, 1587, 2146, 2207, 32838, 336 or 52908 gene into which a deletion, addition or substitution has been introduced to thereby alter, e.g., functionally disrupt, the 577, 20739, 57145, 1465, 1587, 2146, 2207, 32838, 336 or 52908 gene. The 577, 20739, 57145, 1465, 1587, 2146, 2207, 32838, 336 or 52908 gene can be a human gene but more preferably, is a non-human homologue of a human 577, 20739, 57145, 1465, 1587, 2146, 2207, 32838, 336 or 52908 gene. For example, a rat 577, 20739, 57145, 1465, 1587, 2146, 2207, 32838, 336 or 52908 gene can be used to construct a homologous recombination nucleic acid molecule, e.g., a vector, suitable for altering an endogenous 577, 20739, 57145, 1465, 1587, 2146, 2207, 32838, 336 or 52908 gene in the mouse genome. In a preferred embodiment, the homologous recombination nucleic acid molecule is designed such that, upon homologous recombination, the endogenous 577, 20739, 57145, 1465, 1587, 2146, 2207, 32838, 336 or 52908 gene is functionally disrupted (i.e., no longer encodes a functional protein; also referred to as a “knock out” vector). Alternatively, the homologous recombination nucleic acid molecule can be designed such that, upon homologous recombination, the endogenous 577, 20739, 57145, 1465, 1587, 2146, 2207, 32838, 336 or 52908 gene is mutated or otherwise altered but still encodes functional protein (e.g., the upstream regulatory region can be altered to thereby alter the expression of the endogenous 577, 20739, 57145, 1465, 1587, 2146, 2207, 32838, 336 or 52908 protein). In the homologous recombination nucleic acid molecule, the altered portion of the 577, 20739, 57145, 1465, 1587, 2146, 2207, 32838, 336 or 52908 gene is flanked at its 5′ and 3′ ends by additional nucleic acid sequence of the 577, 20739, 57145, 1465, 1587, 2146, 2207, 32838, 336 or 52908 gene to allow for homologous recombination to occur between the exogenous 577, 20739, 57145, 1465, 1587, 2146, 2207, 32838, 336 or 52908 gene carried by the homologous recombination nucleic acid molecule and an endogenous 577, 20739, 57145, 1465, 1587, 2146, 2207, 32838, 336 or 52908 gene in a cell, e.g., an embryonic stem cell. The additional flanking 577, 20739, 57145, 1465, 1587, 2146, 2207, 32838, 336 or 52908 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 577, 20739, 57145, 1465, 1587, 2146, 2207, 32838, 336 or 52908 gene has homologously recombined with the endogenous 577, 20739, 57145, 1465, 1587, 2146, 2207, 32838, 336 or 52908 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals for use in the methods of the invention can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 577, 20739, 57145, 1465, 1587, 2146, 2207, 32838, 336 or 52908 transgenic animals that express 577, 20739, 57145, 1465, 1587, 2146, 2207, 32838, 336 or 52908 mRNA or a 577, 20739, 57145, 1465, 1587, 2146, 2207, 32838, 336 or 52908 peptide (detected immunocytochemically, using antibodies directed against 577, 20739, 57145, 1465, 1587, 2146, 2207, 32838, 336 or 52908 epitopes) at easily detectable levels should then be further evaluated to identify those animals which display characteristic pain.

B. Cell-Based Systems

Cells that contain and express 577, 20739, 57145, 1465, 1587, 2146, 2207, 32838, 336 or 52908 gene sequences which encode a 577, 20739, 57145, 1465, 1587, 2146, 2207, 32838, 336 or 52908 protein, and, further, exhibit cellular phenotypes associated with nociception, may be used to identify compounds that exhibit analgesic effect. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); as well as generic mammalian cell lines such as HeLa cells and COS cells, e.g., COS-7 (ATCC# CRL-1651), and neural cell lines. Further, such cells may include recombinant, transgenic cell lines. For example, the pain animal models of the invention, discussed above, may be used to generate cell lines, containing one or more cell types involved in nociception, that can be used as cell culture models for this disorder. While primary cultures derived from the pain model transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in nociception may be transfected with sequences capable of increasing or decreasing the amount of 577, 20739, 57145, 1465, 1587, 2146, 2207, 32838, 336 or 52908 gene expression within the cell. For example, 577, 20739, 57145, 1465, 1587, 2146, 2207, 32838, 336 or 52908 gene sequences may be introduced into, and overexpressed in, the genome of the cell of interest, or, if endogenous 577, 20739, 57145, 1465, 1587, 2146, 2207, 32838, 336 or 52908 gene sequences are present, they may be either overexpressed or, alternatively disrupted in order to underexpress or inactivate 577, 20739, 57145, 1465, 1587, 2146, 2207, 32838, 336 or 52908 gene expression.

In order to overexpress a 577, 20739, 57145, 1465, 1587, 2146, 2207, 32838, 336 or 52908 gene, the coding portion of the 577, 20739, 57145, 1465, 1587, 2146, 2207, 32838, 336 or 52908 gene may be ligated to a regulatory sequence which is capable of driving gene expression in the cell type of interest, e.g., an endothelial cell. Such regulatory regions will be well known to those of skill in the art, and may be utilized in the absence of undue experimentation. Recombinant methods for expressing target genes are described above.

For underexpression of an endogenous 577, 20739, 57145, 1465, 1587, 2146, 2207, 32838, 336 or 52908 gene sequence, such a sequence may be isolated and engineered such that when reintroduced into the genome of the cell type of interest, the endogenous 577, 20739, 57145, 1465, 1587, 2146, 2207, 32838, 336 or 52908 alleles will be inactivated. Preferably, the engineered 577, 20739, 57145, 1465, 1587, 2146, 2207, 32838, 336 or 52908 sequence is introduced via gene targeting such that the endogenous 577, 20739, 57145, 1465, 1587, 2146, 2207, 32838, 336 or 52908 sequence is disrupted upon integration of the engineered 577, 20739, 57145, 1465, 1587, 2146, 2207, 32838, 336 or 52908 sequence into the cell's genome. Transfection of host cells with 577, 20739, 57145, 1465, 1587, 2146, 2207, 32838, 336 or 52908 genes is discussed, above.

Cells treated with compounds or transfected with 577, 20739, 57145, 1465, 1587, 2146, 2207, 32838, 336 or 52908 genes can be examined for phenotypes associated with nociception.

Transfection of 577, 20739, 57145, 1465, 1587, 2146, 2207, 32838, 336 or 52908 nucleic acid may be accomplished by using standard techniques (described in, for example, Ausubel (1989) supra). Transfected cells should be evaluated for the presence of the recombinant 577, 20739, 57145, 1465, 1587, 2146, 2207, 32838, 336 or 52908 gene sequences, for expression and accumulation of 577, 20739, 57145, 1465, 1587, 2146, 2207, 32838, 336 or 52908 mRNA, and for the presence of recombinant 577, 20739, 57145, 1465, 1587, 2146, 2207, 32838, 336 or 52908 protein production. In instances wherein a decrease in 577, 20739, 57145, 1465, 1587, 2146, 2207, 32838, 336 or 52908 gene expression is desired, standard techniques may be used to demonstrate whether a decrease in endogenous 577, 20739, 57145, 1465, 1587, 2146, 2207, 32838, 336 or 52908 gene expression and/or in 577, 20739, 57145, 1465, 1587, 2146, 2207, 32838, 336 or 52908 protein production is achieved.

III. Predictive Medicine—9805, 2047, 46566, 57749, 577, 20739, 57145, 1465, 1587, 2146, 2207, 32838, 336 and 52908:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 9805, 2047, 46566, 57749, 577, 20739, 57145, 1465, 1587, 2146, 2207, 32838, 336 or 52908 protein and/or nucleic acid expression as well as 9805, 2047, 46566, 57749, 577, 20739, 57145, 1465, 1587, 2146, 2207, 32838, 336 or 52908 activity, in the context of a biological sample (e.g., blood, serum, cells, or tissue, e.g., neuronal tissue) to thereby determine whether an individual is afflicted with a pain disorder. The invention also provides for prognostic (or predictive) assays for determining whether an individual is at risk of developing a pain. For example, mutations in an 9805, 2047, 46566, 57749, 577, 20739, 57145, 1465, 1587, 2146, 2207, 32838, 336 or 52908 gene can be assayed for in a biological sample. Such assays can be used for prognostic or predictive purpose to thereby prophylactically treat an individual prior to the onset of a pain disorder.

Another aspect of the invention pertains to monitoring the influence of 9805, 2047, 46566, 57749, 577, 20739, 57145, 1465, 1587, 2146, 2207, 32838, 336 or 52908 modulators (e.g., anti-9805, 2047, 46566, 57749, 577, 20739, 57145, 1465, 1587, 2146, 2207, 32838, 336 or 52908 antibodies or 9805, 2047, 46566, 57749, 577, 20739, 57145, 1465, 1587, 2146, 2207, 32838, 336 or 52908 ribozymes) on the expression or activity of 9805, 2047, 46566, 57749, 577, 20739, 57145, 1465, 1587, 2146, 2207, 32838, 336 or 52908 in clinical trials.

These and other agents are described in further detail in the following sections.

A. Diagnostic Assays for Pain Disorders

To determine whether a subject is afflicted with a pain disorder, a biological sample may be obtained from a subject and the biological sample may be contacted with a compound or an agent capable of detecting an 9805, 2047, 46566, 57749, 577, 20739, 57145, 1465, 1587, 2146, 2207, 32838, 336 or 52908 protein or nucleic acid (e.g., mRNA or genomic DNA) that encodes an 9805, 2047, 46566, 57749, 577, 20739, 57145, 1465, 1587, 2146, 2207, 32838, 336 or 52908 protein, in the biological sample. A preferred agent for detecting 9805, 2047, 46566, 57749, 577, 20739, 57145, 1465, 1587, 2146, 2207, 32838, 336 or 52908 mRNA or genomic DNA is a labeled nucleic acid probe capable of hybridizing to 9805, 2047, 46566, 57749, 577, 20739, 57145, 1465, 1587, 2146, 2207, 32838, 336 or 52908 mRNA or genomic DNA. The nucleic acid probe can be, for example, the 9805, 2047, 46566, 57749, 577, 20739, 57145, 1465, 1587, 2146, 2207, 32838, 336 or 52908 nucleic acid set forth in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, or a portion thereof, such as an oligonucleotide of at least 15, 20, 25, 30, 25, 40, 45, 50, 100, 250 or 500 nucleotides in length and sufficient to specifically hybridize under stringent conditions to 9805, 2047, 46566, 57749, 577, 20739, 57145, 1465, 1587, 2146, 2207, 32838, 336 or 52908 mRNA or genomic DNA. Other suitable probes for use in the diagnostic assays of the invention are described herein.

A preferred agent for detecting 9805, 2047, 46566, 57749, 577, 20739, 57145, 1465, 1587, 2146, 2207, 32838, 336 or 52908 protein in a sample is an antibody capable of binding to 9805, 2047, 46566, 57749, 577, 20739, 57145, 1465, 1587, 2146, 2207, 32838, 336 or 52908 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of direct substances that can be coupled to an antibody or a nucleic acid probe include various enzymes, prosthetic groups, fluorescent materials, luminescent materials, bioluminescent materials, and radioactive materials. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 9805, 2047, 46566, 57749, 577, 20739, 57145, 1465, 1587, 2146, 2207, 32838, 336 or 52908 mRNA, protein, or genomic DNA in a biological sample in vitro as well as in vivo. For example, in vitro techniques for detection of 9805, 2047, 46566, 57749, 577, 20739, 57145, 1465, 1587, 2146, 2207, 32838, 336 or 52908 mRNA include Northern hybridizations and in situ hybridizations. In vitro techniques for detection of 9805, 2047, 46566, 57749, 577, 20739, 57145, 1465, 1587, 2146, 2207, 32838, 336 or 52908 protein include enzyme linked immunosorbent assays (ELISAs), Western blots, immunoprecipitations and immunofluorescence. In vitro techniques for detection of 9805, 2047, 46566, 57749, 577, 20739, 57145, 1465, 1587, 2146, 2207, 32838, 336 or 52908 genomic DNA include Southern hybridizations. Furthermore, in vivo techniques for detection of 9805, 2047, 46566, 57749, 577, 20739, 57145, 1465, 1587, 2146, 2207, 32838, 336 or 52908 protein include introducing into a subject a labeled anti-9805, 2047, 46566, 57749, 577, 20739, 57145, 1465, 1587, 2146, 2207, 32838, 336 or 52908 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 9805, 2047, 46566, 57749, 577, 20739, 57145, 1465, 1587, 2146, 2207, 32838, 336 or 52908 protein, mRNA, or genomic DNA, such that the presence of 9805, 2047, 46566, 57749, 577, 20739, 57145, 1465, 1587, 2146, 2207, 32838, 336 or 52908 protein, mRNA or genomic DNA is detected in the biological sample, and comparing the presence of 9805, 2047, 46566, 57749, 577, 20739, 57145, 1465, 1587, 2146, 2207, 32838, 336 or 52908 protein, mRNA or genomic DNA in the control sample with the presence of 9805, 2047, 46566, 57749, 577, 20739, 57145, 1465, 1587, 2146, 2207, 32838, 336 or 52908 protein, mRNA or genomic DNA in the test sample.

B. Prognostic Assays for Pain Disorders

The present invention further pertains to methods for identifying subjects having or at risk of developing a pain disorder with aberrant 9805, 2047, 46566, 57749, 577, 20739, 57145, 1465, 1587, 2146, 2207, 32838, 336 or 52908 expression or activity.

As used herein, the term “aberrant” includes an 9805, 2047, 46566, 57749, 577, 20739, 57145, 1465, 1587, 2146, 2207, 32838, 336 or 52908 expression or activity which deviates from the wild type 9805, 2047, 46566, 57749, 577, 20739, 57145, 1465, 1587, 2146, 2207, 32838, 336 or 52908 expression or activity. Aberrant expression or activity includes increased or decreased expression or activity, as well as expression or activity which does not follow the wild type developmental pattern of expression or the subcellular pattern of expression. For example, aberrant 9805, 2047, 46566, 57749, 577, 20739, 57145, 1465, 1587, 2146, 2207, 32838, 336 or 52908 expression or activity is intended to include the cases in which a mutation in the 9805, 2047, 46566, 57749, 577, 20739, 57145, 1465, 1587, 2146, 2207, 32838, 336 or 52908 gene causes the 9805, 2047, 46566, 57749, 577, 20739, 57145, 1465, 1587, 2146, 2207, 32838, 336 or 52908 gene to be under-expressed or over-expressed and situations in which such mutations result in a non-functional 9805, 2047, 46566, 57749, 577, 20739, 57145, 1465, 1587, 2146, 2207, 32838, 336 or 52908 protein or a protein which does not function in a wild-type fashion, e.g., a protein which does not interact with an 9805, 2047, 46566, 57749, 577, 20739, 57145, 1465, 1587, 2146, 2207, 32838, 336 or 52908 substrate, or one which interacts with a non-9805, 2047, 46566, 57749, 577, 20739, 57145, 1465, 1587, 2146, 2207, 32838, 336 or 52908 substrate.

The assays described herein, such as the preceding diagnostic assays or the following assays, can be used to identify a subject having or at risk of developing a pain disorder. A biological sample may be obtained from a subject and tested for the presence or absence of a genetic alteration. For example, such genetic alterations can be detected by ascertaining the existence of at least one of 1) a deletion of one or more nucleotides from an 9805, 2047, 46566, 57749, 577, 20739, 57145, 1465, 1587, 2146, 2207, 32838, 336 or 52908 gene, 2) an addition of one or more nucleotides to an 9805, 2047, 46566, 57749, 577, 20739, 57145, 1465, 1587, 2146, 2207, 32838, 336 or 52908 gene, 3) a substitution of one or more nucleotides of an 9805, 2047, 46566, 57749, 577, 20739, 57145, 1465, 1587, 2146, 2207, 32838, 336 or 52908 gene, 4) a chromosomal rearrangement of an 9805, 2047, 46566, 57749, 577, 20739, 57145, 1465, 1587, 2146, 2207, 32838, 336 or 52908 gene, 5) an alteration in the level of a messenger RNA transcript of an 9805, 2047, 46566, 57749, 577, 20739, 57145, 1465, 1587, 2146, 2207, 32838, 336 or 52908 gene, 6) aberrant modification of an 9805, 2047, 46566, 57749, 577, 20739, 57145, 1465, 1587, 2146, 2207, 32838, 336 or 52908 gene, such as of the methylation pattern of the genomic DNA, 7) the presence of a non-wild type splicing pattern of a messenger RNA transcript of an 9805, 2047, 46566, 57749, 577, 20739, 57145, 1465, 1587, 2146, 2207, 32838, 336 or 52908 gene, 8) a non-wild type level of an 9805, 2047, 46566, 57749, 577, 20739, 57145, 1465, 1587, 2146, 2207, 32838, 336 or 52908-protein, 9) allelic loss of an 9805, 2047, 46566, 57749, 577, 20739, 57145, 1465, 1587, 2146, 2207, 32838, 336 or 52908 gene, and 10) inappropriate post-translational modification of an 9805, 2047, 46566, 57749, 577, 20739, 57145, 1465, 1587, 2146, 2207, 32838, 336 or 52908-protein.

As described herein, there are a large number of assays known in the art which can be used for detecting genetic alterations in an 9805, 2047, 46566, 57749, 577, 20739, 57145, 1465, 1587, 2146, 2207, 32838, 336 or 52908 gene. For example, a genetic alteration in an 9805, 2047, 46566, 57749, 577, 20739, 57145, 1465, 1587, 2146, 2207, 32838, 336 or 52908 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in an 9805, 2047, 46566, 57749, 577, 20739, 57145, 1465, 1587, 2146, 2207, 32838, 336 or 52908 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to an 9805, 2047, 46566, 57749, 577, 20739, 57145, 1465, 1587, 2146, 2207, 32838, 336 or 52908 gene under conditions such that hybridization and amplification of the 9805, 2047, 46566, 57749, 577, 20739, 57145, 1465, 1587, 2146, 2207, 32838, 336 or 52908 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an 9805, 2047, 46566, 57749, 577, 20739, 57145, 1465, 1587, 2146, 2207, 32838, 336 or 52908 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 9805, 2047, 46566, 57749, 577, 20739, 57145, 1465, 1587, 2146, 2207, 32838, 336 or 52908 can be identified by hybridizing biological sample derived and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in 9805, 2047, 46566, 57749, 577, 20739, 57145, 1465, 1587, 2146, 2207, 32838, 336 or 52908 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 9805, 2047, 46566, 57749, 577, 20739, 57145, 1465, 1587, 2146, 2207, 32838, 336 or 52908 gene in a biological sample and detect mutations by comparing the sequence of the 9805, 2047, 46566, 57749, 577, 20739, 57145, 1465, 1587, 2146, 2207, 32838, 336 or 52908 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 9805, 2047, 46566, 57749, 577, 20739, 57145, 1465, 1587, 2146, 2207, 32838, 336 or 52908 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 9805, 2047, 46566, 57749, 577, 20739, 57145, 1465, 1587, 2146, 2207, 32838, 336 or 52908 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 9805, 2047, 46566, 57749, 577, 20739, 57145, 1465, 1587, 2146, 2207, 32838, 336 or 52908 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an 9805, 2047, 46566, 57749, 577, 20739, 57145, 1465, 1587, 2146, 2207, 32838, 336 or 52908 sequence, e.g., a wild-type 9805, 2047, 46566, 57749, 577, 20739, 57145, 1465, 1587, 2146, 2207, 32838, 336 or 52908 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 9805, 2047, 46566, 57749, 577, 20739, 57145, 1465, 1587, 2146, 2207, 32838, 336 or 52908 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 9805, 2047, 46566, 57749, 577, 20739, 57145, 1465, 1587, 2146, 2207, 32838, 336 or 52908 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered an 9805, 2047, 46566, 57749, 577, 20739, 57145, 1465, 1587, 2146, 2207, 32838, 336 or 52908 modulator (e.g., an agonist, antagonist, peptidomimetic, protein, peptide, nucleic acid, or small molecule) to effectively treat a pain disorder.

C. Monitoring of Effects During Clinical Trials

The present invention further provides methods for determining the effectiveness of an 9805, 2047, 46566, 57749, 577, 20739, 57145, 1465, 1587, 2146, 2207, 32838, 336 or 52908 modulator (e.g., an 9805, 2047, 46566, 57749, 577, 20739, 57145, 1465, 1587, 2146, 2207, 32838, 336 or 52908 modulator identified herein) in treating a pain disorder in a subject. For example, the effectiveness of an 9805, 2047, 46566, 57749, 577, 20739, 57145, 1465, 1587, 2146, 2207, 32838, 336 or 52908 modulator in increasing 9805, 2047, 46566, 57749, 577, 20739, 57145, 1465, 1587, 2146, 2207, 32838, 336 or 52908 gene expression, protein levels, or in down regulating 9805, 2047, 46566, 57749, 577, 20739, 57145, 1465, 1587, 2146, 2207, 32838, 336 or 52908 activity, can be monitored in clinical trials of subjects exhibiting decreased 9805, 2047, 46566, 57749, 577, 20739, 57145, 1465, 1587, 2146, 2207, 32838, 336 or 52908 gene expression, protein levels, or upregulated 9805, 2047, 46566, 57749, 577, 20739, 57145, 1465, 1587, 2146, 2207, 32838, 336 or 52908 activity. Alternatively, the effectiveness of an 9805, 2047, 46566, 57749, 577, 20739, 57145, 1465, 1587, 2146, 2207, 32838, 336 or 52908 modulator in decreasing 9805, 2047, 46566, 57749, 577, 20739, 57145, 1465, 1587, 2146, 2207, 32838, 336 or 52908 gene expression, protein levels, or in downregulating 9805, 2047, 46566, 57749, 577, 20739, 57145, 1465, 1587, 2146, 2207, 32838, 336 or 52908 activity, can be monitored in clinical trials of subjects exhibiting increased 9805, 2047, 46566, 57749, 577, 20739, 57145, 1465, 1587, 2146, 2207, 32838, 336 or 52908 gene expression, protein levels, or 9805, 2047, 46566, 57749, 577, 20739, 57145, 1465, 1587, 2146, 2207, 32838, 336 or 52908 activity. In such clinical trials, the expression or activity of an 9805, 2047, 46566, 57749, 577, 20739, 57145, 1465, 1587, 2146, 2207, 32838, 336 or 52908 gene, and preferably, other genes that have been implicated in, for example, a pain disorder can be used as a “read out” or marker of the phenotype of a particular cell.

For example, and not by way of limitation, genes, including 9805, 2047, 46566, 57749, 577, 20739, 57145, 1465, 1587, 2146, 2207, 32838, 336 or 52908, that are modulated in cells by treatment with an agent which modulates 9805, 2047, 46566, 57749, 577, 20739, 57145, 1465, 1587, 2146, 2207, 32838, 336 or 52908 activity (e.g., identified in a screening assay as described herein) can be identified. Thus, to study the effect of agents which modulate 9805, 2047, 46566, 57749, 577, 20739, 57145, 1465, 1587, 2146, 2207, 32838, 336 or 52908 activity on subjects suffering from a pain disorder in, for example, a clinical trial, cells can be isolated and RNA prepared and analyzed for the levels of expression of 9805, 2047, 46566, 57749, 577, 20739, 57145, 1465, 1587, 2146, 2207, 32838, 336 or 52908 and other genes implicated in the pain disorder. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 9805, 2047, 46566, 57749, 577, 20739, 57145, 1465, 1587, 2146, 2207, 32838, 336 or 52908 or other genes. In this way, the gene expression pattern can serve as a marker, indicative of the physiological response of the cells to the agent which modulates 9805, 2047, 46566, 57749, 577, 20739, 57145, 1465, 1587, 2146, 2207, 32838, 336 or 52908 activity. This response state may be determined before, and at various points during treatment of the individual with the agent which modulates 9805, 2047, 46566, 57749, 577, 20739, 57145, 1465, 1587, 2146, 2207, 32838, 336 or 52908 activity.

In a preferred embodiment, the present invention provides a method for monitoring the effectiveness of treatment of a subject with an agent which modulates 9805, 2047, 46566, 57749, 577, 20739, 57145, 1465, 1587, 2146, 2207, 32838, 336 or 52908 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of an 9805, 2047, 46566, 57749, 577, 20739, 57145, 1465, 1587, 2146, 2207, 32838, 336 or 52908 protein, MRNA, or genomic DNA in the pre-administration sample; (iii) obtaining one or more post-administration samples from the subject; (iv) detecting the level of expression or activity of the 9805, 2047, 46566, 57749, 577, 20739, 57145, 1465, 1587, 2146, 2207, 32838, 336 or 52908 protein, mRNA, or genomic DNA in the post-administration samples; (v) comparing the level of expression or activity of the 9805, 2047, 46566, 57749, 577, 20739, 57145, 1465, 1587, 2146, 2207, 32838, 336 or 52908 protein, MRNA, or genomic DNA in the pre-administration sample with the 9805, 2047, 46566, 57749, 577, 20739, 57145, 1465, 1587, 2146, 2207, 32838, 336 or 52908 protein, mRNA, or genomic DNA in the post administration sample or samples; and (vi) altering the administration of the agent to the subject accordingly. For example, increased administration of the agent may be desirable to increase the expression or activity of 9805, 2047, 46566, 57749, 577, 20739, 57145, 1465, 1587, 2146, 2207, 32838, 336 or 52908 to higher levels than detected, i.e., to increase the effectiveness of the agent. Alternatively, decreased administration of the agent may be desirable to decrease expression or activity of 9805, 2047, 46566, 57749, 577, 20739, 57145, 1465, 1587, 2146, 2207, 32838, 336 or 52908 to lower levels than detected, i.e. to decrease the effectiveness of the agent. According to such an embodiment, 9805, 2047, 46566, 57749, 577, 20739, 57145, 1465, 1587, 2146, 2207, 32838, 336 or 52908 expression or activity may be used as an indicator of the effectiveness of an agent, even in the absence of an observable phenotypic response.

IV. Methods of Treatment of Subjects Suffering from Pain Disorders—9805, 2047, 46566, 57749, 577, 20739, 57145, 1465, 1587, 2146, 2207, 32838, 336 or 52908:

The present invention provides for both prophylactic and therapeutic methods of treating a subject, e.g., a human, at risk of (or susceptible to) a pain disorder such as inflammatory pain, chronic pain, neuropathic pain, fibromyalgia, migraine/headache pain, cancer pain, chronic fatigue syndrome, arthritis, complex regional pain syndrome, causalgia, neurodystrophy, or plantar fasciitis. As used herein, “treatment” of a subject includes the application or administration of a therapeutic agent to a subject, or application or administration of a therapeutic agent to a cell or tissue from a subject, who has a disease or disorder, has a symptom of a disease or disorder, or is at risk of (or susceptible to) a disease or disorder, with the purpose to cure, heal, alleviate, relieve, alter, remedy, ameliorate, improve, or affect the disease or disorder, the symptom of the disease or disorder, or the risk of (or susceptibility to) the disease or disorder. As used herein, a “therapeutic agent” includes, but is not limited to, small molecules, peptides, polypeptides, antibodies, ribozymes, and antisense oligonucleotides.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring a subject's prophylactic or therapeutic treatment with either the 9805, 2047, 46566, 57749, 577, 20739, 57145, 1465, 1587, 2146, 2207, 32838, 336 or 52908 molecules of the present invention or 9805, 2047, 46566, 57749, 577, 20739, 57145, 1465, 1587, 2146, 2207, 32838, 336 or 52908 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a pain disorder by administering to the subject an agent which modulates 9805 expression or 9805 activity, e.g., modulation of cAMP production in cells, e.g., neurons. In one aspect, the invention provides a method for preventing in a subject, a pain disorder by administering to the subject an agent which modulates 2047, 46566, 57749, 577, 20739, 57145, 1465, 1587, 2146, 2207, 32838, 336 or 52908 expression or 2047, 46566, 57749, 577, 20739, 57145, 1465, 1587, 2146, 2207, 32838, 336 or 52908 activity, in a cell, e.g., a neuron. Subjects at risk for a pain disorder can be identified by, for example, any or a combination of the diagnostic or prognostic assays described herein. Administration of a prophylactic agent can occur prior to the manifestation of symptoms characteristic of aberrant 9805, 2047, 46566, 57749, 577, 20739, 57145, 1465, 1587, 2146, 2207, 32838, 336 or 52908 expression or activity, such that a pain disorder is prevented or, alternatively, delayed in its progression. Depending on the type of 9805, 2047, 46566, 57749, 577, 20739, 57145, 1465, 1587, 2146, 2207, 32838, 336 or 52908 aberrancy, for example, an 9805, 2047, 46566, 57749, 577, 20739, 57145, 1465, 1587, 2146, 2207, 32838, 336 or 52908 molecule, 9805, 2047, 46566, 57749, 577, 20739, 57145, 1465, 1587, 2146, 2207, 32838, 336 or 52908 agonist or 9805, 2047, 46566, 57749, 577, 20739, 57145, 1465, 1587, 2146, 2207, 32838, 336 or 52908 antagonist agent can be used for treating the subject. The appropriate agent can be determined based on screening assays described herein.

B. Therapeutic Methods

Another aspect of the invention pertains to methods for treating a subject suffering from a pain disorder. These methods involve administering to a subject an agent which modulates 9805, 2047, 46566 or 57749 expression or activity (e.g., an agent identified by a screening assay described herein), or a combination of such agents. In another embodiment, the method involves administering to a subject an 9805, 2047, 46566 or 57749 protein or nucleic acid molecule as therapy to compensate for reduced, aberrant, or unwanted 9805, 2047, 46566 or 57749 expression or activity.

Described herein are methods and compositions whereby pain may be ameliorated. Certain painful disorders are brought about, at least in part, by an excessive level of a gene product, or by the presence of a gene product exhibiting an abnormal or excessive activity. As such, the reduction in the level and/or activity of such gene products would bring about the amelioration of pain. Techniques for the reduction of gene expression levels or the activity of a protein are discussed below.

Alternatively, certain other painful disorders are brought about, at least in part, by the absence or reduction of the level of gene expression, or a reduction in the level of a protein's activity. As such, an increase in the level of gene expression and/or the activity of such proteins would bring about the amelioration of pain.

In some cases, the up-regulation of a gene in a disease state reflects a protective role for that gene product in responding to the disease condition. Enhancement of such a gene's expression, or the activity of the gene product, will reinforce the protective effect it exerts. Some pain states may result from an abnormally low level of activity of such a protective gene. In these cases also, an increase in the level of gene expression and/or the activity of such gene products would bring about the amelioration of pain. Techniques for increasing target gene expression levels or target gene product activity levels are discussed herein.

Accordingly, another aspect of the invention pertains to methods of modulating 577, 20739, 57145, 1465, 1587, 2146, 2207, 32838, 336 or 52908 expression or activity for therapeutic purposes. Accordingly, in an exemplary embodiment, the modulatory method of the invention involves contacting a cell with a 577, 20739, 57145, 1465, 1587, 2146, 2207, 32838, 336 or 52908 or agent that modulates one or more of the activities of 577, 20739, 57145, 1465, 1587, 2146, 2207, 32838, 336 or 52908 protein activity associated with the cell (e.g., a neuron). An agent that modulates 577, 20739, 57145, 1465, 1587, 2146, 2207, 32838, 336 or 52908 protein activity can be an agent as described herein, such as a nucleic acid or a protein, a naturally-occurring target molecule of a 577, 20739, 57145, 1465, 1587, 2146, 2207, 32838, 336 or 52908 protein (e.g., a 577, 20739, 57145, 1465, 1587, 2146, 2207, 32838, 336 or 52908 ligand or substrate), a 577, 20739, 57145, 1465, 1587, 2146, 2207, 32838, 336 or 52908 antibody, a 577, 20739, 57145, 1465, 1587, 2146, 2207, 32838, 336 or 52908 agonist or antagonist, a peptidomimetic of a 577, 20739, 57145, 1465, 1587, 2146, 2207, 32838, 336 or 52908 agonist or antagonist, or other small molecule. In one embodiment, the agent stimulates one or more 577, 20739, 57145, 1465, 1587, 2146, 2207, 32838, 336 or 52908 activities. Examples of such stimulatory agents include active 577, 20739, 57145, 1465, 1587, 2146, 2207, 32838, 336 or 52908 protein and a nucleic acid molecule encoding 577, 20739, 57145, 1465, 1587, 2146, 2207, 32838, 336 or 52908 that has been introduced into the cell. In another embodiment, the agent inhibits one or more 577, 20739, 57145, 1465, 1587, 2146, 2207, 32838, 336 or 52908 activities. Examples of such inhibitory agents include antisense 577, 20739, 57145, 1465, 1587, 2146, 2207, 32838, 336 or 52908 nucleic acid molecules, anti-577, 20739, 57145, 1465, 1587, 2146, 2207, 32838, 336 or 52908 antibodies, and 577, 20739, 57145, 1465, 1587, 2146, 2207, 32838, 336 or 52908 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 577, 20739, 57145, 1465, 1587, 2146, 2207, 32838, 336 or 52908 protein or nucleic acid molecule. In one embodiment, the method involves administering an agent (e.g., an agent identified by a screening assay described herein), or combination of agents that modulates (e.g., upregulates or downregulates) 577, 20739, 57145, 1465, 1587, 2146, 2207, 32838, 336 or 52908 expression or activity. In another embodiment, the method involves administering a 577, 20739, 57145, 1465, 1587, 2146, 2207, 32838, 336 or 52908 protein or nucleic acid molecule as therapy to compensate for reduced, aberrant, or unwanted 577, 20739, 57145, 1465, 1587, 2146, 2207, 32838, 336 or 52908 expression or activity.

Stimulation of 9805, 2047, 46566, 57749, 577, 20739, 57145, 1465, 1587, 2146, 2207, 32838, 336 or 52908 activity is desirable in situations in which 9805, 2047, 46566, 57749, 577, 20739, 57145, 1465, 1587, 2146, 2207, 32838, 336 or 52908 is abnormally downregulated and/or in which increased 9805, 2047, 46566, 57749, 577, 20739, 57145, 1465, 1587, 2146, 2207, 32838, 336 or 52908 activity is likely to have a beneficial effect, i.e., a decrease in thermogenesis, thereby ameliorating a pain disorder such as anorexia or cachexia in a subject. Likewise, inhibition of 9805, 2047, 46566, 57749, 577, 20739, 57145, 1465, 1587, 2146, 2207, 32838, 336 or 52908 activity is desirable in situations in which 9805, 2047, 46566, 57749, 577, 20739, 57145, 1465, 1587, 2146, 2207, 32838, 336 or 52908 is abnormally upregulated and/or in which decreased 9805, 2047, 46566, 57749, 577, 20739, 57145, 1465, 1587, 2146, 2207, 32838, 336 or 52908 activity is likely to have a beneficial effect.

i) Methods for Inhibiting Target Gene Expression, Synthesis, or Activity

As discussed above, genes involved in pain disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate pain. Such molecules may include, but are not limited to, small organic molecules, peptides, antibodies, and the like.

For example, compounds can be administered that compete with endogenous ligand for the 577, 20739, 57145, 1465, 1587, 2146, 2207, 32838, 336 or 52908 protein. The resulting reduction in the amount of ligand-bound 577, 20739, 57145, 1465, 1587, 2146, 2207, 32838, 336 or 52908 protein will modulate endothelial cell physiology. Compounds that can be particularly useful for this purpose include, for example, soluble proteins or peptides, such as peptides comprising one or more of the extracellular domains, or portions and/or analogs thereof, of the 577, 20739, 57145, 1465, 1587, 2146, 2207, 32838, 336 or 52908 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 577, 20739, 57145, 1465, 1587, 2146, 2207, 32838, 336 or 52908 receptor site, but do not activate the protein, (e.g., receptor-ligand antagonists) can be effective in inhibiting 577, 20739, 57145, 1465, 1587, 2146, 2207, 32838, 336 or 52908 protein activity.

Further, antisense and ribozyme molecules which inhibit expression of the 577, 20739, 57145, 1465, 1587, 2146, 2207, 32838, 336 or 52908 gene may also be used in accordance with the invention to inhibit aberrant 577, 20739, 57145, 1465, 1587, 2146, 2207, 32838, 336 or 52908 gene activity. Still further, triple helix molecules may be utilized in inhibiting aberrant 577, 20739, 57145, 1465, 1587, 2146, 2207, 32838, 336 or 52908 gene activity.

The antisense nucleic acid molecules used in the methods of the invention are typically administered to a subject or generated in situ such that they hybridize with or bind to cellular mRNA and/or genomic DNA encoding a 577, 20739, 57145, 1465, 1587, 2146, 2207, 32838, 336 or 52908 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, an antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 577, 20739, 57145, 1465, 1587, 2146, 2207, 32838, 336 or 52908 mRNA transcripts to thereby inhibit translation of 577, 20739, 57145, 1465, 1587, 2146, 2207, 32838, 336 or 52908 mRNA. A ribozyme having specificity for a 577, 20739, 57145, 1465, 1587, 2146, 2207, 32838, 336 or 52908-encoding nucleic acid can be designed based upon the nucleotide sequence of a 577, 20739, 57145, 1465, 1587, 2146, 2207, 32838, 336 or 52908 cDNA disclosed herein (i.e., SEQ ID NO:12, 13, 15, 16, 18, 19, 21, 22, 24, 25, 27, 28, 30, 31, 33, 34, 36, 37, 39 or 40). For example, a derivative of a Tetrahymena L-19 IVS RNA can be constructed in which the nucleotide sequence of the active site is complementary to the nucleotide sequence to be cleaved in a 577, 20739, 57145, 1465, 1587, 2146, 2207, 32838, 336 or 52908-encoding mRNA (see, for example, Cech et al. U.S. Pat. No. 4,987,071; and Cech et al. U.S. Pat. No. 5,116,742). Alternatively, 577, 20739, 57145, 1465, 1587, 2146, 2207, 32838, 336 or 52908 MRNA can be used to select a catalytic RNA having a specific ribonuclease activity from a pool of RNA molecules (see, for example, Bartel, D. and Szostak, J. W. (1993) Science 261:1411-1418).

577, 20739, 57145, 1465, 1587, 2146, 2207, 32838, 336 or 52908 gene expression can also be inhibited by targeting nucleotide sequences complementary to the regulatory region of the 577, 20739, 57145, 1465, 1587, 2146, 2207, 32838, 336 or 52908 (e.g., the 577, 20739, 57145, 1465, 1587, 2146, 2207, 32838, 336 or 52908 promoter and/or enhancers) to form triple helical structures that prevent transcription of the 577, 20739, 57145, 1465, 1587, 2146, 2207, 32838, 336 or 52908 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the 577, 20739, 57145, 1465, 1587, 2146, 2207, 32838, 336 or 52908 protein and interfere with its activity may also be used to modulate or inhibit 577, 20739, 57145, 1465, 1587, 2146, 2207, 32838, 336 or 52908 protein function. Such antibodies may be generated using standard techniques described herein, against the 577, 20739, 57145, 1465, 1587, 2146, 2207, 32838, 336 or 52908 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 577, 20739, 57145, 1465, 1587, 2146, 2207, 32838, 336 or 52908 protein. Antibodies that are specific for one or more extracellular domains of the 577, 20739, 57145, 1465, 1587, 2146, 2207, 32838, 336 or 52908 protein, for example, and that interfere with its activity, are particularly useful in treating pain or a painful disorder. Such antibodies are especially efficient because they can access the target domains directly from the bloodstream. Any of the administration techniques described below which are appropriate for peptide administration may be utilized to effectively administer inhibitory target gene antibodies to their site of action.

ii) Methods for Restoring or Enhancing Target Gene Activity

Genes that cause pain may be underexpressed within pain disease situations. Alternatively, the activity of the protein products of such genes may be decreased, leading to the development of pain. Such down-regulation of gene expression or decrease of protein activity might have a causative or exacerbating effect on the disease state.

In some cases, genes that are up-regulated in the disease state might be exerting a protective effect. A variety of techniques may be used to increase the expression, synthesis, or activity of genes and/or proteins that exert a protective effect in response to pain conditions.

Described in this section are methods whereby the level 577, 20739, 57145, 1465, 1587, 2146, 2207, 32838, 336 or 52908 activity may be increased to levels wherein pain are ameliorated. The level of 577, 20739, 57145, 1465, 1587, 2146, 2207, 32838, 336 or 52908 activity may be increased, for example, by either increasing the level of 577, 20739, 57145, 1465, 1587, 2146, 2207, 32838, 336 or 52908 gene expression or by increasing the level of active 577, 20739, 57145, 1465, 1587, 2146, 2207, 32838, 336 or 52908 protein which is present.

For example, a 577, 20739, 57145, 1465, 1587, 2146, 2207, 32838, 336 or 52908 protein, at a level sufficient to ameliorate pain may be administered to a patient exhibiting such symptoms. Any of the techniques discussed below may be used for such administration. One of skill in the art will readily know how to determine the concentration of effective, non-toxic doses of the 577, 20739, 57145, 1465, 1587, 2146, 2207, 32838, 336 or 52908 protein, utilizing techniques such as those described below.

Additionally, RNA sequences encoding a 577, 20739, 57145, 1465, 1587, 2146, 2207, 32838, 336 or 52908 protein may be directly administered to a patient exhibiting pain, at a concentration sufficient to produce a level of 577, 20739, 57145, 1465, 1587, 2146, 2207, 32838, 336 or 52908 protein such that pain are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a 577, 20739, 57145, 1465, 1587, 2146, 2207, 32838, 336 or 52908 gene, or a portion thereof, that directs the production of a normal 577, 20739, 57145, 1465, 1587, 2146, 2207, 32838, 336 or 52908 protein with 577, 20739, 57145, 1465, 1587, 2146, 2207, 32838, 336 or 52908 function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 577, 20739, 57145, 1465, 1587, 2146, 2207, 32838, 336 or 52908 gene sequences into human cells.

Cells, preferably, autologous cells, containing 577, 20739, 57145, 1465, 1587, 2146, 2207, 32838, 336 or 52908 expressing gene sequences may then be introduced or reintroduced into the subject at positions which allow for the amelioration of pain. Such cell replacement techniques may be preferred, for example, when the gene product is a secreted, extracellular gene product.

C. Pharmaceutical Compositions

Another aspect of the invention pertains to methods for treating a subject suffering from a disease. These methods involve administering to a subject an agent which modulates 9805, 2047, 46566, 57749, 577, 20739, 57145, 1465, 1587, 2146, 2207, 32838, 336 or 52908 expression or activity (e.g., an agent identified by a screening assay described herein), or a combination of such agents. In another embodiment, the method involves administering to a subject a 9805, 2047, 46566, 57749, 577, 20739, 57145, 1465, 1587, 2146, 2207, 32838, 336 or 52908 protein or nucleic acid molecule as therapy to compensate for reduced, aberrant, or unwanted 9805, 2047, 46566, 57749, 577, 20739, 57145, 1465, 1587, 2146, 2207, 32838, 336 or 52908 expression or activity.

Stimulation of 9805, 2047, 46566, 57749, 577, 20739, 57145, 1465, 1587, 2146, 2207, 32838, 336 or 52908 activity is desirable in situations in which 9805, 2047, 46566, 57749, 577, 20739, 57145, 1465, 1587, 2146, 2207, 32838, 336 or 52908 is abnormally downregulated and/or in which increased 9805, 2047, 46566, 57749, 577, 20739, 57145, 1465, 1587, 2146, 2207, 32838, 336 or 52908 activity is likely to have a beneficial effect. Likewise, inhibition of 9805, 2047, 46566, 57749, 577, 20739, 57145, 1465, 1587, 2146, 2207, 32838, 336 or 52908 activity is desirable in situations in which 9805, 2047, 46566, 57749, 577, 20739, 57145, 1465, 1587, 2146, 2207, 32838, 336 or 52908 is abnormally upregulated and/or in which decreased 9805, 2047, 46566, 57749, 577, 20739, 57145, 1465, 1587, 2146, 2207, 32838, 336 or 52908 activity is likely to have a beneficial effect.

The agents which modulate 9805, 2047, 46566, 57749, 577, 20739, 57145, 1465, 1587, 2146, 2207, 32838, 336 or 52908 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 9805, 2047, 46566, 57749, 577, 20739, 57145, 1465, 1587, 2146, 2207, 32838, 336 or 52908 activity (e.g., a fragment of an 9805, 2047, 46566, 57749, 577, 20739, 57145, 1465, 1587, 2146, 2207, 32838, 336 or 52908 protein or an anti-9805, 2047, 46566, 57749, 577, 20739, 57145, 1465, 1587, 2146, 2207, 32838, 336 or 52908 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 9805, 2047, 46566, 57749, 577, 20739, 57145, 1465, 1587, 2146, 2207, 32838, 336 or 52908 activity can also be prepared in the form of suppositories (e.g., with conventional suppository bases such as cocoa butter and other glycerides) or retention enemas for rectal delivery.

In one embodiment, the agents that modulate 9805, 2047, 46566, 57749, 577, 20739, 57145, 1465, 1587, 2146, 2207, 32838, 336 or 52908 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 9805, 2047, 46566, 57749, 577, 20739, 57145, 1465, 1587, 2146, 2207, 32838, 336 or 52908 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 9805, 2047, 46566, 57749, 577, 20739, 57145, 1465, 1587, 2146, 2207, 32838, 336 or 52908 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

V. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 9805, 2047, 46566, 57749, 577, 20739, 57145, 1465, 1587, 2146, 2207, 32838, 336 or 52908 activity, as well as tailoring the dosage and/or therapeutic regimen of treatment with an agent which modulates 9805, 2047, 46566, 57749, 577, 20739, 57145, 1465, 1587, 2146, 2207, 32838, 336 or 52908 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In geneneral, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., an 9805, 2047, 46566, 57749, 577, 20739, 57145, 1465, 1587, 2146, 2207, 32838, 336 or 52908 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., an 9805, 2047, 46566, 57749, 577, 20739, 57145, 1465, 1587, 2146, 2207, 32838, 336 or 52908 molecule or 9805, 2047, 46566, 57749, 577, 20739, 57145, 1465, 1587, 2146, 2207, 32838, 336 or 52908 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a pain disorder with an agent which modulates 9805, 2047, 46566, 57749, 577, 20739, 57145, 1465, 1587, 2146, 2207, 32838, 336 or 52908 activity.

VI. Recombinant Expression Vectors and Host Cells Used in the Methods of the Invention—9805, 2047, 46566, 57749, 577, 20739, 57145, 1465, 1587, 2146, 2207, 32838, 336 or 52908

The methods of the invention (e.g., the screening assays described herein) include the use of vectors, preferably expression vectors, containing a nucleic acid encoding an 9805, 2047, 46566, 57749, 577, 20739, 57145, 1465, 1587, 2146, 2207, 32838, 336 or 52908 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 9805, 2047, 46566, 57749, 577, 20739, 57145, 1465, 1587, 2146, 2207, 32838, 336 or 52908 proteins, mutant forms of 9805, 2047, 46566, 57749, 577, 20739, 57145, 1465, 1587, 2146, 2207, 32838, 336 or 52908 proteins, fusion proteins, and the like).

The recombinant expression vectors to be used in the methods of the invention can be designed for expression of 9805, 2047, 46566, 57749, 577, 20739, 57145, 1465, 1587, 2146, 2207, 32838, 336 or 52908 proteins in prokaryotic or eukaryotic cells. For example, 9805, 2047, 46566, 57749, 577, 20739, 57145, 1465, 1587, 2146, 2207, 32838, 336 or 52908 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 9805, 2047, 46566, 57749, 577, 20739, 57145, 1465, 1587, 2146, 2207, 32838, 336 or 52908 activity assays, (e.g., direct assays or competitive assays described in detail below), or to generate antibodies specific for 9805, 2047, 46566, 57749, 577, 20739, 57145, 1465, 1587, 2146, 2207, 32838, 336 or 52908 proteins. In a preferred embodiment, an 9805, 2047, 46566, 57749, 577, 20739, 57145, 1465, 1587, 2146, 2207, 32838, 336 or 52908 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 9805, 2047, 46566, 57749, 577, 20739, 57145, 1465, 1587, 2146, 2207, 32838, 336 or 52908 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which an 9805, 2047, 46566, 57749, 577, 20739, 57145, 1465, 1587, 2146, 2207, 32838, 336 or 52908 nucleic acid molecule of the invention is introduced, e.g., an 9805, 2047, 46566, 57749, 577, 20739, 57145, 1465, 1587, 2146, 2207, 32838, 336 or 52908 nucleic acid molecule within a recombinant expression vector or an 9805, 2047, 46566, 57749, 577, 20739, 57145, 1465, 1587, 2146, 2207, 32838, 336 or 52908 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an 9805, 2047, 46566, 57749, 577, 20739, 57145, 1465, 1587, 2146, 2207, 32838, 336 or 52908 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) an 9805, 2047, 46566, 57749, 577, 20739, 57145, 1465, 1587, 2146, 2207, 32838, 336 or 52908 protein. Accordingly, the invention further provides methods for producing an 9805, 2047, 46566, 57749, 577, 20739, 57145, 1465, 1587, 2146, 2207, 32838, 336 or 52908 protein using the host cells of the invention. In one embodiment, the method comprises culturing the host cell of the invention (into which a recombinant expression vector encoding an 9805, 2047, 46566, 57749, 577, 20739, 57145, 1465, 1587, 2146, 2207, 32838, 336 or 52908 protein has been introduced) in a suitable medium such that an 9805, 2047, 46566, 57749, 577, 20739, 57145, 1465, 1587, 2146, 2207, 32838, 336 or 52908 protein is produced. In another embodiment, the method further comprises isolating an 9805, 2047, 46566, 57749, 577, 20739, 57145, 1465, 1587, 2146, 2207, 32838, 336 or 52908 protein from the medium or the host cell.

VII. Isolated Nucleic Acid Molecules Used in the Methods of the Invention—9805, 2047, 46566, 57749, 577, 20739, 57145, 1465, 1587, 2146, 2207, 32838, 336 or 52908

The DNA sequence of the isolated human 9805 gene and the predicted amino acid sequence of the human 9805 polypeptide (GenBank Accession No. P51828) are shown in SEQ ID NOs:1 and 2, respectively.

The cDNA sequence of the isolated human 2047 gene and the predicted amino acid sequence of the human 2047 polypeptide are shown in SEQ ID NOs:3 and 4, respectively. The coding region without the 5′ or 3′ untranslated regions of the human 2047 gene is shown in SEQ ID NO:5.

The cDNA sequence of the isolated human 46566 gene and the predicted amino acid sequence of the human 46566 polypeptide are shown in SEQ ID NOs:6 and 7, respectively. The coding region without the 5′ or 3′ untranslated regions of the human 2047 gene is shown in SEQ ID NO:8.

The DNA sequence of the isolated human 57749 gene and the predicted amino acid sequence of the human 57749 polypeptide (GenBank Accession No: AF097366) are shown in SEQ ID NOs:9 and 10, respectively.

The methods of the invention include the use of isolated nucleic acid molecules that encode 9805, 2047, 46566, 57749, 577, 20739, 57145, 1465, 1587, 2146, 2207, 32838, 336 or 52908 proteins or biologically active portions thereof, as well as nucleic acid fragments sufficient for use as hybridization probes to identify 9805, 2047, 46566, 57749, 577, 20739, 57145, 1465, 1587, 2146, 2207, 32838, 336 or 52908-encoding nucleic acid molecules (e.g., 9805, 2047, 46566, 57749, 577, 20739, 57145, 1465, 1587, 2146, 2207, 32838, 336 or 52908 mRNA) and fragments for use as PCR primers for the amplification or mutation of 9805, 2047, 46566, 57749, 577, 20739, 57145, 1465, 1587, 2146, 2207, 32838, 336 or 52908 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, or a portion thereof, can be isolated using standard molecular biology techniques and the sequence information provided herein. Using all or portion of the nucleic acid sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40 as a hybridization probe, 9805, 2047, 46566, 57749, 577, 20739, 57145, 1465, 1587, 2146, 2207, 32838, 336 or 52908 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40 can be isolated by the polymerase chain reaction (PCR) using synthetic oligonucleotide primers designed based upon the sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 9805, 2047, 46566, 57749, 577, 20739, 57145, 1465, 1587, 2146, 2207, 32838, 336 or 52908 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, a complement of the nucleotide sequence shown in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, oraportion of any of these nucleotide sequences. A nucleic acid molecule which is complementary to the nucleotide sequence shown in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, is one which is sufficiently complementary to the nucleotide sequence shown in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40 such that it can hybridize to the nucleotide sequence shown in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99.1% 99.2%, 99.3%, 99.4%, 99.5%, 99.6%, 99.7%, 99.8%, 99.9% or more identical to the entire length of the nucleotide sequence shown in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, or a portion of any of this nucleotide sequence.

Moreover, the nucleic acid molecules used in the methods of the invention can comprise only a portion of the nucleic acid sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, for example, a fragment which can be used as a probe or primer or a fragment encoding a portion of an 9805, 2047, 46566, 57749, 577, 20739, 57145, 1465, 1587, 2146, 2207, 32838, 336 or 52908 protein, e.g., a biologically active portion of an 9805, 2047, 46566, 57749, 577, 20739, 57145, 1465, 1587, 2146, 2207, 32838, 336 or 52908 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, 75, 100, 200 or more consecutive nucleotides of a sense sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40 or an anti-sense sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, or of a naturally occurring allelic variant or mutant of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40. In one embodiment, a nucleic acid molecule used in the methods of the present invention comprises a nucleotide sequence which is greater than 50, 50-100, 100-200, 200-300, 300-400, 400-500, 500-600, 600-700, 700-800, 800-900, 900-1000, 1000-1500, 1500-2000, 2000-3000 or more nucleotides in length and hybridizes under stringent hybridization conditions to a nucleic acid molecule of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× or 6× sodium chloride/sodium citrate (SSC), at about 65-70° C. (or hybridization in 4×SSC plus 50% formamide at about 42-50° C.) followed by one or more washes in 1×SSC, at about 65-70° C. A further preferred, non-limiting example of stringent hybridization conditions includes hybridization at 6×SSC at 45° C., followed by one or more washes in 0.2×SSC, 0.1% SDS at 65° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4× or 6×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an 9805, 2047, 46566, 57749, 577, 20739, 57145, 1465, 1587, 2146, 2207, 32838, 336 or 52908 protein, such as by measuring a level of an 9805, 2047, 46566, 57749, 577, 20739, 57145, 1465, 1587, 2146, 2207, 32838, 336 or 52908-encoding nucleic acid in a sample of cells from a subject e.g., detecting 9805, 2047, 46566, 57749, 577, 20739, 57145, 1465, 1587, 2146, 2207, 32838, 336 or 52908 mRNA levels or determining whether a genomic 9805, 2047, 46566, 57749, 577, 20739, 57145, 1465, 1587, 2146, 2207, 32838, 336 or 52908 gene has been mutated or deleted.

The methods of the invention further encompass the use of nucleic acid molecules that differ from the nucleotide sequence shown in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40 due to degeneracy of the genetic code and thus encode the same 9805, 2047, 46566, 57749, 577, 20739, 57145, 1465, 1587, 2146, 2207, 32838, 336 or 52908 proteins as those encoded by the nucleotide sequence shown in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40. In another embodiment, an isolated nucleic acid molecule included in the methods of the invention has a nucleotide sequence encoding a protein having an amino acid sequence shown in SEQ ID NO:2, 4, 7, 10, 14, 17, 20, 23, 26, 29, 32, 35, 38 or 41.

The methods of the invention further include the use of allelic variants of human 9805, 2047, 46566, 57749, 577, 20739, 57145, 1465, 1587, 2146, 2207, 32838, 336 or 52908, e.g., functional and non-functional allelic variants. Functional allelic variants are naturally occurring amino acid sequence variants of the human 9805, 2047, 46566, 57749, 577, 20739, 57145, 1465, 1587, 2146, 2207, 32838, 336 or 52908 protein that maintain an 9805, 2047, 46566, 57749, 577, 20739, 57145, 1465, 1587, 2146, 2207, 32838, 336 or 52908 activity. Functional allelic variants will typically contain only conservative substitution of one or more amino acids of SEQ ID NO:2, 4, 7, 10, 14, 17, 20, 23, 26, 29, 32, 35, 38 or 41, or substitution, deletion or insertion of non-critical residues in non-critical regions of the protein.

Non-functional allelic variants are naturally occurring amino acid sequence variants of the human 9805, 2047, 46566, 57749, 577, 20739, 57145, 1465, 1587, 2146, 2207, 32838, 336 or 52908 protein that do not have an 9805, 2047, 46566, 57749, 577, 20739, 57145, 1465, 1587, 2146, 2207, 32838, 336 or 52908 activity. Non-functional allelic variants will typically contain a non-conservative substitution, deletion, or insertion or premature truncation of the amino acid sequence of SEQ ID NO:2, 4, 7, 10, 14, 17, 20, 23, 26, 29, 32, 35, 38 or 41, or a substitution, insertion or deletion in critical residues or critical regions of the protein.

The methods of the present invention may further use non-human orthologues of the human 9805, 2047, 46566, 57749, 577, 20739, 57145, 1465, 1587, 2146, 2207, 32838, 336 or 52908 protein. Orthologues of the human 9805, 2047, 46566, 57749, 577, 20739, 57145, 1465, 1587, 2146, 2207, 32838, 336 or 52908 protein are proteins that are isolated from non-human organisms and possess the same 9805, 2047, 46566, 57749, 577, 20739, 57145, 1465, 1587, 2146, 2207, 32838, 336 or 52908 activity.

The methods of the present invention further include the use of nucleic acid molecules comprising the nucleotide sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 9805, 2047, 46566, 57749, 577, 20739, 57145, 1465, 1587, 2146, 2207, 32838, 336 or 52908 (e.g., the sequence of SEQ ID NO:2, 4, 7, 10, 14, 17, 20, 23, 26, 29, 32, 35, 38 or 41) without altering the biological activity, whereas an “essential” amino acid residue is required for biological activity. For example, amino acid residues that are conserved among the 9805, 2047, 46566, 57749, 577, 20739, 57145, 1465, 1587, 2146, 2207, 32838, 336 or 52908 proteins of the present invention and other members of the short-chain dehydrogenase family are not likely to be amenable to alteration.

Mutations can be introduced into SEQ ID NO:1, 3, 5, 6, 8, 9, 11, 12, 13, 15, 16, 18, 19, 21, 22, 24, 25, 27, 28, 30, 31, 33, 34, 36, 37, 39 or 40 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an 9805, 2047, 46566, 57749, 577, 20739, 57145, 1465, 1587, 2146, 2207, 32838, 336 or 52908 protein is preferably replaced with another amino acid residue from the same side chain family. Alternatively, in another embodiment, mutations can be introduced randomly along all or part of an 9805, 2047, 46566, 57749, 577, 20739, 57145, 1465, 1587, 2146, 2207, 32838, 336 or 52908 coding sequence, such as by saturation mutagenesis, and the resultant mutants can be screened for 9805, 2047, 46566, 57749, 577, 20739, 57145, 1465, 1587, 2146, 2207, 32838, 336 or 52908 biological activity to identify mutants that retain activity. Following mutagenesis of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40, the encoded protein can be expressed recombinantly and the activity of the protein can be determined using an assay described herein.

Another aspect of the invention pertains to the use of isolated nucleic acid molecules which are antisense to the nucleotide sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40. An “antisense” nucleic acid comprises a nucleotide sequence which is complementary to a “sense” nucleic acid encoding a protein, e.g., complementary to the coding strand of a double-stranded cDNA molecule or complementary to an MRNA sequence. Accordingly, an antisense nucleic acid can hydrogen bond to a sense nucleic acid. The antisense nucleic acid can be complementary to an entire 9805, 2047, 46566, 57749, 577, 20739, 57145, 1465, 1587, 2146, 2207, 32838, 336 or 52908 coding strand, or to only a portion thereof. In one embodiment, an antisense nucleic acid molecule is antisense to a “coding region” of the coding strand of a nucleotide sequence encoding an 9805, 2047, 46566, 57749, 577, 20739, 57145, 1465, 1587, 2146, 2207, 32838, 336 or 52908. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 9805, 2047, 46566, 57749, 577, 20739, 57145, 1465, 1587, 2146, 2207, 32838, 336 or 52908. The term “noncoding region” refers to 5′ and 3′ sequences which flank the coding region that are not translated into amino acids (also referred to as 5′ and 3′ untranslated regions).

Given the coding strand sequences encoding 9805, 2047, 46566, 57749, 577, 20739, 57145, 1465, 1587, 2146, 2207, 32838, 336 or 52908 disclosed herein, antisense nucleic acids of the invention can be designed according to the rules of Watson and Crick base pairing. The antisense nucleic acid molecule can be complementary to the entire coding region of 9805, 2047, 46566, 57749, 577, 20739, 57145, 1465, 1587, 2146, 2207, 32838, 336 or 52908 mRNA, but more preferably is an oligonucleotide which is antisense to only a portion of the coding or noncoding region of 9805, 2047, 46566, 57749, 577, 20739, 57145, 1465, 1587, 2146, 2207, 32838, 336 or 52908 mRNA. For example, the antisense oligonucleotide can be complementary to the region surrounding the translation start site of 9805, 2047, 46566, 57749, 577, 20739, 57145, 1465, 1587, 2146, 2207, 32838, 336 or 52908 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding an 9805, 2047, 46566, 57749, 577, 20739, 57145, 1465, 1587, 2146, 2207, 32838, 336 or 52908 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave 9805, 2047, 46566, 57749, 577, 20739, 57145, 1465, 1587, 2146, 2207, 32838, 336 or 52908 mRNA transcripts to thereby inhibit translation of 9805, 2047, 46566, 57749, 577, 20739, 57145, 1465, 1587, 2146, 2207, 32838, 336 or 52908 mRNA. A ribozyme having specificity for an 9805, 2047, 46566, 57749, 577, 20739, 57145, 1465, 1587, 2146, 2207, 32838, 336 or 52908-encoding nucleic acid can be designed based upon the nucleotide sequence of an 9805, 2047, 46566, 57749, 577, 20739, 57145, 1465, 1587, 2146, 2207, 32838, 336 or 52908 cDNA disclosed herein (i.e., SEQ ID NO:1, 3, 5, 6, 8, 9, 11, 12, 13, 15, 16, 18, 19, 21, 22, 24, 25, 27, 28, 30, 31, 33, 34, 36, 37, 39 or 40). For example, a derivative of a Tetrahymena L-19 IVS RNA can be constructed in which the nucleotide sequence of the active site is complementary to the nucleotide sequence to be cleaved in an 9805, 2047, 46566, 57749, 577, 20739, 57145, 1465, 1587, 2146, 2207, 32838, 336 or 52908-encoding mRNA. See, e.g., Cech et al. U.S. Pat. No. 4,987,071; and Cech et al. U.S. Pat. No. 5,116,742. Alternatively, 9805, 2047, 46566, 57749, 577, 20739, 57145, 1465, 1587, 2146, 2207, 32838, 336 or 52908 mRNA can be used to select a catalytic RNA having a specific ribonuclease activity from a pool of RNA molecules. See, e.g., Bartel, D. and Szostak, J. W. (1993) Science 261:1411-1418.

Alternatively, 9805, 2047, 46566, 57749, 577, 20739, 57145, 1465, 1587, 2146, 2207, 32838, 336 or 52908 gene expression can be inhibited by targeting nucleotide sequences complementary to the regulatory region of the 9805, 2047, 46566, 57749, 577, 20739, 57145, 1465, 1587, 2146, 2207, 32838, 336 or 52908 (e.g., the 9805, 2047, 46566, 57749, 577, 20739, 57145, 1465, 1587, 2146, 2207, 32838, 336 or 52908 promoter and/or enhancers) to form triple helical structures that prevent transcription of the 9805, 2047, 46566, 57749, 577, 20739, 57145, 1465, 1587, 2146, 2207, 32838, 336 or 52908 gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioessays 14(12):807-15.

In yet another embodiment, the 9805, 2047, 46566, 57749, 577, 20739, 57145, 1465, 1587, 2146, 2207, 32838, 336 or 52908 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. and Nielsen (1996) supra and Perry-O'Keefe et al. (1996) Proc. Natl. Acad. Sci. USA 93:14670-675.

PNAs of 9805, 2047, 46566, 57749, 577, 20739, 57145, 1465, 1587, 2146, 2207, 32838, 336 or 52908 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 9805, 2047, 46566, 57749, 577, 20739, 57145, 1465, 1587, 2146, 2207, 32838, 336 or 52908 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup and Nielsen (1996) supra)); or as probes or primers for DNA sequencing or hybridization (Hyrup and Nielsen (1996) supra; Perry-O'Keefe et al. (1996) supra).

In another embodiment, PNAs of 9805, 2047, 46566, 57749, 577, 20739, 57145, 1465, 1587, 2146, 2207, 32838, 336 or 52908 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 9805, 2047, 46566, 57749, 577, 20739, 57145, 1465, 1587, 2146, 2207, 32838, 336 or 52908 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup and Nielsen (1996) supra). The synthesis of PNA-DNA chimeras can be performed as described in Hyrup and Nielsen (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acids Res. 17: 5973-88). PNA monomers are then coupled in a stepwise manner to produce a chimeric molecule with a 5′ PNA segment and a 3′ DNA segment (Finn et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Biotechniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

VIII. Isolated 9805, 2047, 46566, 57749, 577, 20739, 57145, 1465, 1587, 2146, 2207, 32838, 336 or 52908 Proteins and Anti-9805, 2047, 46566, 57749, 577, 20739, 57145, 1465, 1587, 2146, 2207, 32838, 336 or 52908 Antibodies Used in the Methods of the Invention

The methods of the invention include the use of isolated 9805, 2047, 46566, 57749, 577, 20739, 57145, 1465, 1587, 2146, 2207, 32838, 336 or 52908 proteins, and biologically active portions thereof, as well as polypeptide fragments suitable for use as immunogens to raise anti-9805, 2047, 46566, 57749, 577, 20739, 57145, 1465, 1587, 2146, 2207, 32838, 336 or 52908 antibodies. In one embodiment, native 9805, 2047, 46566, 57749, 577, 20739, 57145, 1465, 1587, 2146, 2207, 32838, 336 or 52908 proteins can be isolated from cells or tissue sources by an appropriate purification scheme using standard protein purification techniques. In another embodiment, 9805, 2047, 46566, 57749, 577, 20739, 57145, 1465, 1587, 2146, 2207, 32838, 336 or 52908 proteins are produced by recombinant DNA techniques. Alternative to recombinant expression, an 9805, 2047, 46566, 57749, 577, 20739, 57145, 1465, 1587, 2146, 2207, 32838, 336 or 52908 protein or polypeptide can be synthesized chemically using standard peptide synthesis techniques.

As used herein, a “biologically active portion” of an 9805, 2047, 46566, 57749, 577, 20739, 57145, 1465, 1587, 2146, 2207, 32838, 336 or 52908 protein includes a fragment of an 9805, 2047, 46566, 57749, 577, 20739, 57145, 1465, 1587, 2146, 2207, 32838, 336 or 52908 protein having an 9805, 2047, 46566, 57749, 577, 20739, 57145, 1465, 1587, 2146, 2207, 32838, 336 or 52908 activity. Biologically active portions of an 9805, 2047, 46566, 57749, 577, 20739, 57145, 1465, 1587, 2146, 2207, 32838, 336 or 52908 protein include peptides comprising amino acid sequences sufficiently identical to or derived from the amino acid sequence of the 9805, 2047, 46566, 57749, 577, 20739, 57145, 1465, 1587, 2146, 2207, 32838, 336 or 52908 protein, e.g., the amino acid sequence shown in SEQ ID NO:2, 4, 7, 10, 14, 17, 20, 23, 26, 29, 32, 35, 38 or 41, which include fewer amino acids than the full length 9805, 2047, 46566, 57749, 577, 20739, 57145, 1465, 1587, 2146, 2207, 32838, 336 or 52908 proteins, and exhibit at least one activity of an 9805, 2047, 46566, 57749, 577, 20739, 57145, 1465, 1587, 2146, 2207, 32838, 336 or 52908 protein. Typically, biologically active portions comprise a domain or motif with at least one activity of the 9805, 2047, 46566, 57749, 577, 20739, 57145, 1465, 1587, 2146, 2207, 32838, 336 or 52908 protein. A biologically active portion of an 9805, 2047, 46566, 57749, 577, 20739, 57145, 1465, 1587, 2146, 2207, 32838, 336 or 52908 protein can be a polypeptide which is, for example, 25, 50, 75, 100, 125, 150, 175, 200, 250, 300 or more amino acids in length. Biologically active portions of an 9805, 2047, 46566, 57749, 577, 20739, 57145, 1465, 1587, 2146, 2207, 32838, 336 or 52908 protein can be used as targets for developing agents which modulate an 9805, 2047, 46566, 57749, 577, 20739, 57145, 1465, 1587, 2146, 2207, 32838, 336 or 52908 activity.

In a preferred embodiment, the 9805, 2047, 46566, 57749, 577, 20739, 57145, 1465, 1587, 2146, 2207, 32838, 336 or 52908 protein used in the methods of the invention has an amino acid sequence shown in SEQ ID NO:2, 4, 7, 10, 14, 17, 20, 23, 26, 29, 32, 35, 38 or 41. In other embodiments, the 9805, 2047, 46566, 57749, 577, 20739, 57145, 1465, 1587, 2146, 2207, 32838, 336 or 52908 protein is substantially identical to SEQ ID NO:2, 4, 7, 10, 14, 17, 20, 23, 26, 29, 32, 35, 38 or 41, and retains the functional activity of the protein of SEQ ID NO:2, 4, 7, 10, 14, 17, 20, 23, 26, 29, 32, 35, 38 or 41, yet differs in amino acid sequence due to natural allelic variation or mutagenesis, as described in detail in subsection V above. Accordingly, in another embodiment, the 9805, 2047, 46566, 57749, 577, 20739, 57145, 1465, 1587, 2146, 2207, 32838, 336 or 52908 protein used in the methods of the invention is a protein which comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to SEQ ID NO:2, 4, 7, 10, 14, 17, 20, 23, 26, 29, 32, 35, 38or 41.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 9805, 2047, 46566, 57749, 577, 20739, 57145, 1465, 1587, 2146, 2207, 32838, 336 or 52908 amino acid sequence of SEQ ID NO:2, 4, 7, 10, 14, 17, 20, 23, 26, 29, 32, 35, 38 or 41 having 311 amino acid residues, at least 93, preferably at least 124, more preferably at least 156, even more preferably at least 187, and even more preferably at least 218, 249, 280 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available at www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use 9805, 2047, 46566, 57749, 577, 20739, 57145, 1465, 1587, 2146, 2207, 32838, 336 or 52908 chimeric or fusion proteins. As used herein, an 9805, 2047, 46566, 57749, 577, 20739, 57145, 1465, 1587, 2146, 2207, 32838, 336 or 52908 “chimeric protein” or “fusion protein” comprises an 9805, 2047, 46566, 57749, 577, 20739, 57145, 1465, 1587, 2146, 2207, 32838, 336 or 52908 polypeptide operatively linked to a non-9805, 2047, 46566, 57749, 577, 20739, 57145, 1465, 1587, 2146, 2207, 32838, 336 or 52908 polypeptide. A “9805, 2047, 46566, 57749, 577, 20739, 57145, 1465, 1587, 2146, 2207, 32838, 336 or 52908 polypeptide” refers to a polypeptide having an amino acid sequence corresponding to an 9805, 2047, 46566, 57749, 577, 20739, 57145, 1465, 1587, 2146, 2207, 32838, 336 or 52908 molecule, whereas a “non-9805, 2047, 46566, 57749, 577, 20739, 57145, 1465, 1587, 2146, 2207, 32838, 336 or 52908 polypeptide” refers to a polypeptide having an amino acid sequence corresponding to a protein which is not substantially homologous to the 9805, 2047, 46566, 57749, 577, 20739, 57145, 1465, 1587, 2146, 2207, 32838, 336 or 52908 protein, e.g., a protein which is different from the 9805, 2047, 46566, 57749, 577, 20739, 57145, 1465, 1587, 2146, 2207, 32838, 336 or 52908 protein and which is derived from the same or a different organism. Within an 9805, 2047, 46566, 57749, 577, 20739, 57145, 1465, 1587, 2146, 2207, 32838, 336 or 52908 fusion protein the 9805, 2047, 46566, 57749, 577, 20739, 57145, 1465, 1587, 2146, 2207, 32838, 336 or 52908 polypeptide can correspond to all or a portion of an 9805, 2047, 46566, 57749, 577, 20739, 57145, 1465, 1587, 2146, 2207, 32838, 336 or 52908 protein. In a preferred embodiment, an 9805, 2047, 46566, 57749, 577, 20739, 57145, 1465, 1587, 2146, 2207, 32838, 336 or 52908 fusion protein comprises at least one biologically active portion of an 9805, 2047, 46566, 57749, 577, 20739, 57145, 1465, 1587, 2146, 2207, 32838, 336 or 52908 protein. In another preferred embodiment, an 9805, 2047, 46566, 57749, 577, 20739, 57145, 1465, 1587, 2146, 2207, 32838, 336 or 52908 fusion protein comprises at least two biologically active portions of an 9805, 2047, 46566, 57749, 577, 20739, 57145, 1465, 1587, 2146, 2207, 32838, 336 or 52908 protein. Within the fusion protein, the term “operatively linked” is intended to indicate that the 9805, 2047, 46566, 57749, 577, 20739, 57145, 1465, 1587, 2146, 2207, 32838, 336 or 52908 polypeptide and the non-9805, 2047, 46566, 57749, 577, 20739, 57145, 1465, 1587, 2146, 2207, 32838, 336 or 52908 polypeptide are fused in-frame to each other. The non-9805, 2047, 46566, 57749, 577, 20739, 57145, 1465, 1587, 2146, 2207, 32838, 336 or 52908 polypeptide can be fused to the N-terminus or C-terminus of the 9805, 2047, 46566, 57749, 577, 20739, 57145, 1465, 1587, 2146, 2207, 32838, 336 or 52908 polypeptide.

For example, in one embodiment, the fusion protein is a GST-9805, 2047, 46566, 57749, 577, 20739, 57145, 1465, 1587, 2146, 2207, 32838, 336 or 52908 fusion protein in which the 9805, 2047, 46566, 57749, 577, 20739, 57145, 1465, 1587, 2146, 2207, 32838, 336 or 52908 sequences are fused to the C-terminus of the GST sequences. Such fusion proteins can facilitate the purification of recombinant 9805, 2047, 46566, 57749, 577, 20739, 57145, 1465, 1587, 2146, 2207, 32838, 336 or 52908.

In another embodiment, this fusion protein is an 9805, 2047, 46566, 57749, 577, 20739, 57145, 1465, 1587, 2146, 2207, 32838, 336 or 52908 protein containing a heterologous signal sequence at its N-terminus. In certain host cells (e.g., mammalian host cells), expression and/or secretion of 9805, 2047, 46566, 57749, 577, 20739, 57145, 1465, 1587, 2146, 2207, 32838, 336 or 52908 can be increased through use of a heterologous signal sequence.

The 9805, 2047, 46566, 57749, 577, 20739, 57145, 1465, 1587, 2146, 2207, 32838, 336 or 52908 fusion proteins used in the methods of the invention can be incorporated into pharmaceutical compositions and administered to a subject in vivo. The 9805, 2047, 46566, 57749, 577, 20739, 57145, 1465, 1587, 2146, 2207, 32838, 336 or 52908 fusion proteins can be used to affect the bioavailability of an 9805, 2047, 46566, 57749, 577, 20739, 57145, 1465, 1587, 2146, 2207, 32838, 336 or 52908 substrate. Use of 9805, 2047, 46566, 57749, 577, 20739, 57145, 1465, 1587, 2146, 2207, 32838, 336 or 52908 fusion proteins may be useful therapeutically for the treatment of disorders caused by, for example, (i) aberrant modification or mutation of a gene encoding an 9805, 2047, 46566, 57749, 577, 20739, 57145, 1465, 1587, 2146, 2207, 32838, 336 or 52908 protein; (ii) mis-regulation of the 9805, 2047, 46566, 57749, 577, 20739, 57145, 1465, 1587, 2146, 2207, 32838, 336 or 52908 gene; and (iii) aberrant post-translational modification of an 9805, 2047, 46566, 57749, 577, 20739, 57145, 1465, 1587, 2146, 2207, 32838, 336 or 52908 protein.

Moreover, the 9805, 2047, 46566, 57749, 577, 20739, 57145, 1465, 1587, 2146, 2207, 32838, 336 or 52908-fusion proteins used in the methods of the invention can be used as immunogens to produce anti-9805, 2047, 46566, 57749, 577, 20739, 57145, 1465, 1587, 2146, 2207, 32838, 336 or 52908 antibodies in a subject, to purify 9805, 2047, 46566, 57749, 577, 20739, 57145, 1465, 1587, 2146, 2207, 32838, 336 or 52908 ligands and in screening assays to identify molecules which inhibit the interaction of 9805, 2047, 46566, 57749, 577, 20739, 57145, 1465, 1587, 2146, 2207, 32838, 336 or 52908 with an 9805, 2047, 46566, 57749, 577, 20739, 57145, 1465, 1587, 2146, 2207, 32838, 336 or 52908 substrate.

Preferably, an 9805, 2047, 46566, 57749, 577, 20739, 57145, 1465, 1587, 2146, 2207, 32838, 336 or 52908 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An 9805, 2047, 46566, 57749, 577, 20739, 57145, 1465, 1587, 2146, 2207, 32838, 336 or 52908-encoding nucleic acid can be cloned into such an expression vector such that the fusion moiety is linked in-frame to the 9805, 2047, 46566, 57749, 577, 20739, 57145, 1465, 1587, 2146, 2207, 32838, 336 or 52908 protein.

The present invention also pertains to the use of variants of the 9805, 2047, 46566, 57749, 577, 20739, 57145, 1465, 1587, 2146, 2207, 32838, 336 or 52908 proteins which function as either 9805, 2047, 46566, 57749, 577, 20739, 57145, 1465, 1587, 2146, 2207, 32838, 336 or 52908 agonists (mimetics) or as 9805, 2047, 46566, 57749, 577, 20739, 57145, 1465, 1587, 2146, 2207, 32838, 336 or 52908 antagonists. Variants of the 9805, 2047, 46566, 57749, 577, 20739, 57145, 1465, 1587, 2146, 2207, 32838, 336 or 52908 proteins can be generated by mutagenesis, e.g., discrete point mutation or truncation of an 9805, 2047, 46566, 57749, 577, 20739, 57145, 1465, 1587, 2146, 2207, 32838, 336 or 52908 protein. An agonist of the 9805, 2047, 46566, 57749, 577, 20739, 57145, 1465, 1587, 2146, 2207, 32838, 336 or 52908 proteins can retain substantially the same, or a subset, of the biological activities of the naturally occurring form of an 9805, 2047, 46566, 57749, 577, 20739, 57145, 1465, 1587, 2146, 2207, 32838, 336 or 52908 protein. An antagonist of an 9805, 2047, 46566, 57749, 577, 20739, 57145, 1465, 1587, 2146, 2207, 32838, 336 or 52908 protein can inhibit one or more of the activities of the naturally occurring form of the 9805, 2047, 46566, 57749, 577, 20739, 57145, 1465, 1587, 2146, 2207, 32838, 336 or 52908 protein by, for example, competitively modulating an 9805, 2047, 46566, 57749, 577, 20739, 57145, 1465, 1587, 2146, 2207, 32838, 336 or 52908-mediated activity of an 9805, 2047, 46566, 57749, 577, 20739, 57145, 1465, 1587, 2146, 2207, 32838, 336 or 52908 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 9805, 2047, 46566, 57749, 577, 20739, 57145, 1465, 1587, 2146, 2207, 32838, 336 or 52908 protein.

In one embodiment, variants of an 9805, 2047, 46566, 57749, 577, 20739, 57145, 1465, 1587, 2146, 2207, 32838, 336 or 52908 protein which function as either 9805, 2047, 46566, 57749, 577, 20739, 57145, 1465, 1587, 2146, 2207, 32838, 336 or 52908 agonists (mimetics) or as 9805, 2047, 46566, 57749, 577, 20739, 57145, 1465, 1587, 2146, 2207, 32838, 336 or 52908 antagonists can be identified by screening combinatorial libraries of mutants, e.g., truncation mutants, of an 9805, 2047, 46566, 57749, 577, 20739, 57145, 1465, 1587, 2146, 2207, 32838, 336 or 52908 protein for 9805, 2047, 46566, 57749, 577, 20739, 57145, 1465, 1587, 2146, 2207, 32838, 336 or 52908 protein agonist or antagonist activity. In one embodiment, a variegated library of 9805, 2047, 46566, 57749, 577, 20739, 57145, 1465, 1587, 2146, 2207, 32838, 336 or 52908 variants is generated by combinatorial mutagenesis at the nucleic acid level and is encoded by a variegated gene library. A variegated library of 9805, 2047, 46566, 57749, 577, 20739, 57145, 1465, 1587, 2146, 2207, 32838, 336 or 52908 variants can be produced by, for example, enzymatically ligating a mixture of synthetic oligonucleotides into gene sequences such that a degenerate set of potential 9805, 2047, 46566, 57749, 577, 20739, 57145, 1465, 1587, 2146, 2207, 32838, 336 or 52908 sequences is expressible as individual polypeptides, or alternatively, as a set of larger fusion proteins (e.g., for phage display) containing the set of 9805, 2047, 46566, 57749, 577, 20739, 57145, 1465, 1587, 2146, 2207, 32838, 336 or 52908 sequences therein. There are a variety of methods which can be used to produce libraries of potential 9805, 2047, 46566, 57749, 577, 20739, 57145, 1465, 1587, 2146, 2207, 32838, 336 or 52908 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 9805, 2047, 46566, 57749, 577, 20739, 57145, 1465, 1587, 2146, 2207, 32838, 336 or 52908 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of an 9805, 2047, 46566, 57749, 577, 20739, 57145, 1465, 1587, 2146, 2207, 32838, 336 or 52908 protein coding sequence can be used to generate a variegated population of 9805, 2047, 46566, 57749, 577, 20739, 57145, 1465, 1587, 2146, 2207, 32838, 336 or 52908 fragments for screening and subsequent selection of variants of an 9805, 2047, 46566, 57749, 577, 20739, 57145, 1465, 1587, 2146, 2207, 32838, 336 or 52908 protein. In one embodiment, a library of coding sequence fragments can be generated by treating a double stranded PCR fragment of an 9805, 2047, 46566, 57749, 577, 20739, 57145, 1465, 1587, 2146, 2207, 32838, 336 or 52908 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an. expression library can be derived which encodes N-terminal, C-terminal and internal fragments of various sizes of the 9805, 2047, 46566, 57749, 577, 20739, 57145, 1465, 1587, 2146, 2207, 32838, 336 or 52908 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 9805, 2047, 46566, 57749, 577, 20739, 57145, 1465, 1587, 2146, 2207, 32838, 336 or 52908 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 9805, 2047, 46566, 57749, 577, 20739, 57145, 1465, 1587, 2146, 2207, 32838, 336 or 52908 variants (Arkin and Youvan (1992) Proc. Natl. Acad. Sci. USA 89:7811-7815; Delagrave et al. (1993) Prot. Eng. 6(3):327-331).

The methods of the present invention further include the use of anti-9805, 2047, 46566, 57749, 577, 20739, 57145, 1465, 1587, 2146, 2207, 32838, 336 or 52908 antibodies. An isolated 9805, 2047, 46566, 57749, 577, 20739, 57145, 1465, 1587, 2146, 2207, 32838, 336 or 52908 protein, or a portion or fragment thereof, can be used as an immunogen to generate antibodies that bind 9805, 2047, 46566, 57749, 577, 20739, 57145, 1465, 1587, 2146, 2207, 32838, 336 or 52908 using standard techniques for polyclonal and monoclonal antibody preparation. A full-length 9805, 2047, 46566, 57749, 577, 20739, 57145, 1465, 1587, 2146, 2207, 32838, 336 or 52908 protein can be used or, alternatively, antigenic peptide fragments of 9805, 2047, 46566, 57749, 577, 20739, 57145, 1465, 1587, 2146, 2207, 32838, 336 or 52908 can be used as immunogens. The antigenic peptide of 9805, 2047, 46566, 57749, 577, 20739, 57145, 1465, 1587, 2146, 2207, 32838, 336 or 52908 comprises at least 8 amino acid residues of the amino acid sequence shown in SEQ ID NO:2, 4, 7, 10, 14, 17, 20, 23, 26, 29, 32, 35, 38 or 41 and encompasses an epitope of 9805, 2047, 46566, 57749, 577, 20739, 57145, 1465, 1587, 2146, 2207, 32838, 336 or 52908 such that an antibody raised against the peptide forms a specific immune complex with the 9805, 2047, 46566, 57749, 577, 20739, 57145, 1465, 1587, 2146, 2207, 32838, 336 or 52908 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 9805, 2047, 46566, 57749, 577, 20739, 57145, 1465, 1587, 2146, 2207, 32838, 336 or 52908 that are located on the surface of the protein, e.g., hydrophilic regions, as well as regions with high antigenicity.

An 9805, 2047, 46566, 57749, 577, 20739, 57145, 1465, 1587, 2146, 2207, 32838, 336 or 52908 immunogen is typically used to prepare antibodies by immunizing a suitable subject, (e.g., rabbit, goat, mouse, or other mammal) with the immunogen. An appropriate immunogenic preparation can contain, for example, recombinantly expressed 9805, 2047, 46566, 57749, 577, 20739, 57145, 1465, 1587, 2146, 2207, 32838, 336 or 52908 protein or a chemically synthesized 9805, 2047, 46566, 57749, 577, 20739, 57145, 1465, 1587, 2146, 2207, 32838, 336 or 52908 polypeptide. The preparation can further include an adjuvant, such as Freund's complete or incomplete adjuvant, or similar immunostimulatory agent. Immunization of a suitable subject with an immunogenic 9805, 2047, 46566, 57749, 577, 20739, 57145, 1465, 1587, 2146, 2207, 32838, 336 or 52908 preparation induces a polyclonal anti-9805, 2047, 46566, 57749, 577, 20739, 57145, 1465, 1587, 2146, 2207, 32838, 336 or 52908 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as an 9805, 2047, 46566, 57749, 577, 20739, 57145, 1465, 1587, 2146, 2207, 32838, 336 or 52908. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 9805, 2047, 46566, 57749, 577, 20739, 57145, 1465, 1587, 2146, 2207, 32838, 336 or 52908 molecules. The term “monoclonal antibody” or “monoclonal antibody composition”, as used herein, refers to a population of antibody molecules that contain only one species of an antigen binding site capable of immunoreacting with a particular epitope of 9805, 2047, 46566, 57749, 577, 20739, 57145, 1465, 1587, 2146, 2207, 32838, 336 or 52908. A monoclonal antibody composition thus typically displays a single binding affinity for a particular 9805, 2047, 46566, 57749, 577, 20739, 57145, 1465, 1587, 2146, 2207, 32838, 336 or 52908 protein with which it immunoreacts.

Polyclonal anti-9805, 2047, 46566, 57749, 577, 20739, 57145, 1465, 1587, 2146, 2207, 32838, 336 or 52908 antibodies can be prepared as described above by immunizing a suitable subject with an 9805, 2047, 46566, 57749, 577, 20739, 57145, 1465, 1587, 2146, 2207, 32838, 336 or 52908 immunogen. The anti-9805, 2047, 46566, 57749, 577, 20739, 57145, 1465, 1587, 2146, 2207, 32838, 336 or 52908 antibody titer in the immunized subject can be monitored over time by standard techniques, such as with an enzyme linked immunosorbent assay (ELISA) using immobilized 9805, 2047, 46566, 57749, 577, 20739, 57145, 1465, 1587, 2146, 2207, 32838, 336 or 52908. If desired, the antibody molecules directed against 9805, 2047, 46566, 57749, 577, 20739, 57145, 1465, 1587, 2146, 2207, 32838, 336 or 52908 can be isolated from the mammal (e.g., from the blood) and further purified by well known techniques, such as protein A chromatography to obtain the IgG fraction. At an appropriate time after immunization, e.g., when the anti-9805, 2047, 46566, 57749, 577, 20739, 57145, 1465, 1587, 2146, 2207, 32838, 336 or 52908 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lemer, E. A. (1981) Yale J. Biol. Med. 54:387-402; Gefter, M. L. et al. (1977) Somat. Cell Genet. 3:231-36). Briefly, an immortal cell line (typically a myeloma) is fused to lymphocytes (typically splenocytes) from a mammal immunized with an 9805, 2047, 46566, 57749, 577, 20739, 57145, 1465, 1587, 2146, 2207, 32838, 336 or 52908 immunogen as described above, and the culture supernatants of the resulting hybridoma cells are screened to identify a hybridoma producing a monoclonal antibody that binds 9805, 2047, 46566, 57749, 577, 20739, 57145, 1465, 1587, 2146, 2207, 32838, 336 or 52908.

Any of the many well known protocols used for fusing lymphocytes and immortalized cell lines can be applied for the purpose of generating an anti-9805, 2047, 46566, 57749, 577, 20739, 57145, 1465, 1587, 2146, 2207, 32838, 336 or 52908 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 9805, 2047, 46566, 57749, 577, 20739, 57145, 1465, 1587, 2146, 2207, 32838, 336 or 52908, e.g., using a standard ELISA assay.

Alternative to preparing monoclonal antibody-secreting hybridomas, a monoclonal anti-9805, 2047, 46566, 57749, 577, 20739, 57145, 1465, 1587, 2146, 2207, 32838, 336 or 52908 antibody can be identified and isolated by screening a recombinant combinatorial immunoglobulin library (e.g., an antibody phage display library) with 9805, 2047, 46566, 57749, 577, 20739, 57145, 1465, 1587, 2146, 2207, 32838, 336 or 52908 to thereby isolate immunoglobulin library members that bind 9805, 2047, 46566, 57749, 577, 20739, 57145, 1465, 1587, 2146, 2207, 32838, 336 or 52908. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-9805, 2047, 46566, 57749, 577, 20739, 57145, 1465, 1587, 2146, 2207, 32838, 336 or 52908 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-9805, 2047, 46566, 57749, 577, 20739, 57145, 1465, 1587, 2146, 2207, 32838, 336 or 52908 antibody can be used to detect 9805, 2047, 46566, 57749, 577, 20739, 57145, 1465, 1587, 2146, 2207, 32838, 336 or 52908 protein (e.g., in a cellular lysate or cell supernatant) in order to evaluate the abundance and pattern of expression of the 9805, 2047, 46566, 57749, 577, 20739, 57145, 1465, 1587, 2146, 2207, 32838, 336 or 52908 protein. Anti-9805, 2047, 46566, 57749, 577, 20739, 57145, 1465, 1587, 2146, 2207, 32838, 336 or 52908 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

IX. Electronic Apparatus Readable Media and Arrays

Electronic apparatus readable media comprising a 9805, 46566 or 57749 modulator of the present invention is also provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the 9805, 46566 or 57749 modulators of the present invention.

A variety of software programs and formats can be used to store the marker information of the present invention on the electronic apparatus readable medium. For example, the nucleic acid sequence corresponding to the 9805, 46566 or 57749 modulators can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the 9805, 46566 or 57749 modulators of the present invention.

By providing the 9805, 46566 or 57749 modulators of the invention in readable form, one can routinely access the marker sequence information for a variety of purposes. For example, one skilled in the art can use the nucleotide or amino acid sequences of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a pain disorder or a pre-disposition to a pain disroder, wherein the method comprises the steps of determining the presence or absence of a 9805, 46566 or 57749 modulator and based on the presence or absence of the 9805, 46566 or 57749 modulator, determining whether the subject has a pain disorder or a pre-disposition to a pain disorder and/or recommending a particular treatment for the pain disorder or pre-pain disorder condition.

The present invention further provides in an electronic system and/or in a network, a method for determining whether a subject has a pain disorder or a pre-disposition to a pain disorder associated with a 9805, 46566 or 57749 modulator wherein the method comprises the steps of determining the presence or absence of the 9805, 46566 or 57749 modulator, and based on the presence or absence of the 9805, 46566 or 57749 modulator, determining whether the subject has a pain disorder or a pre-disposition to a pain disorder, and/or recommending a particular treatment for the pain disorder or pre-pain disorder condition. The method may further comprise the step of receiving phenotypic information associated with the subject and/or acquiring from a network phenotypic information associated with the subject.

The present invention also provides in a network, a method for determining whether a subject has a pain disorder or a pre-disposition to a pain disorder associated with a 9805, 46566 or 57749 modulator, said method comprising the steps of receiving information associated with the 9805, 46566 or 57749 modulator receiving phenotypic information associated with the subject, acquiring information from the network corresponding to the 9805, 46566 or 57749 modulator and/or pain disorder, and based on one or more of the phenotypic information, the 9805, 46566 or 57749 modulator, and the acquired information, determining whether the subject has a pain disorder or a pre-disposition to a pain disorder. The method may further comprise the step of recommending a particular treatment for the pain disorder or pre-pain disorder condition.

The present invention also provides a business method for determining whether a subject has a pain disorder or a pre-disposition to a pain disorder, said method comprising the steps of receiving information associated with the 9805, 46566 or 57749 modulator, receiving phenotypic information associated with the subject, acquiring information from the network corresponding to the 9805, 46566 or 57749 modulator and/or pain disorder, and based on one or more of the phenotypic information, the 9805, 46566 or 57749 modulator, and the acquired information, determining whether the subject has a pain disorder or a pre-disposition to a pain disorder. The method may further comprise the step of recommending a particular treatment for the pain disorder or pre-pain disorder condition.

The invention also includes an array comprising a 9805, 46566 or 57749 modulator of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of pain disorder, progression of pain disorder, and processes, such a cellular transformation associated with pain disorder.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### 9805 Expression in Human Tissues

Materials and Methods

For analysis of human 9805 expression, the following methods were used.

Tissues were collected from various human tissues. Total RNA was prepared using the trizol method and treated with DNAse to remove contaminating genomic DNA. cDNA was synthesized using standard techniques. Mock cDNA synthesis in the absence of reverse transcriptase resulted in samples with no detectable PCR amplification of the control 18S RNA gene confirming efficient removal of genomic DNA contamination. 9805 expression was measured by TaqMan® quantitative PCR analysis, performed according to the manufacturer's directions (Perkin Elmer Applied Biosystems, Foster City, Calif.).

PCR probes were designed by PrimerExpress software (PE Biosystems) based on the sequence of human 9805 (SEQ ID NO:1).

To standardize the results between different tissues, two probes, distinguished by different fluorescent labels, were added to each sample. The differential labeling of the probe for the 9805 gene and the probe for 18S RNA as an internal control thus enabled their simultaneous measurement in the same well. Forward and reverse primers and the probes for both 18S RNA and human or murine 9805 were added to the TaqMan Universal PCR Master Mix (PE Applied Biosystems). Although the final concentration of primer and probe could vary, each was internally consistent within a given experiment. A typical experiment contained 200 nM of forward and reverse primers, plus 100 nM of the probe for the 18S RNA, and 4500 nM of each of the forward and reverse primers, plus 150 nM of the probe for murine 9805. TaqMan matrix experiments were carried out using an ABI PRISM 770 Sequence Detection System (PE Applied Biosystems). The thermal cycler conditions were as follows: hold for 2 minutes at 50° C. and 10 minutes at 95° C., followed by two-step PCR for 40 cycles of 95° C. for 15 seconds, followed by 60° C. for 1 minute.

The following method was used to quantitatively calculate human 9805 gene expression in the tissue samples, relative to the 18S RNA expression in the same tissue. The threshold values at which the PCR amplification started were determined using the manufacturer's software. PCR cycle number at threshold value was designated as CT. Relative expression was calculated as:
2−((CTtest−CT18S)tissue of interest−(CTtest−CT18S)lowest expressing tissue in panel)

Samples were run in duplicate and the averages of 2 relative expression determinations are shown. All probes were tested on serial dilutions of RNA from a tissue with high expression levels and only probes which gave relative expression levels that were linear to the amount of template cDNA with a slope similar to the slope for the internal control 18S were used.

Results

The expression of 9805 was examined in a variety of human tissues as described above. As indicated in FIG. 1, 9805 was most highly expressed in the hypothalamus and was also present in high levels in the brain, thymus, placenta, testes and spinal cord.

The results described above demonstrate that 9805 is a relatively nervous system specific gene.

### Example 2

### 9805 Expression in Tissues Derived from Animal Models for Pain

Materials and Methods

For analysis of rat 9805 expression, the methods described in Example 1 were used.

Results

TaqMan analysis using various rat tissues demonstrated that, 9805, like the human counterpart, is expressed in the same pattern of expression as in the human panel (described in Example 1).

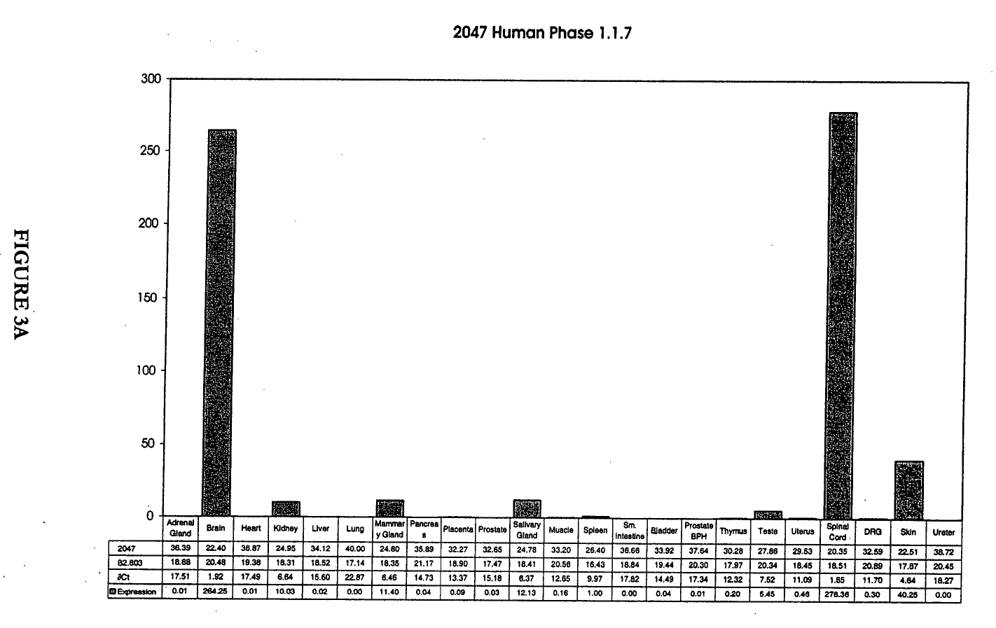

Expression of 9805 in animal models for pain/inflammation was also determined (FIG. 2). The results indicate that the 9805 gene is upregulated in dorsal root ganglia (DRG) at late time points after axotomy of the sciatic nerve. The 9805 gene is also upregulated in the dorsal root ganglia and the spinal cord of rats after chronic constrictive sciatic nerve injury (CCI) and in the dorsal root ganglia of monkeys after complete Freund's adjuvant (CFA)-induced inflammatory pain (FIG. 2).

### Example 3

### 2047 Expression in Human Tissues

Materials and Methods

For analysis of human 2047 expression, the methods described in Example 1 were used.

PCR probes were designed by PrimerExpress software (PE Biosystems) based on the sequence of human 2047 (SEQ ID NO:3).

Results

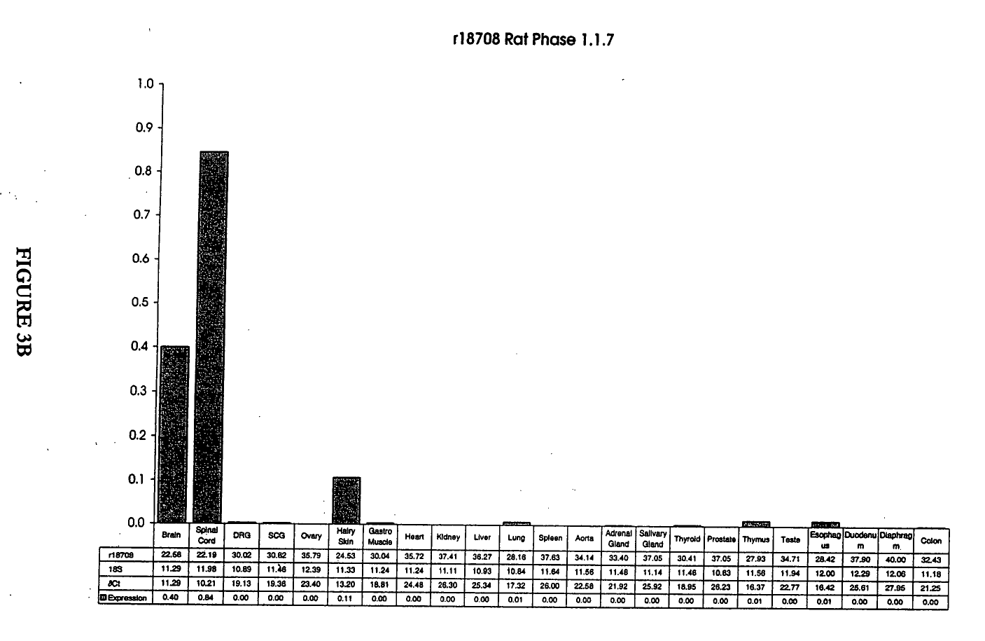

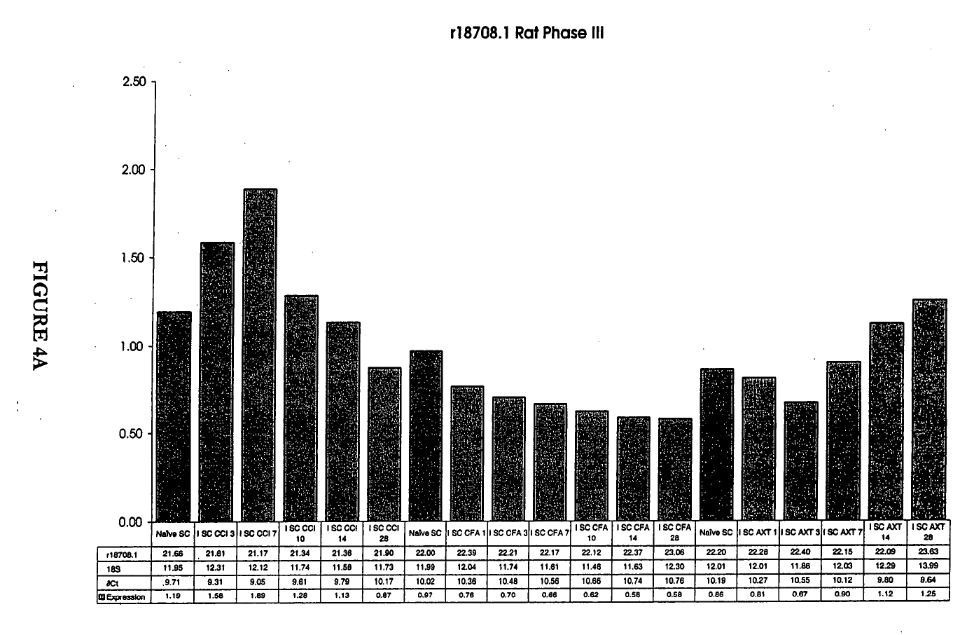

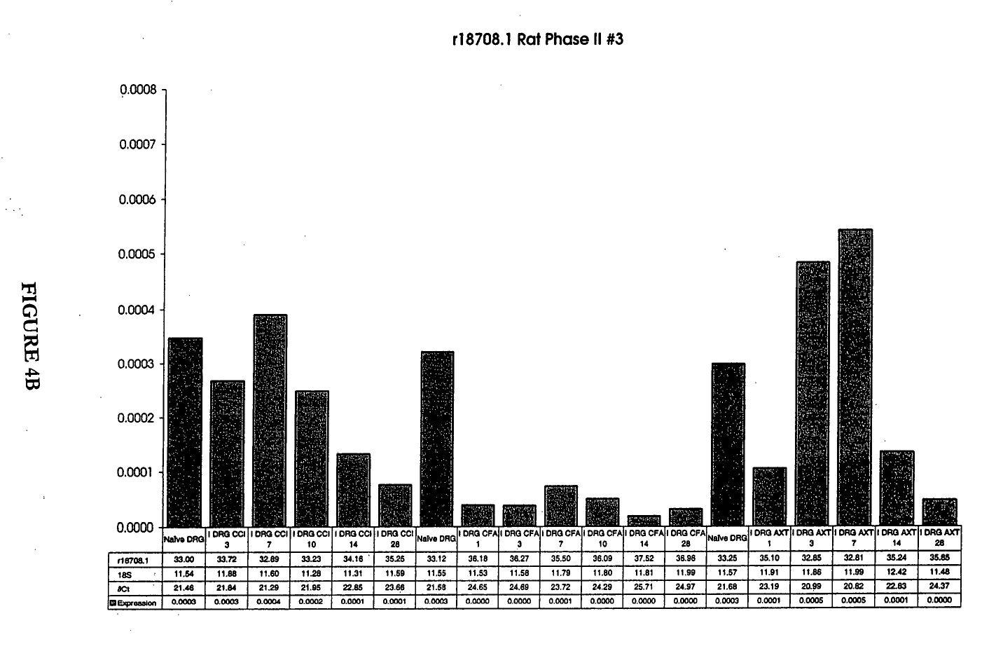

The expression of 2047 was examined in a variety of human tissues as described above. As indicated in FIG. 3A, human 2047 was most highly expressed in the spinal cord and the brain. In situ hybridization using a human probe confirmed the foregoing TaqMan data to verify expression of 2047 in the spinal cord and the brain. TaqMan analysis using various human tissues demonstrated that, 2047, like the rat counterpart, is expressed in the same pattern of expression as in the rat panel (described in Example 3) (see FIG. 3B).

### Example 4

### 2047 Expression in Tissues Derived from Animal Models for Pain

Materials and Methods

For analysis of rat 2047 expression, the methods described in Example 1 were used.

Results

Expression of 2047 in animal models for pain/inflammation was also determined (FIG. 4A-D).

One of the models used was the nerve ligation model (CCI), wherein chronic loose constriction of the sciatic nerve of the animal induces neuropathic pain. Nerve damage results in sensory hypersensitivity or prolonged lowering of the primary afferent nociceptor threshold (hyperalgesia). Furthermore, after axonal injury, mechanical and thermal allodynia develop. Another animal-based paradigm used was the complete Freund's adjuvant (CFA), induced inflammatory pain model. Injection of CFA in the rodent paw or the monkey knee joint induces an inflammatory response with the development of altered pain responses manifested as reduced threshold to noxious stimuli (hyperalgesia) and lowered thresholds to innocuous stimuli (allodynia).

The results of these experiments indicate that the 2047 gene is downregulated in dorsal root ganglia (DRG) after axotomy (see FIGS. 4B and 4C). The 2047 gene is also down regulated in the chronic constrictive sciatic nerve injury (CCI) model and in the complete Freund's adjuvant (CFA) induced injection model of inflammatory pain (see FIGS. 4A-D). The most dramatic changes were observed in the glabrous skin after CFA treatment where the 2047 gene was up-regulated 4- to 10-fold (see FIG. 4D). At late time points after CCI and axotomy, the 2047 gene was upregulated approximately 8-fold.

### Example 5

### 46566 Expression in Human Tissues

Materials and Methods

For analysis of human 46566 expression, the methods described in Example 1 were used.

PCR probes were designed by PrimerExpress software (PE Biosystems) based on the sequence of human 46566 (SEQ ID NO:6).

Results

46566 was most highly expressed in nervous tissues. The highest levels of 46566 expression were found in the brain, followed by the spinal cord and dorsal root ganglia (DRG). In situ hybridization using a human probe confirmed the foregoing TaqMan data.

The results described above demonstrate that 46566 is a relatively brain specific gene.

### Example 6

### 46566 Expression in Rat Tissues Derived from Animal Models for Pain

Materials and Methods

For analysis of rat 46566 expression, the methods described in Example 1 were used.

Results

TaqMan analysis using various rat tissues demonstrated that, 46566, like the human counterpart, is expressed in nervous tissues and showed the same pattern of expression as in the human panel (described in Example 1).

Expression of 46566 in animal models for pain/inflammation was also determined. One of the models used was the nerve ligation model (CCI), wherein chronic loose constriction of the sciatic nerve of the animal induces neuropathic pain. Nerve damage results in sensory hypersensitivity or prolonged lowering of the primary afferent nociceptor threshold (hyperalgesia). Furthermore, after axonal injury, mechanical and thermal allodynia develop. Another animal-based paradigm used was the complete Freund's adjuvant (CFA) model, wherein CFA injection in the rodent paw or monkey knee joint induces an inflammatory response with the development of altered pain responses manifested as reduced threshold to noxious stimuli (hyperalgesia) and lowered thresholds to innocuous stimuli (allodynia).

The results from these experiments indicate that there was no regulation of 46566 in the spinal cord of CCI animals and some down-regulation in the CFA induced model of inflammatory pain. A more pronounced down-regulation of 46566 was observed in animals after axotomy.

### Example 7

### 57749 Expression in Human Tissues

Materials and Methods

For analysis of human 57749 expression, the methods described in Example 1 were used.

PCR probes were designed by PrimerExpress software (PE Biosystems) based on the sequence of human 57749 (SEQ ID NO:9).

Results

The expression of 57749 was examined in a variety of human tissues as described above. 57749 was most highly expressed in the brain and was also present in high levels in the spinal cord and dorsal root ganglia.

The results described above demonstrate that 57749 is a relatively nervous system specific gene.

### Example 8

### 57749 Expression in Tissues Derived from Animal Models for Pain

Materials and Methods

For analysis of rat 57749 expression, the methods described in Example 1 were used.

Results

TaqMan analysis using various rat tissues demonstrated that, NCE-SLC24A, like the human counterpart, is expressed in the same pattern of expression as in the human panel (described in Example 1).

Expression of 57749 in animal models for pain/inflammation was also determined. The results indicate that the 57749 gene is upregulated in dorsal root ganglia (DRG) at late time points after axotomy of the sciatic nerve. The 57749 gene is also upregulated in the dorsal root ganglia and the spinal cord of rats after chronic constrictive sciatic nerve injury (CCI) and in the dorsal root ganglia of monkeys after complete Freund's adjuvant (CFA)-induced inflammatory pain.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a candidate compound capable of treating a pain disorder, comprising:
i) combining a compound to be tested with a host cell expressing a polypeptide selected from the group consisting of:
a) a polypeptide comprising the amino acid sequence of SEQ ID NO:2, 4, 7, 10, 14, 17, 20, 23, 26, 29, 32, 35, 38 or 41;
b) a polypeptide comprising an amino acid sequence which is at least 95% identical to the amino acid sequence of SEQ ID NO:2, 4, 7, 10, 14, 17, 20, 23, 26, 29, 32, 35, 38 or 41;
c) a polypeptide encoded by the nucleotide sequence set forth in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40; and
d) a polypeptide which is encoded by a nucleic acid molecule comprising a nucleotide sequence which is at least 95% identical to a nucleic acid comprising the nucleotide sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40;
under conditions suitable for binding of the test compound to the polypeptide; and
ii) detecting binding of the test compound to the polypeptide to thereby identify a compound which binds to the polypeptide;
thereby identifying a compound capable of treating a pain disorder.

2. The method of claim 1, wherein the compound is selected from the group consisting of a small molecule, a peptide or an antibody.

3. The method of claim 1, wherein the polypeptide further comprises heterologous sequences.

4. The method of claim 1, wherein the binding of the test compound to the polypeptide is detected by a method selected from the group consisting of:
a) a competition binding assay;
b) an immunoassay; and
c) a yeast two-hybrid assay.

5. The method of claim 1, wherein the binding of the test compound to the polypeptide is detected is by an assay for an activity of the polypeptide.

6. A method for identifying a candidate compound capable of modulating a pain signaling mechanism comprising:
i) combining a compound to be tested with a host cell expressing a polypeptide selected from the group consisting of:
a) a polypeptide comprising the amino acid sequence of SEQ ID NO:2, 4, 7, 10, 14, 17, 20, 23, 26, 29, 32, 35, 38 or 41;
b) a polypeptide comprising an amino acid sequence which is at least 95% identical to the amino acid sequence of SEQ ID NO:2, 4, 7, 10, 14, 17, 20, 23, 26, 29, 32, 35, 38 or 41;
c) a polypeptide encoded by the nucleotide sequence set forth in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40; and
d) a polypeptide which is encoded by a nucleic acid molecule comprising a nucleotide sequence which is at least 95% identical to a nucleic acid comprising the nucleotide sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40;
under conditions suitable for binding of the test compound to the polypeptide; and
ii) detecting binding of the test compound to the polypeptide to thereby identify a compound which binds to the polypeptide;
thereby identifying a compound capable of modulating a pain signaling mechanism.

7. The method of claim 6, wherein the compound is selected from the group consisting of a small molecule, a peptide or an antibody.

8. The method of claim 6, wherein the polypeptide further comprises heterologous sequences.

9. The method of claim 6, wherein the binding of the test compound to the polypeptide is detected by a method selected from the group consisting of:
a) a competition binding assay;
b) an immunoassay; and
c) a yeast two-hybrid assay.

10. The method of claim 6, wherein the binding of the test compound to the polypeptide is detected is by an assay for an activity of the polypeptide.

11. A method for identifying a compound capable of treating a pain disorder or capable of modulating a pain signaling mechanism, comprising:
i) combining a compound to be tested with a host cell expressing a polypeptide selected from the group consisting of:
a) a polypeptide comprising the amino acid sequence of SEQ ID NO:2, 4, 7, 10, 14, 17, 20, 23, 26, 29, 32, 35, 38 or 41;
b) a polypeptide comprising an amino acid sequence which is at least 95% identical to the amino acid sequence of SEQ ID NO:2, 4, 7, 10, 14, 17, 20, 23, 26, 29, 32, 35, 38 or 41;
c) a polypeptide encoded by the nucleotide sequence set forth in SEQ ID NO:1, 3, 5, 6, 8, 9, 11, 12, 13, 15, 16, 18, 19, 21, 22, 24, 25, 27, 28, 30, 31, 33, 34, 36, 37, 39 or 40; and
d) a polypeptide which is encoded by a nucleic acid molecule comprising a nucleotide sequence which is at least 95% identical to a nucleic acid comprising the nucleotide sequence of SEQ ID NO:1, 3, 5, 6, 8, 9, 11, 12, 13, 15, 16, 18, 19, 21, 22, 24, 25, 27, 28, 30, 31, 33, 34, 36, 37, 39 or 40; and
ii) assaying the ability of the test compound to modulate the expression of a 9805, 2047, 46566, 57749, 577, 20739, 57145, 1465, 1587, 2146, 2207, 32838, 336 and 52908 nucleic acid or the activity of a 9805, 2047, 46566, 57749, 577, 20739, 57145, 1465, 1587, 2146, 2207, 32838, 336 and 52908 polypeptide, thereby identifying a compound capable of treating a pain disorder or capable of modulating a pain signaling mechanism.

12. The method of claim 11, wherein the cell is a brain cell, neuron, or cell derived from spinal cord or dorsal root ganglion.

13. The method of claim 11, wherein the compound is a small organic molecule, peptide, antibody or antisense nucleic acid molecule.

14. A method for treating a subject having a pain disorder characterized by aberrant 9805, 2047, 46566, 57749, 577, 20739, 57145, 1465, 1587, 2146, 2207, 32838, 336 and 52908 polypeptide activity or aberrant 9805, 2047, 46566, 57749, 577, 20739, 57145, 1465, 1587, 2146, 2207, 32838, 336 and 52908 nucleic acid expression comprising administering to the subject a 9805, 2047, 46566, 57749, 577, 20739, 57145, 1465, 1587, 2146, 2207, 32838, 336 and 52908 modulator, thereby treating said subject having a pain disorder.

15. The method of claim 14, wherein said pain disorder includes inflammatory pain, chronic pain, neuropathic pain, causalgia, fibromyalgia, cancer pain, migraine/headache pain and tissue pain.

16. The method of claim 14, wherein said 9805, 2047, 46566, 57749, 577, 20739, 57145, 1465, 1587, 2146, 2207, 32838, 336 and 52908 modulator is administered in a pharmaceutically acceptable formulation.
